SEQUENCE LISTING (113 sequences)

SEQ ID NO: 1 - RNA, 76 nt, Artificial Sequence
  sgRNA1.0 non-targeting region
guuuuagagcuagaaauagcaaguuaaaauaaggcuaguccguuaucaacuugaaaaaguggcaccgagucggugc

SEQ ID NO: 2 - RNA, 86 nt, Artificial Sequence
  sgRNA2.0 non-targeting region
guuuaagagcuaugcuggaaacagcauagcaaguuuaaauaaggcuaguccguuaucaacuugaaaaaguggcaccgagucggugc

SEQ ID NO: 3 - RNA, 96 nt, Artificial Sequence
  sgRNA2.1 non-targeting region
guuuaagagcuaugcuggaaacagcauagcaaguuuaaauaaggcuaguccguuaucaacuuugcuggaaacagcaaaguggcaccgagucggugc

SEQ ID NO: 4 - RNA, 42 nt, Artificial Sequence
  wt SpCas9 crRNA
  misc_feature (1)..(20): n is a, c, g, or u
nnnnnnnnnnnnnnnnnnnnguuuuagagcuaugcuguuuug

SEQ ID NO: 5 - RNA, 86 nt, Artificial Sequence
  wt tracrRNA
ggaaccauucaaaacagcauagcaaguuaaaauaaggcuaguccguuaucaacuugaaaaaguggcaccgagucggugcuuuuuuu

SEQ ID NO: 6 - RNA, 102 nt, Artificial Sequence
  sgRNA1
  misc_feature (1)..(20): n is a, c, g, or u
nnnnnnnnnnnnnnnnnnnnguuuuagagcuagaaauagcaaguuaaaauaaggcuaguccguuaucaacuugaaaaaguggcaccgagucggugcuuuuuu

SEQ ID NO: 7 - RNA, 112 nt, Artificial Sequence
  sgRNA2.0
  misc_feature (1)..(20): n is a, c, g, or u
nnnnnnnnnnnnnnnnnnnnguuuaagagcuaugcuggaaacagcauagcaaguuuaaauaaggcuaguccguuaucaacuugaaaaaguggcaccgagucggugcuuuuuu

SEQ ID NO: 8 - RNA, 122 nt, Artificial Sequence
  sgRNA2.1
  misc_feature (1)..(20): n is a, c, g, or u
nnnnnnnnnnnnnnnnnnnnguuuaagagcuaugcuggaaacagcauagcaaguuuaaauaaggcuaguccguuaucaacuuugcuggaaacagcaaaguggcaccgagucggugcuuuuuu

SEQ ID NO: 9 - RNA, 96 nt, Artificial Sequence
  sgRNA1 Aff1
ccuucagcucagugacagugguuuuagagcuagaaauagcaaguuaaaauaaggcuaguccguuaucaacuugaaaaaguggcaccgagucggugc

SEQ ID NO: 10 - RNA, 106 nt, Artificial Sequence
  sgRNA2.0 Aff1
ccuucagcucagugacagugguuuaagagcuaugcuggaaacagcauagcaaguuuaaauaaggcuaguccguuaucaacuugaaaaaguggcaccgagucggugc

SEQ ID NO: 11 - RNA, 116 nt, Artificial Sequence
  sgRNA2.1 Aff1
ccuucagcucagugacagugguuuaagagcuaugcuggaaacagcauagcaaguuuaaauaaggcuaguccguuaucaacuuugcuggaaacagcaaaguggcaccgagucggugc

SEQ ID NO: 12 - RNA, 96 nt, Artificial Sequence
  sgRNA1 pcsk9
agcagcagcggcggcaacagguuuuagagcuagaaauagcaaguuaaaauaaggcuaguccguuaucaacuugaaaaaguggcaccgagucggugc

SEQ ID NO: 13 - RNA, 106 nt, Artificial Sequence
  sgRNA2.0 pcsk9
agcagcagcggcggcaacagguuuaagagcuaugcuggaaacagcauagcaaguuuaaauaaggcuaguccguuaucaacuugaaaaaguggcaccgagucggugc

SEQ ID NO: 14 - RNA, 116 nt, Artificial Sequence
  sgRNA2.1 pcsk9
agcagcagcggcggcaacagguuuaagagcuaugcuggaaacagcauagcaaguuuaaauaaggcuaguccguuaucaacuuugcuggaaacagcaaaguggcaccgagucggugc

SEQ ID NO: 15 - RNA, 96 nt, Artificial Sequence
  sgRNA1 serping1
guuugcaagacagaggcgaaguuuuagagcuagaaauagcaaguuaaaauaaggcuaguccguuaucaacuugaaaaaguggcaccgagucggugc

SEQ ID NO: 16 - RNA, 106 nt, Artificial Sequence
  sgRNA2.0 serping1
guuugcaagacagaggcgaaguuuaagagcuaugcuggaaacagcauagcaaguuuaaauaaggcuaguccguuaucaacuugaaaaaguggcaccgagucggugc

SEQ ID NO: 17 - RNA, 116 nt, Artificial Sequence
  sgRNA2.1 serping1
guuugcaagacagaggcgaaguuuaagagcuaugcuggaaacagcauagcaaguuuaaauaaggcuaguccguuaucaacuuugcuggaaacagcaaaguggcaccgagucggugc

SEQ ID NO: 18 - RNA, 96 nt, Artificial Sequence
  sgRNA1 vegfa(site 3)
ggugagugagugugugcgugguuuuagagcuagaaauagcaaguuaaaauaaggcuaguccguuaucaacuugaaaaaguggcaccgagucggugc

SEQ ID NO: 19 - RNA, 106 nt, Artificial Sequence
  sgRNA2.0 vegfa(site 3)
ggugagugagugugugcgugguuuaagagcuaugcuggaaacagcauagcaaguuuaaauaaggcuaguccguuaucaacuugaaaaaguggcaccgagucggugc

SEQ ID NO: 20 - RNA, 116 nt, Artificial Sequence
  sgRNA2.1 vegfa(site 3)
ggugagugagugugugcgugguuuaagagcuaugcuggaaacagcauagcaaguuuaaauaaggcuaguccguuaucaacuuugcuggaaacagcaaaguggcaccgagucggugc

SEQ ID NO: 21 - RNA, 96 nt, Artificial Sequence
  sgRNA1 eGFP
cugcuucauguuagggguagguuuuagagcuagaaauagcaaguuaaaauaaggcuaguccguuaucaacuugaaaaaguggcaccgagucggugc

SEQ ID NO: 22 - RNA, 106 nt, Artificial Sequence
  sgRNA2.0 eGFP
cugcuucauguuagggguagguuuaagagcuaugcuggaaacagcauagcaaguuuaaauaaggcuaguccguuaucaacuugaaaaaguggcaccgagucggugc

SEQ ID NO: 23 - RNA, 116 nt, Artificial Sequence
  sgRNA2.1 eGFP
cugcuucauguuagggguagguuuaagagcuaugcuggaaacagcauagcaaguuuaaauaaggcuaguccguuaucaacuuugcuggaaacagcaaaguggcaccgagucggugc

SEQ ID NO: 24 - DNA, 42 bp, Artificial Sequence
  MVA9
gagtgctggccgaggccatgagcgactataaggaccacgacg

SEQ ID NO: 25 - DNA, 48 bp, Artificial Sequence
  MVA10
cctgcggccgctccggaattccatgtgtcactttttcttttttgcctg

SEQ ID NO: 26 - DNA, 41 bp, Artificial Sequence
  MVA64
gatccgctagcgctaccggtaccaccatggactataaggac

SEQ ID NO: 27 - DNA, 71 bp, Artificial Sequence
  MVA65
ctcgagatctgagtccggtctgcacaatcggatagttctggctaaagttctttttcttttttgcctggccg

SEQ ID NO: 28 - DNA, 38 bp, Artificial Sequence
  MVA66
tcactttttttcaggttggaccggtcgccaccatggtg

SEQ ID NO: 29 - DNA, 41 bp, Artificial Sequence
  MVA67
gctgatcagcgagctctaggcttgtacagctcgtccatgcc

SEQ ID NO: 30 - DNA, 54 bp, Artificial Sequence
  MVA111
aattatgttttaaaatggactatcatatgcttaccgtaacttgaaagtatttcg

SEQ ID NO: 31 - DNA, 32 bp, Artificial Sequence
  MVA113
ggggcgtacttggcatatgatacacttgatgt

SEQ ID NO: 32 - DNA, 49 bp, Artificial Sequence
  MVA114
cttatttaaacttgctatgctgtttccagcatagctcttaaacaggtct

SEQ ID NO: 33 - DNA, 39 bp, Artificial Sequence
  MVA115
ggaaacagcatagcaagtttaaataaggctagtccgtta

SEQ ID NO: 34 - DNA, 49 bp, Artificial Sequence
  MVA141
ccactttgctgtttccagcaaagttgataacggactagccttatttaaa

SEQ ID NO: 35 - DNA, 36 bp, Artificial Sequence
  MVA142
aactttgctggaaacagcaaagtggcaccgagtcgg

SEQ ID NO: 36 - DNA, 20 bp, Artificial Sequence
  AFF1 - Sequence
ccttcagctcagtgacagtg

SEQ ID NO: 37 - DNA, 20 bp, Artificial Sequence
  SERPING1 - Sequence
gtttgcaagacagaggcgaa

SEQ ID NO: 38 - DNA, 20 bp, Artificial Sequence
  eGFPmut - Sequence
ctgcttcatgttaggggtag

SEQ ID NO: 39 - DNA, 20 bp, Artificial Sequence
  VEGFA site 3 - Sequence
ggtgagtgagtgtgtgcgtg

SEQ ID NO: 40 - DNA, 20 bp, Artificial Sequence
  Pcsk9 - Sequence
agcagcagcggcggcaacag

SEQ ID NO: 41 - DNA, 21 bp, Artificial Sequence
  AFF1 - Fw primer
agaggcttgcacttaactggt

SEQ ID NO: 42 - DNA, 21 bp, Artificial Sequence
  eGFPmut - Fw primer
attgacgcaaatgggcggtag

SEQ ID NO: 43 - DNA, 23 bp, Artificial Sequence
  VEGFA site 3 - Fw primer
ctcagctccacaaacttggtgcc

SEQ ID NO: 44 - DNA, 23 bp, Artificial Sequence
  Pcsk9 - Fw primer
tctctctttctgaggctagagga

SEQ ID NO: 45 - DNA, 20 bp, Artificial Sequence
  AFF1 - Rv primer
aaacactgggctcgtcagtt

SEQ ID NO: 46 - DNA, 25 bp, Artificial Sequence
  eGFPmut - Rv primer
ttacttgtacagctcgtccatgccg

SEQ ID NO: 47 - DNA, 18 bp, Artificial Sequence
  VEGFA site 3 - Rv primer
agcccgccgcaatgaagg

SEQ ID NO: 48 - DNA, 22 bp, Artificial Sequence
  Pcsk9 - Rv primer
ccccaagtctatttccaagccg

SEQ ID NO: 49 - DNA, 7751 bp, Artificial Sequence
  pCCL/PGK-eGFP
aagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgacgatggtcgagtaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctctagcgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggcctcgagggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgc

SEQ ID NO: 50 - DNA, 11007 bp, Artificial Sequence
  pGFP-PH-gagpol-D64V
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacggtaccgcgggcccgggatccatggactcgggccgggacttcctgaccctgcacggcctacaggatgatgaggatctacaggcgctgctgaagggcagccagctcctgaaggtgaagtccagctcatggaggagagagcgcttctacaagttgcaggaggactgcaagaccatctggcaggagtcccgcaaggtcatgcggaccccggagtcccagctgttctccatcgaggacattcaggaggtgcgaatggggcaccgcacggagggtctggagaagttcgcccgtgatgtgcccgaggaccgctgcttctccattgtcttcaaggaccagcgcaatacactagacctcatcgccccatcgccagctgatgcccagcactgggtgctggggctgcacaagatcatccaccactcaggctccatggaccagcgtcagaagctacagcactggattcactcctgcttgcgaaaagctgacaaaaacaaggacaacaagatgagcttcaaggagctgcagaacttcctgaaggagctcaacatccagcctcgagccgaattcaccctggccgccagggccagcgtgctgagcggcggcgagctggacaggtgggagaagatcaggctgaggcccggcggcaagaagaagtataagctgaagcacatcgtgtgggccagcagggagctggagaggttcgccgtgaaccccggcctgctggagaccagcgagggctgcaggcagatcctgggccagctgcagcccagcctgcagaccggcagcgaggagctgaggagcctgtacaacaccgtggccaccctgtactgcgtgcaccagaggatcgagatcaaggacaccaaggaggccctggacaagatcgaggaggagcagaacaagtccaagaagaaggcccagcaggccgccgccgacaccggccacagcagccaggtgagccagaactaccccatcgtgcagaacatccagggccagatggtgcaccaggccatcagccccaggaccctgaacgcctgggtgaaggtggtggaggagaaggccttcagccccgaggtgatccccatgttcagcgccctgagcgagggagccaccccccaggacctgaacaccatgctgaacaccgtgggcggccaccaggccgccatgcagatgctgaaggagaccatcaacgaggaggccgccgagtgggacagggtgcaccccgtgcacgccggccccatcgcccccggccagatgagggagccccgcggcagcgacatcgccggcaccaccagcaccctgcaggagcagatcggctggatgaccaacaacccccccatccccgtgggcgaaatctacaagaggtggatcatcctgggcctgaacaagatcgtgaggatgtacagccccaccagcatcctggatatcaggcagggccccaaagagcccttcagggactacgtggacaggttctacaagaccctgcgcgccgagcaggccagccaggaggtgaagaactggatgaccgagaccctgctggtgcagaacgccaaccccgactgcaagaccatcctgaaggccctgggacccgccgccaccctggaggagatgatgaccgcctgccagggcgtgggcggccccggccacaaggccagggtgctggccgaggccatgagccaggtgaccaacaccgccaccatcatgatgcagaggggcaacttcaggaaccagaggaagatggtgaagtgcttcaactgcggcaaggagggccacaccgccaggaactgccgcgcccccaggaagaagggctgctggaagtgcggcaaggagggccaccagatgaaggactgcaccgagaggcaggctaattttttagggaagatctggccttcctacaagggaaggccagggaattttcttcagagcagaccagagccaacagccccaccatttcttcagagcagaccagagccaacagccccaccagaagagagcttcaggtctggggtagagacaacaactccccctcagaagcaggagccgatagacaaggaactgtatcctttaacttccctcagatcactctttggcaacgacccctcgtcacaataaagatcggtggccagctgaaggaggccctgctggacaccggcgccgacgacaccgtgctggaggagatgagcctgcccggcaggtggaagcccaagatgatcggcggcatcggcggcttcatcaaggtgaggcagtacgaccagatcctgatcgagatctgcggccacaaggccatcggcaccgtgctggtgggacctacacctgtgaacatcatcggcaggaacctgctgacccagatcggctgcaccctgaacttccccatcagccccatcgagaccgtgcccgtgaagctgaagcccggcatggacggccctaaggtgaagcagtggcccctgaccgaggagaagatcaaggccctggtggagatctgcaccgagatggagaaggagggcaagatcagcaagatcggccccgagaacccctacaacacccccgtgttcgccatcaagaagaaggacagcaccaagtggaggaagctggtggacttcagggagctgaacaagaggacccaggacttctgggaggtgcagctgggcatcccccaccccgccggcctgaagaagaagaagagcgtgaccgtgctggacgtgggcgacgcctacttcagcgtgcccctggacgaggacttcaggaagtataccgccttcaccatccccagcatcaacaacgagacccccggcatccgctaccagtacaacgtgctgccccagggctggaagggcagccccgccatcttccagagcagcatgacaaagatcctggagcccttcaagaagcagaaccccgacatcgtgatctatcagtacatggacgacctgtacgtgggcagcgacctggagatcggccagcacaggaccaagatcgaggagctgaggcagcacctgctgaggtggggcctgaccacccccgacaagaagcaccagaaggagcccccattcctgtggatgggctacgagctgcaccccgacaagtggaccgtgcagcccatcgtgctgcccgagaaggacagctggaccgtgaacgacattcagaagctggtgggcaagctgaactgggccagccagatctaccccggcatcaaggtgaggcagctgtgcaagctgctgaggggcacaaaggctctgaccgaggtgatccccctgaccgaggaggccgagctggagctggccgagaacagggagatcctgaaggagcccgtgcacggcgtgtactacgaccccagcaaggacctgatcgccgagatccagaagcagggccagggccagtggacctaccagatctaccaggagcccttcaagaacctgaagaccggcaagtacgcccgcatgcgcggcgcccacaccaacgacgtgaagcagctgaccgaggccgtgcagaagatcaccaccgagagcatcgtgatctggggcaagactcctaagttcaagctgcccatccagaaggagacctgggagacctggtggaccgagtactggcaggccacctggattcccgagtgggagttcgtgaacacccctcccctggtgaagctgtggtatcagctggagaaggagcccatcgtgggcgccgagaccttctacgtggacggcgccgccaacagggagaccaagctgggcaaggccggctacgtgaccaacaagggccgccagaaggtggtgcccctgaccaacaccaccaaccagaagaccgagctgcaggctatctacctggccctgcaggactcaggcctggaggtgaacatcgtgaccgacagccagtacgccctgggcatcatccaggcccagcccgacaagagcgagagcgagctggtgaaccagatcatcgagcagctgatcaagaaggagaaggtgtacctggcctgggtgcccgcccacaagggcatcggcggcaacgagcaggtggacaagctggtgagcgccggcatcaggaagatcctgttcctggacggcatcgacaaggcccaggacgagcacgagaagtaccacagcaactggagggctatggctagcgacttcaacctgcctcccgtggtggctaaggagatcgtggccagctgcgacaagtgccagctgaagggcgaggccatgcacggccaggtggactgcagccccggcatctggcagctggtttgcacccacctggagggcaaggtgatcctggtggccgtgcacgtggcctccggctacatcgaggccgaggtgatccccgccgagaccggccaggagaccgcctacttcctgctgaagctggccggccgctggcccgtgaagaccatccacaccgacaacggcagcaacttcaccagcgccaccgtgaaggccgcctgctggtgggccggcatcaagcaggagttcggcatcccctacaacccccagtctcagggcgtggtggagagcatgaacaaggagctgaagaagatcatcggccaggtgagggaccaggccgagcacctgaagaccgccgtgcagatggccgtgttcatccacaacttcaagaggaagggcggcatcggcggctacagcgccggcgagaggatcgtggacatcatcgccaccgacatccagaccaaggagctgcagaagcagatcaccaagatccagaacttcagggtgtactacagggacagcaggaaccctctgtggaagggccccgccaagctgctgtggaagggcgagggcgccgtggtgatccaggacaacagcgacatcaaggtggtgcccaggaggaaggccaagatcatcagggactacggcaagcagatggccggcgacgactgcgtggcctccaggcaggacgaggactgactcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcgcgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 51 - DNA, 10183 bp, Artificial Sequence
  pLentiGuide-puro
taacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccagagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagacggttgtaaatgagcacacaaaatacacatgctaaaatattatattctatgacctttataaaatcaaccaaaatcttctttttaataactttagtatcaataattagaatttttatgttcctttttgcaaacttttaataaaaatgagcaaaataaaaaaacgctagttttagtaactcgcgttgttttcttcacctttaataatagctactccaccacttgttcctaagcggtcagctcctgcttcaatcattttttgagcatcttcaaatgttctaactccaccagctgctttaactaaagcattgtctttaacaactgacttcattagtttaacatcttcaaatgttgcacctgattttgaaaatcctgttgatgttttaacaaattctaatccagcttcaacagctatttcacaagctttcatgatttcttcttttgttaataaacaattttccataatacatttaacaacatgtgatccagctgctttttttacagctttcatgtcttctaaaactaattcataatttttgtcttttaatgcaccaatatttaataccatatcaatttctgttgcaccatctttaattgcttcagaaacttcgaatgcttttgtagctgttgtgcatgcacctagaggaaaacctacaacatttgttattcctacatttgtgccttttaataattctttacaatagcttgttcaatatgaattaacacaaactgttgcaaaatcaaattcaattgcttcatcacataattgtttaatttcagctttcgtagcatcttgttttaataatgtgtgatctatatatttgtttagtttcattttttctcctatatattcatttttaattttaattctttaataatttcgtctactttaactttagcgttttgaacagattcaccaacacctataaaataaatttttagtttaggttcagttccacttgggcgaacagcaaatcatgacttatcttctaaataaaattttagtaagtcttgtcctggcatattatacattccatcgatgtagtcttcaacattaacaactttaagtccagcaatttgagttaagggtgttgctctcaatgatttcattaatggttcaatttttaatttcttttcttctggtttaaaattcaagtttaaagtgaaagtgtaatatgcacccatttctttaaataaatcttctaaatagtctactaatgttttattttgttttttataaaatcaagcagcctctgctattaatatagaagcttgtattccatctttatctctagctgagtcatcaattacatatccataactttcttcataagcaaaaacaaaatttaatccgttatcttcttctttagcaatttctctacccattcatttaaatccagttaaagtttttacaatattaactccatatttttcatgagcgattctatcacccaaatcacttgttacaaaacttgaatatagagccggattttttggaatgctatttaagcgttttagatttgataattttcaatcaattaaaattggtcctgtttgatttccatctaatcttacaaaatgaccatcatgttttattgccattccaaatctgtcagcatctgggtcattcataataataatatctgcatcatgtttaataccatattcaagcggtatttttcatgcaggatcaaattctggatttggatttacaacatttttaaatgtttcatcttcaaatgcatgctcttcaacctcaataacgttatatcctgattcacgtaatatttttggggtaaatttagttcctgttccattaactgcgctaaaaataatttttaaatcttttttagcttcttgctcttttttgtacgtctctgttttagagctagaaatagcaagttaaaataaggctagtccgttatcaacttgaaaaagtggcaccgagtcggtgcttttttaagcttggcgtaactagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggcgccggctcgagggggcccgggtgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgccgtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaaca

SEQ ID NO: 52 - DNA, 13191 bp, Artificial Sequence
  pMDLg/p-RRE-D64V-PCS-SpCas9
ggatcccctgagggggcccccatgggctagaggatccggcctcggcctctgcataaataaaaaaaattagtcagccatgagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgaattcgagatctgccgccgccatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaggacagtatgatcagatactcatagaaatctgtggacataaagctataggtacagtattagtaggacctacacctgtcaacataattggaagaaatctgttgactcagattggttgcactttaaattttcccattagccctattgagactgtaccagtaaaattaaagccaggaatggatggcccaaaagttaaacaatggccattgacagaagaaaaaataaaagcattagtagaaatttgtacagagatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactgcccatacaaaaggaaacatgggaaacatggtggacagagtattggcaagccacctggattcctgagtgggagtttgttaatacccctcccttagtgaaattatggtaccagttagagaaagaacccatagtaggagcagaaaccttctatgtagatggggcagctaacagggagactaaattaggaaaagcaggatatgttactaatagaggaagacaaaaagttgtcaccctaactgacacaacaaatcagaagactgagttacaagcaatttatctagctttgcaggattcgggattagaagtaaacatagtaacagactcacaatatgcattaggaatcattcaagcacaaccagatcaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaagtactatttttagatggaatagataaggcccaagatgaacatgagaaatatcacagtaattggagagcaatggctagtgattttaacctgccacctgtagtagcaaaagaaatagtagccagctgtgataaatgtcagctaaaaggagaagccatgcatggacaagtagactgtagtccaggaatatggcaactagtttgtacacatttagaaggaaaagttatcctggtagcagttcatgtagccagtggatatatagaagcagaagttattccagcagaaacagggcaggaaacagcatattttcttttaaaattagcaggaagatggccagtaaaaacaatacatactgacaatggcagcaatttcaccggtgctacggttagggccgcctgttggtgggcagggatcaagcaggaatttggcattccctacaatccccaaagccaaggagtagtagaatctatgaataatgaattaaagaaaattataggacaggtaagagatcaggctgaacaccttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggataaggccagagtgctggccgaggccatgagcgactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtgacacatggaattccggagcggccgcaggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttccgcggaattcaccccaccagtgcaggctgcctatcagaaagtggtggctggtgtggctaatgccctggcccacaagtttcactaagctcgcttccttgctgtccaatttctattaaaggttccttggttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagccctgatgcctatgccttattcatccctcagaaaaggattcaagtagaggcttgatttggaggttaaagtttggctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgt

SEQ ID NO: 53 - DNA, 10707 bp, Artificial Sequence
  pMDLg/p-PCS-hyPBase
ggatcccctgagggggcccccatgggctagaggatccggcctcggcctctgcataaataaaaaaaattagtcagccatgagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgaattcgagatctgccgccgccatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaggacagtatgatcagatactcatagaaatctgtggacataaagctataggtacagtattagtaggacctacacctgtcaacataattggaagaaatctgttgactcagattggttgcactttaaattttcccattagccctattgagactgtaccagtaaaattaaagccaggaatggatggcccaaaagttaaacaatggccattgacagaagaaaaaataaaagcattagtagaaatttgtacagagatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactgcccatacaaaaggaaacatgggaaacatggtggacagagtattggcaagccacctggattcctgagtgggagtttgttaatacccctcccttagtgaaattatggtaccagttagagaaagaacccatagtaggagcagaaaccttctatgtagatggggcagctaacagggagactaaattaggaaaagcaggatatgttactaatagaggaagacaaaaagttgtcaccctaactgacacaacaaatcagaagactgagttacaagcaatttatctagctttgcaggattcgggattagaagtaaacatagtaacagactcacaatatgcattaggaatcattcaagcacaaccagatcaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaagtactatttttagatggaatagataaggcccaagatgaacatgagaaatatcacagtaattggagagcaatggctagtgattttaacctgccacctgtagtagcaaaagaaatagtagccagctgtgataaatgtcagctaaaaggagaagccatgcatggacaagtagactgtagtccaggaatatggcaactagtttgtacacatttagaaggaaaagttatcctggtagcagttcatgtagccagtggatatatagaagcagaagttattccagcagagacagggcaggaaacagcatattttctcttaaaattagcaggaagatggccagtaaaaacaatacatacagacaatggcagcaatttcaccagtactacggttaaggccgcctgttggtgggcagggatcaagcaggaatttggcattccctacaatccccaaagccaaggagtagtagaatctatgaataatgaattaaagaaaattataggacaggtaagagatcaggctgaacaccttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggataaggccagagtgctggccgaggccatgagcatgggcagcagcctggacgacgagcacatcctgagcgccctgctgcagagcgacgacgagctggtcggcgaggacagcgacagcgaggtgagcgaccacgtgagcgaggacgacgtgcagtccgacaccgaggaggccttcatcgacgaggtgcacgaggtgcagcctaccagcagcggctccgagatcctggacgagcagaacgtgatcgagcagcccggcagctccctggccagcaacaggatcctgaccctgccccagaggaccatcaggggcaagaacaagcactgctggtccacctccaagcccaccaggcggagcagggtgtccgccctgaacatcgtgagaagccagaggggccccaccaggatgtgcaggaacatctacgaccccctgctgtgcttcaagctgttcttcaccgacgagatcatcagcgagatcgtgaagtggaccaacgccgagatcagcctgaagaggcgggagagcatgacctccgccaccttcagggacaccaacgaggacgagatctacgccttcttcggcatcctggtgatgaccgccgtgaggaaggacaaccacatgagcaccgacgacctgttcgacagatccctgagcatggtgtacgtgagcgtgatgagcagggacagattcgacttcctgatcagatgcctgaggatggacgacaagagcatcaggcccaccctgcgggagaacgacgtgttcacccccgtgagaaagatctgggacctgttcatccaccagtgcatccagaactacacccctggcgcccacctgaccatcgacgagcagctgctgggcttcaggggcaggtgccccttcagggtctatatccccaacaagcccagcaagtacggcatcaagatcctgatgatgtgcgacagcggcaccaagtacatgatcaacggcatgccctacctgggcaggggcacccagaccaacggcgtgcccctgggcgagtactacgtgaaggagctgtccaagcccgtccacggcagctgcagaaacatcacctgcgacaactggttcaccagcatccccctggccaagaacctgctgcaggagccctacaagctgaccatcgtgggcaccgtgagaagcaacaagagagagatccccgaggtcctgaagaacagcaggtccaggcccgtgggcaccagcatgttctgcttcgacggccccctgaccctggtgtcctacaagcccaagcccgccaagatggtgtacctgctgtccagctgcgacgaggacgccagcatcaacgagagcaccggcaagccccagatggtgatgtactacaaccagaccaagggcggcgtggacaccctggaccagatgtgcagcgtgatgacctgcagcagaaagaccaacaggtggcccatggccctgctgtacggcatgatcaacatcgcctgcatcaacagcttcatcatctacagccacaacgtgagcagcaagggcgagaaggtgcagagccggaaaaagttcatgcggaacctgtacatgggcctgacctccagcttcatgaggaagaggctggaggcccccaccctgaagagatacctgagggacaacatcagcaacatcctgcccaaagaggtgcccggcaccagcgacgacagcaccgaggagcccgtgatgaagaagaggacctactgcacctactgtcccagcaagatcagaagaaaggccagcgccagctgcaagaagtgtaagaaggtcatctgccgggagcacaacatcgacatgtgccagagctgtttctgacacatggaattccggagcggccgcaggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttccgcggaattcaccccaccagtgcaggctgcctatcagaaagtggtggctggtgtggctaatgccctggcccacaagtttcactaagctcgcttccttgctgtccaatttctattaaaggttccttggttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagccctgatgcctatgccttattcatccctcagaaaaggattcaagtagaggcttgatttggaggttaaagtttggctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgt

SEQ ID NO: 54 - DNA, 14588 bp, Artificial Sequence
  pSpCas9-PH-gagpol-D64V
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtaccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagaactttagccagaactatccgattgtgcagaccggactcagatctcgagctcaagcttcgaattctgcagtcgacggtaccgcgggcccgggatccatggactcgggccgggacttcctgaccctgcacggcctacaggatgatgaggatctacaggcgctgctgaagggcagccagctcctgaaggtgaagtccagctcatggaggagagagcgcttctacaagttgcaggaggactgcaagaccatctggcaggagtcccgcaaggtcatgcggaccccggagtcccagctgttctccatcgaggacattcaggaggtgcgaatggggcaccgcacggagggtctggagaagttcgcccgtgatgtgcccgaggaccgctgcttctccattgtcttcaaggaccagcgcaatacactagacctcatcgccccatcgccagctgatgcccagcactgggtgctggggctgcacaagatcatccaccactcaggctccatggaccagcgtcagaagctacagcactggattcactcctgcttgcgaaaagctgacaaaaacaaggacaacaagatgagcttcaaggagctgcagaacttcctgaaggagctcaacatccagcctcgagccgaattcaccctggccgccagggccagcgtgctgagcggcggcgagctggacaggtgggagaagatcaggctgaggcccggcggcaagaagaagtataagctgaagcacatcgtgtgggccagcagggagctggagaggttcgccgtgaaccccggcctgctggagaccagcgagggctgcaggcagatcctgggccagctgcagcccagcctgcagaccggcagcgaggagctgaggagcctgtacaacaccgtggccaccctgtactgcgtgcaccagaggatcgagatcaaggacaccaaggaggccctggacaagatcgaggaggagcagaacaagtccaagaagaaggcccagcaggccgccgccgacaccggccacagcagccaggtgagccagaactaccccatcgtgcagaacatccagggccagatggtgcaccaggccatcagccccaggaccctgaacgcctgggtgaaggtggtggaggagaaggccttcagccccgaggtgatccccatgttcagcgccctgagcgagggagccaccccccaggacctgaacaccatgctgaacaccgtgggcggccaccaggccgccatgcagatgctgaaggagaccatcaacgaggaggccgccgagtgggacagggtgcaccccgtgcacgccggccccatcgcccccggccagatgagggagccccgcggcagcgacatcgccggcaccaccagcaccctgcaggagcagatcggctggatgaccaacaacccccccatccccgtgggcgaaatctacaagaggtggatcatcctgggcctgaacaagatcgtgaggatgtacagccccaccagcatcctggatatcaggcagggccccaaagagcccttcagggactacgtggacaggttctacaagaccctgcgcgccgagcaggccagccaggaggtgaagaactggatgaccgagaccctgctggtgcagaacgccaaccccgactgcaagaccatcctgaaggccctgggacccgccgccaccctggaggagatgatgaccgcctgccagggcgtgggcggccccggccacaaggccagggtgctggccgaggccatgagccaggtgaccaacaccgccaccatcatgatgcagaggggcaacttcaggaaccagaggaagatggtgaagtgcttcaactgcggcaaggagggccacaccgccaggaactgccgcgcccccaggaagaagggctgctggaagtgcggcaaggagggccaccagatgaaggactgcaccgagaggcaggctaattttttagggaagatctggccttcctacaagggaaggccagggaattttcttcagagcagaccagagccaacagccccaccatttcttcagagcagaccagagccaacagccccaccagaagagagcttcaggtctggggtagagacaacaactccccctcagaagcaggagccgatagacaaggaactgtatcctttaacttccctcagatcactctttggcaacgacccctcgtcacaataaagatcggtggccagctgaaggaggccctgctggacaccggcgccgacgacaccgtgctggaggagatgagcctgcccggcaggtggaagcccaagatgatcggcggcatcggcggcttcatcaaggtgaggcagtacgaccagatcctgatcgagatctgcggccacaaggccatcggcaccgtgctggtgggacctacacctgtgaacatcatcggcaggaacctgctgacccagatcggctgcaccctgaacttccccatcagccccatcgagaccgtgcccgtgaagctgaagcccggcatggacggccctaaggtgaagcagtggcccctgaccgaggagaagatcaaggccctggtggagatctgcaccgagatggagaaggagggcaagatcagcaagatcggccccgagaacccctacaacacccccgtgttcgccatcaagaagaaggacagcaccaagtggaggaagctggtggacttcagggagctgaacaagaggacccaggacttctgggaggtgcagctgggcatcccccaccccgccggcctgaagaagaagaagagcgtgaccgtgctggacgtgggcgacgcctacttcagcgtgcccctggacgaggacttcaggaagtataccgccttcaccatccccagcatcaacaacgagacccccggcatccgctaccagtacaacgtgctgccccagggctggaagggcagccccgccatcttccagagcagcatgacaaagatcctggagcccttcaagaagcagaaccccgacatcgtgatctatcagtacatggacgacctgtacgtgggcagcgacctggagatcggccagcacaggaccaagatcgaggagctgaggcagcacctgctgaggtggggcctgaccacccccgacaagaagcaccagaaggagcccccattcctgtggatgggctacgagctgcaccccgacaagtggaccgtgcagcccatcgtgctgcccgagaaggacagctggaccgtgaacgacattcagaagctggtgggcaagctgaactgggccagccagatctaccccggcatcaaggtgaggcagctgtgcaagctgctgaggggcacaaaggctctgaccgaggtgatccccctgaccgaggaggccgagctggagctggccgagaacagggagatcctgaaggagcccgtgcacggcgtgtactacgaccccagcaaggacctgatcgccgagatccagaagcagggccagggccagtggacctaccagatctaccaggagcccttcaagaacctgaagaccggcaagtacgcccgcatgcgcggcgcccacaccaacgacgtgaagcagctgaccgaggccgtgcagaagatcaccaccgagagcatcgtgatctggggcaagactcctaagttcaagctgcccatccagaaggagacctgggagacctggtggaccgagtactggcaggccacctggattcccgagtgggagttcgtgaacacccctcccctggtgaagctgtggtatcagctggagaaggagcccatcgtgggcgccgagaccttctacgtggacggcgccgccaacagggagaccaagctgggcaaggccggctacgtgaccaacaagggccgccagaaggtggtgcccctgaccaacaccaccaaccagaagaccgagctgcaggctatctacctggccctgcaggactcaggcctggaggtgaacatcgtgaccgacagccagtacgccctgggcatcatccaggcccagcccgacaagagcgagagcgagctggtgaaccagatcatcgagcagctgatcaagaaggagaaggtgtacctggcctgggtgcccgcccacaagggcatcggcggcaacgagcaggtggacaagctggtgagcgccggcatcaggaagatcctgttcctggacggcatcgacaaggcccaggacgagcacgagaagtaccacagcaactggagggctatggctagcgacttcaacctgcctcccgtggtggctaaggagatcgtggccagctgcgacaagtgccagctgaagggcgaggccatgcacggccaggtggactgcagccccggcatctggcagctggtttgcacccacctggagggcaaggtgatcctggtggccgtgcacgtggcctccggctacatcgaggccgaggtgatccccgccgagaccggccaggagaccgcctacttcctgctgaagctggccggccgctggcccgtgaagaccatccacaccgacaacggcagcaacttcaccagcgccaccgtgaaggccgcctgctggtgggccggcatcaagcaggagttcggcatcccctacaacccccagtctcagggcgtggtggagagcatgaacaaggagctgaagaagatcatcggccaggtgagggaccaggccgagcacctgaagaccgccgtgcagatggccgtgttcatccacaacttcaagaggaagggcggcatcggcggctacagcgccggcgagaggatcgtggacatcatcgccaccgacatccagaccaaggagctgcagaagcagatcaccaagatccagaacttcagggtgtactacagggacagcaggaaccctctgtggaagggccccgccaagctgctgtggaagggcgagggcgccgtggtgatccaggacaacagcgacatcaaggtggtgcccaggaggaaggccaagatcatcagggactacggcaagcagatggccggcgacgactgcgtggcctccaggcaggacgaggactgactcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcgcgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 55 - DNA, 11232 bp, Artificial Sequence
  pZFNL(GFP)-PH-gagpol-D64V
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccgggcgccaccatggcccccaagaagaagaggaaggtgggaatccatggggtaccgggcaagaagaagcagcacatctgccacatccagggctgtggtaaagtttacggccgctccgacgacctgacccgccacctgcgctggcacaccggcgagaggcctttcatgtgtacatggtcctactgtggtaaacgcttcacccagtccggcgccctggcccgccacaagcgtacccacaccggtgagaagaaatttgcttgtccggaatgtccgaagcgcttcatgcgctccgaccacctgtcccgccacatcaagacccaccagaacaagaagggtggatcccaactagtcaaaagtgaactggaggagaagaaatctgaacttcgtcataaattgaaatatgtgcctcatgaatatattgaattaattgaaattgccagaaattccactcaggatagaattcttgaaatgaaggtaatggaattttttatgaaagtttatggatatagaggtaaacatttgggtggatcaaggaaaccggacggagcaatttatactgtcggatctcctattgattacggtgtgatcgtggatactaaagcttatagcggaggttataatctgccaattggccaagcagatgaaatgcaacgatatgtcgaagaaaatcaaacacgaaacaaacatatcaaccctaatgaatggtggaaagtctatccatcttctgtaacggaatttaagtttttatttgtgagtggtcactttaaaggaaactacaaagctcagcttacacgattaaatcatatcactaattgtaatggagctgttcttagtgtagaagagcttttaattggtggagaaatgattaaagccggcacattaaccttagaggaagtgagacggaaatttaataacggcgagataaactttagccagaactatccgattgtgcagcccggactcagatctcgagctcaagcttcgaattctgcagtcgacggtaccgcgggcccgggatccatggactcgggccgggacttcctgaccctgcacggcctacaggatgatgaggatctacaggcgctgctgaagggcagccagctcctgaaggtgaagtccagctcatggaggagagagcgcttctacaagttgcaggaggactgcaagaccatctggcaggagtcccgcaaggtcatgcggaccccggagtcccagctgttctccatcgaggacattcaggaggtgcgaatggggcaccgcacggagggtctggagaagttcgcccgtgatgtgcccgaggaccgctgcttctccattgtcttcaaggaccagcgcaatacactagacctcatcgccccatcgccagctgatgcccagcactgggtgctggggctgcacaagatcatccaccactcaggctccatggaccagcgtcagaagctacagcactggattcactcctgcttgcgaaaagctgacaaaaacaaggacaacaagatgagcttcaaggagctgcagaacttcctgaaggagctcaacatccagcctcgagccgaattcaccctggccgccagggccagcgtgctgagcggcggcgagctggacaggtgggagaagatcaggctgaggcccggcggcaagaagaagtataagctgaagcacatcgtgtgggccagcagggagctggagaggttcgccgtgaaccccggcctgctggagaccagcgagggctgcaggcagatcctgggccagctgcagcccagcctgcagaccggcagcgaggagctgaggagcctgtacaacaccgtggccaccctgtactgcgtgcaccagaggatcgagatcaaggacaccaaggaggccctggacaagatcgaggaggagcagaacaagtccaagaagaaggcccagcaggccgccgccgacaccggccacagcagccaggtgagccagaactaccccatcgtgcagaacatccagggccagatggtgcaccaggccatcagccccaggaccctgaacgcctgggtgaaggtggtggaggagaaggccttcagccccgaggtgatccccatgttcagcgccctgagcgagggagccaccccccaggacctgaacaccatgctgaacaccgtgggcggccaccaggccgccatgcagatgctgaaggagaccatcaacgaggaggccgccgagtgggacagggtgcaccccgtgcacgccggccccatcgcccccggccagatgagggagccccgcggcagcgacatcgccggcaccaccagcaccctgcaggagcagatcggctggatgaccaacaacccccccatccccgtgggcgaaatctacaagaggtggatcatcctgggcctgaacaagatcgtgaggatgtacagccccaccagcatcctggatatcaggcagggccccaaagagcccttcagggactacgtggacaggttctacaagaccctgcgcgccgagcaggccagccaggaggtgaagaactggatgaccgagaccctgctggtgcagaacgccaaccccgactgcaagaccatcctgaaggccctgggacccgccgccaccctggaggagatgatgaccgcctgccagggcgtgggcggccccggccacaaggccagggtgctggccgaggccatgagccaggtgaccaacaccgccaccatcatgatgcagaggggcaacttcaggaaccagaggaagatggtgaagtgcttcaactgcggcaaggagggccacaccgccaggaactgccgcgcccccaggaagaagggctgctggaagtgcggcaaggagggccaccagatgaaggactgcaccgagaggcaggctaattttttagggaagatctggccttcctacaagggaaggccagggaattttcttcagagcagaccagagccaacagccccaccatttcttcagagcagaccagagccaacagccccaccagaagagagcttcaggtctggggtagagacaacaactccccctcagaagcaggagccgatagacaaggaactgtatcctttaacttccctcagatcactctttggcaacgacccctcgtcacaataaagatcggtggccagctgaaggaggccctgctggacaccggcgccgacgacaccgtgctggaggagatgagcctgcccggcaggtggaagcccaagatgatcggcggcatcggcggcttcatcaaggtgaggcagtacgaccagatcctgatcgagatctgcggccacaaggccatcggcaccgtgctggtgggacctacacctgtgaacatcatcggcaggaacctgctgacccagatcggctgcaccctgaacttccccatcagccccatcgagaccgtgcccgtgaagctgaagcccggcatggacggccctaaggtgaagcagtggcccctgaccgaggagaagatcaaggccctggtggagatctgcaccgagatggagaaggagggcaagatcagcaagatcggccccgagaacccctacaacacccccgtgttcgccatcaagaagaaggacagcaccaagtggaggaagctggtggacttcagggagctgaacaagaggacccaggacttctgggaggtgcagctgggcatcccccaccccgccggcctgaagaagaagaagagcgtgaccgtgctggacgtgggcgacgcctacttcagcgtgcccctggacgaggacttcaggaagtataccgccttcaccatccccagcatcaacaacgagacccccggcatccgctaccagtacaacgtgctgccccagggctggaagggcagccccgccatcttccagagcagcatgacaaagatcctggagcccttcaagaagcagaaccccgacatcgtgatctatcagtacatggacgacctgtacgtgggcagcgacctggagatcggccagcacaggaccaagatcgaggagctgaggcagcacctgctgaggtggggcctgaccacccccgacaagaagcaccagaaggagcccccattcctgtggatgggctacgagctgcaccccgacaagtggaccgtgcagcccatcgtgctgcccgagaaggacagctggaccgtgaacgacattcagaagctggtgggcaagctgaactgggccagccagatctaccccggcatcaaggtgaggcagctgtgcaagctgctgaggggcacaaaggctctgaccgaggtgatccccctgaccgaggaggccgagctggagctggccgagaacagggagatcctgaaggagcccgtgcacggcgtgtactacgaccccagcaaggacctgatcgccgagatccagaagcagggccagggccagtggacctaccagatctaccaggagcccttcaagaacctgaagaccggcaagtacgcccgcatgcgcggcgcccacaccaacgacgtgaagcagctgaccgaggccgtgcagaagatcaccaccgagagcatcgtgatctggggcaagactcctaagttcaagctgcccatccagaaggagacctgggagacctggtggaccgagtactggcaggccacctggattcccgagtgggagttcgtgaacacccctcccctggtgaagctgtggtatcagctggagaaggagcccatcgtgggcgccgagaccttctacgtggacggcgccgccaacagggagaccaagctgggcaaggccggctacgtgaccaacaagggccgccagaaggtggtgcccctgaccaacaccaccaaccagaagaccgagctgcaggctatctacctggccctgcaggactcaggcctggaggtgaacatcgtgaccgacagccagtacgccctgggcatcatccaggcccagcccgacaagagcgagagcgagctggtgaaccagatcatcgagcagctgatcaagaaggagaaggtgtacctggcctgggtgcccgcccacaagggcatcggcggcaacgagcaggtggacaagctggtgagcgccggcatcaggaagatcctgttcctggacggcatcgacaaggcccaggacgagcacgagaagtaccacagcaactggagggctatggctagcgacttcaacctgcctcccgtggtggctaaggagatcgtggccagctgcgacaagtgccagctgaagggcgaggccatgcacggccaggtggactgcagccccggcatctggcagctggtttgcacccacctggagggcaaggtgatcctggtggccgtgcacgtggcctccggctacatcgaggccgaggtgatccccgccgagaccggccaggagaccgcctacttcctgctgaagctggccggccgctggcccgtgaagaccatccacaccgacaacggcagcaacttcaccagcgccaccgtgaaggccgcctgctggtgggccggcatcaagcaggagttcggcatcccctacaacccccagtctcagggcgtggtggagagcatgaacaaggagctgaagaagatcatcggccaggtgagggaccaggccgagcacctgaagaccgccgtgcagatggccgtgttcatccacaacttcaagaggaagggcggcatcggcggctacagcgccggcgagaggatcgtggacatcatcgccaccgacatccagaccaaggagctgcagaagcagatcaccaagatccagaacttcagggtgtactacagggacagcaggaaccctctgtggaagggccccgccaagctgctgtggaagggcgagggcgccgtggtgatccaggacaacagcgacatcaaggtggtgcccaggaggaaggccaagatcatcagggactacggcaagcagatggccggcgacgactgcgtggcctccaggcaggacgaggactgactcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcgcgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 56 - DNA, 4947 bp, Artificial Sequence
  pU6-Chimeric_BB-CBh-eGFP
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 57 - DNA, 4957 bp, Artificial Sequence
  pU6-SpCas9_sgRNA2.0-CBh-eGFP
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgtttaagagctatgctggaaacagcatagcaagtttaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 58 - DNA, 4967 bp, Artificial Sequence
  pU6-SpCas9_sgRNA2.1-CBh-eGFP
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgtttaagagctatgctggaaacagcatagcaagtttaaataaggctagtccgttatcaactttgctggaaacagcaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 59 - DNA, 21 bp, Artificial Sequence
  Forward primer
ccttcagctcagtgacagtgg

SEQ ID NO: 60 - DNA, 18 bp, Artificial Sequence
  Reverse primer
ccgactcggtgccacttt

SEQ ID NO: 61 - DNA, 26 bp, Artificial Sequence
  Probe
aaataaggctagtccgttatcaactt

SEQ ID NO: 62 - DNA, 5 bp, Artificial Sequence
  Extension A
ugcug

SEQ ID NO: 63 - DNA, 5 bp, Artificial Sequence
  Extension B
cagca

SEQ ID NO: 64 - DNA, 30 bp, Artificial Sequence
  SERPING1 - Fw primer
ctcatctgccgcactgtcagaaattactct

SEQ ID NO: 65 - DNA, 30 bp, Artificial Sequence
  SERPING1 - Rv primer
gattggtgactcttatgggagtgtccaaca

SEQ ID NO: 66 - RNA, 21 nt, Artificial Sequence
  P1
ccuucagcucagugacagugg

SEQ ID NO: 67 - RNA, 26 nt, Artificial Sequence
  P2
aaauaaggcuaguccguuaucaacuu

SEQ ID NO: 68 - RNA, 18 nt, Artificial Sequence
  P3
aaaguggcaccgagucgg

SEQ ID NO: 69 - DNA, 20 bp, Artificial Sequence
  ddPCR_Pcsk9 Fwd primer
cagcagcggcggcaacaggt

SEQ ID NO: 70 - DNA, 20 bp, Artificial Sequence
  ddPCR_VEGFA Fwd primer
cctggaagatgtccaccagt

SEQ ID NO: 71 - DNA, 20 bp, Artificial Sequence
  ddPCR Serping1 Fwd primer
ttgcaagacagaggcgaagt

SEQ ID NO: 72 - DNA, 20 bp, Artificial Sequence
  ChIP_Pcsk9 Fwd primer
ggctcccgttctctctctct

SEQ ID NO: 73 - DNA, 19 bp, Artificial Sequence
  ChIP_Pcsk9 Rev primer
ctcgggaaggacatggacg

SEQ ID NO: 74 - DNA, 22 bp, Artificial Sequence
  ChIP Reference Fwd primer
ctgctcctgactgggtaatgaa

SEQ ID NO: 75 - DNA, 22 bp, Artificial Sequence
  ChIP Reference Rev primer
attttgtgctgcataacctcct

SEQ ID NO: 76 - DNA, 21 bp, Artificial Sequence
  Seq_Vegfa_Nested1_Fwd primer
gagcccagggagcaaaggtca

SEQ ID NO: 77 - DNA, 24 bp, Artificial Sequence
  Seq_Vegfa_Nested1_Rev primer
ggttcctggttgtgcttagtggga

SEQ ID NO: 78 - DNA, 25 bp, Artificial Sequence
  Seq_Vegfa_Nested2-P1_Fwd primer
aaaggtcacgaaagcagatggtcaa

SEQ ID NO: 79 - DNA, 24 bp, Artificial Sequence
  Seq_Vegfa_Nested2-P1_Rev primer
gtgtatatacatagctgtccccgg

SEQ ID NO: 80 - DNA, 22 bp, Artificial Sequence
  Seq_Vegfa_Nested2-P2_Fwd primer
agagcttcggcagggaagtaca

SEQ ID NO: 81 - DNA, 24 bp, Artificial Sequence
  Seq_Vegfa_Nested2-P2_Rev primer
tatttgatgagtggctgttggcct

SEQ ID NO: 82 - DNA, 20 bp, Artificial Sequence
  Seq_Vegfa_Nested2-P3_Fwd primer
ttgtaccactgtcctcctgc

SEQ ID NO: 83 - DNA, 20 bp, Artificial Sequence
  Seq_Vegfa_Nested2-P3_Rev primer
gtggagctgtaaggagtggt

SEQ ID NO: 84 - DNA, 38 bp, Artificial Sequence
  SA1
tgtcgacgatatcttcgaacatggtgagcaagggcgag

SEQ ID NO: 85 - DNA, 43 bp, Artificial Sequence
  SA2
acggccgctatgctttactgctacacattgatcctagcagaag

SEQ ID NO: 86 - DNA, 58 bp, Artificial Sequence
  JH9
ctcttcctcatctccgggcctttcgacctctagcgaatgtcctttattccagtggccg

SEQ ID NO: 87 - DNA, 42 bp, Artificial Sequence
  JH10
gctgggccaccaggcattactgctccagtcgttcatgaggac

SEQ ID NO: 88 - DNA, 39 bp, Artificial Sequence
  JH11
cctcatgaacgactggagcagtaatgcctggtggcccag

SEQ ID NO: 89 - DNA, 47 bp, Artificial Sequence
  JH12
ccattgctggatgtctcgtgctatgaaaagacagcaagcttaggtct

SEQ ID NO: 90 - DNA, 41 bp, Artificial Sequence
  JH13
agcttgctgtcttttcatagcacgagacatccagcaatggg

SEQ ID NO: 91 - DNA, 44 bp, Artificial Sequence
  JH14
gagaagtttgttgcgccggatccggctggtgaaagggcaggcag

SEQ ID NO: 92 - DNA, 39 bp, Artificial Sequence
  JH15
tgcctgccctttcaccagccggatccggcgcaacaaact

SEQ ID NO: 93 - DNA, 49 bp, Artificial Sequence
  JH16
gcggaatttacggccgctatgctttactgccttgtacagctcgtccatg

SEQ ID NO: 94 - DNA, 20 bp, Artificial Sequence
  D2eGFP - sequence
cggtgaacagctcctcgccc

SEQ ID NO: 95 - DNA, 20 bp, Artificial Sequence
  PCSK9 off target - Fw primer
gtacaaggctccacaggtca

SEQ ID NO: 96 - DNA, 21 bp, Artificial Sequence
  PCSK9 off target - Rv primer
ctcctgctctaggagaggtgt

SEQ ID NO: 97 - DNA, 20 bp, Artificial Sequence
  VEGFA site 1 off target1 - Fw primer
ggagactgtttggcctctgt

SEQ ID NO: 98 - DNA, 20 bp, Artificial Sequence
  VEGFA  site 1 off target1 - Rv primer
aatagtcccctgttcgtgcc

SEQ ID NO: 99 - DNA, 20 bp, Artificial Sequence
  VEGFA  site 1 off target2 - Fw primer
gctggtccctgtggattaca

SEQ ID NO: 100 - DNA, 20 bp, Artificial Sequence
  VEGFA  site 1 off target2 - Rv primer
tgagagacttcggggacaga

SEQ ID NO: 101 - DNA, 20 bp, Artificial Sequence
  Fah - sequence
ggatggtcctcatgaacgac

SEQ ID NO: 102 - DNA, 23 bp, Artificial Sequence
  Fah - Fw primer
gttgccaaattggctccacttac

SEQ ID NO: 103 - DNA, 20 bp, Artificial Sequence
  Fah - Rv primer
ccacacccacagagtcagaa

SEQ ID NO: 104 - RNA, 116 nt, Artificial Sequence
  sgRNA2.1 d2eGFP
gggcgaggagcuguucaccgguuuaagagcuaugcuggaaacagcauagcaaguuuaaauaaggcuaguccguuaucaacuuugcuggaaacagcaaaguggcaccgagucggugc

SEQ ID NO: 105 - RNA, 116 nt, Artificial Sequence
  sgRNA2.1 Fah
ggaugguccucaugaacgacguuuaagagcuaugcuggaaacagcauagcaaguuuaaauaaggcuaguccguuaucaacuuugcuggaaacagcaaaguggcaccgagucggugc

SEQ ID NO: 106 - DNA, 8251 bp, Artificial Sequence
  pCCL/PGK-MCS-IRES-puro
aagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgacgatggtcgagtaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctctagcgacgcgtgtcgacgatatcttcgaaggatccagtaaagcatagcggccgtaaattccgcccctctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataagcttgccacaaccatgaccgagtacaagcccacggtgcgcctcgccacccgcgacgacgtcccccgggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgactcgagggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgc

SEQ ID NO: 107 - DNA, 6475 bp, Artificial Sequence
  pT2/UASTK-d2eGFP-SV40-neo
caaggcgattaagttgggtaacgccagggttttcccagtcacgacgttgtaaaacgacggccagtgagcgcgcgtaatacgactcactatagggcgaattgcaattggaattcggatccctatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttaggtttaaaccaaggcggagtactgtcctccgggctggcggagtactgtcctccggcaaggtcggagtactgtcctccgacactagaggtcggagtactgtcctccgacggtggccgccccgactgcatctgcgtgttcaaattcgccaatgacaagacgctgggcggggtttgtgtcatcatagaactaaagacatgcaaatatatttcttccggggacaccgccagcaaacgcgagcaacgggccacggggatgaagcagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaagcttagccatggcttcccgccggaggtggaggagcaggatgatggcacgctgcccatgtcttgtgcccaggagagcgggatggaccgtcaccctgcagcctgtgcttctgctaggatcaatgtgtagatgcgcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttgtttaaaccccacaataagttgggtgaattttggctcattcctcctttctataggattgaggtcagagctttgtgatgggaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttggatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctggatcccttgaaatacatccacaggtacacctccaattgactcaaatgatgtcaattagtctatcaggagctctttaagcttgtggaaggctactcgaaatgtttgacccaagttaaacaatttaaaggcaatgctaccaaatactaattgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtatagggatcctctagctagagtcgacctcgagggggggcccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctg

SEQ ID NO: 108 - DNA, 7726 bp, Artificial Sequence
  pCCL-PGK-mCherry
aagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgacgatggtcgagtaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctctagcgggatccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagaattgactcgagggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgc

SEQ ID NO: 109 - DNA, 10881 bp, Artificial Sequence
  pCCL/PGK-FahMut-P2A-eGFP-IRES-puro
aagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgacgatggtcgagtaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctctagcgaatgtcctttattccagtggccgaggactccgactttcccatccaaaacctgccctatggtgttttctccactcaaagcaacccaaagccacggattggtgtagccatcggtgaccagatcttggacctgagtgtcattaaacacctctttaccggacctgccctttccaaacatcaacatgtcttcgatgagacaactctcaataacttcatgggtctgggtcaagctgcatggaaggaggcaagagcatccttacagaacttactgtctgccagccaagcccggctcagagatgacaaggagcttcggcagcgtgcattcacctcccaggcttctgcgacaatgcaccttcctgctaccataggagactacacggacttctactcttctcggcagcatgccaccaatgttggcattatgttcagaggcaaggagaatgcgctgttgccaaattggctccacttacctgtgggataccatggccgagcttcctccattgtggtatctggaaccccgattcgaagacccatggggcagatgagacctgataactcaaagcctcctgtgtatggtgcctgcagactcttagacatggagttggaaatggctttcttcgtaggccctgggaacagattcggagagccaatccccatttccaaagcccatgaacacattttcgggatggtcctcatgaacgactggagcagtaatgcctggtggcccagcttcctctgatgttctgttcttaggggcacacacaggagttgggtatgggacaggaggcctaagtactacaggggtgataccatgcagacttctgactctgtgggtgtggggcagtcacagcttccctgagtagctttctcataagtggaaggatggagctgacagaacctaaagctttatcaagccctacacactccactcactgttgccagcaccatcagctgctgatgaagagctgtacatggagggattttagtcagtggctgaattgagccttactattcatcagcccagcatctgagtcccagctctacaccctgataccatgtgtaagaggaagtgggtccagaaatctcaggagagggccttcccccattcaggcccttggtagggactgtcctgtcatccttcattgttccctagcacagcacctccaaggacctctgtcttcatactgaatacctttctgtttgttccctgggagatctggttggccagagccatgtagagaggtccatgtaggtcaaggacaggaagcagcttggagtgagtctgtaagccacggtgagacctaagcttgctgtcttttcatagcacgagacatccagcaatgggagtacgtcccacttgggccattcctggggaaaagctttggaaccacaatctccccgtgggtggtgcctatggatgccctcatgccctttgtggtgccaaacccaaagcaggaccccaagcccttgccatatctctgccacagccagccctacacatttgatatcaacctgtctgtctctttgaaaggagaaggaatgagccaggcggctaccatctgcaggtctaactttaagcacatgtactggaccatgctgcagcaactcacacaccactctgttaatggatgcaacctgagacctggggacctcttggcttctggaaccatcagtggatcagaccctgaaagctttggctccatgctggaactgtcctggaagggaacaaaggccatcgatgtggagcaggggcagaccaggaccttcctgctggacggcgatgaagtcatcataacaggtcactgccagggggacggctaccgtgttggctttggccagtgtgctgggaaagtgctgcctgccctttcaccagccggatccggcgcaacaaacttctctctgctgaaacaagccggagatgtcgaagagaatcctggaccggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggcagtaaagcatagcggccgtaaattccgcccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataagcttgccacaaccatgaccgagtacaagcccacggtgcgcctcgccacccgcgacgacgtcccccgggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgactcgagggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgc

SEQ ID NO: 110 - DNA, 20 bp, Artificial Sequence
  VEGFA site 1 - sequence
ctcctggaagatgtccacca

SEQ ID NO: 111 - DNA, 21 bp, Artificial Sequence
  VEGFA site 1 - Fw primer
gagcccagggagcaaaggtca

SEQ ID NO: 112 - DNA, 24 bp, Artificial Sequence
  VEGFA site 1 - Rv primer
ggttcctggttgtgcttagtggga

SEQ ID NO: 113 - RNA, 116 nt, Artificial Sequence
  sgRNA2.1 VEGFA(site 1)
cuccuggaagauguccaccaguuuaagagcuaugcuggaaacagcauagcaaguuuaaauaaggcuaguccguuaucaacuuugcuggaaacagcaaaguggcaccgagucggugc

(19) United States
(12) Patent Application Publication
(10) Pub. No.: US 2024/0366790 A1
(43) Pub. Date: Nov. 7, 2024

(54) LENTIVIRUS-DERIVED NANOPARTICLES COMPRISING CRISPR/CAS9 RIBONUCLEOPROTEIN COMPLEXES

(71) Applicant: AARHUS UNIVERSITET, Aarhus C (DK)

(72) Inventors: Mads Valdemar ANDERSON, Aarhus C (DK); Jacob Giehm MIKKELSEN, Aarhus C (DK); Kristian Alsbjerg SKIPPER, Aarhus C (DK); Emil Aagaard THOMSEN, Aarhus C (DK); Sofie ANDERSEN, Aarhus C (DK); Jakob Haldrup JENSEN, Aarhus C (DK)

(21) Appl. No.: 18/562,938

(22) PCT Filed: May 20, 2022
(86) PCT No.: PCT/EP2022/063786

§ 371 Date: Nov. 21, 2023

(30) Foreign Application Priority Data
May 21, 2021 (EP) 21175250.6

(51) Int. Cl. A61K 48/00; A61P 27/02; C12N 9/22; C12N 15/11; C12N 15/86

(52) U.S. Cl. CPC A61K 48/005; A61P 27/02; C12N 9/22; C12N 15/111; C12N 15/86; C12N 2310/20; C12N 2740/16023; C12N 2740/16041; C12N 2740/16052

(57) ABSTRACT

The present invention relates to a lentivirus-derived particle comprising one or more Cas9-like proteins and at least one optimized sgRNA, wherein the optimized sgRNA comprises a targeting region and a non-targeting region, wherein said non-targeting region comprises a nucleotide sequence corresponding to SEQ ID NO: 1 or sequences having at least 90% sequence identity to SEQ ID NO: 1, said nucleotide sequence further comprising at least the following modifications: an extended repeat-anti-repeat region comprising a first extension of 2-8 base pairs in the repeat-anti-repeat region corresponding to nucleotides 1-12 and 17-30 of SEQ ID NO: 1; and optionally, an extended stem-loop 2 region comprising a second extension of 2-8 base pairs in the stem-loop 2 corresponding to the nucleotides 48-61 of SEQ ID NO: 1; and/or optionally, an A-U flip of the nucleotides corresponding to nucleotides 5 and 36 of SEQ ID NO: 1.

### TECHNICAL FIELD OF THE INVENTION

The present invention relates to lentivirus-derived nanoparticles comprising CRISPR/CAS9 ribonucleoprotein (RNP). In particular, the present invention relates to optimised lentivirus-derived nanoparticles having improved potency of RNP delivery and increased efficiency.

### BACKGROUND OF THE INVENTION

CRISPR/Cas9-mediated somatic cell genome editing is showing promise to cure inherited and infectious disease, and can potentially transform the lives of patients suffering from severe diseases. Efficient genome editing can be achieved by DNA-free delivery of CRISPR/Cas9 ribonucleoprotein (RNPs) complexes consisting of recombinant Cas9 protein complexed with synthetic and chemically modified single guide RNAs (sgRNAs). Transient exposure to the RNA-guided nucleases delivered to cells by RNP nucleofection has already shown potential for ex vivo correction of hematopoietic stem cells. However, for potential in vivo use directly in patients, the delivery of RNP genome editing complexes is challenged by strong limitations of physical or chemical methods for delivery. Based on the wide use of virus-based vectors in conventional gene therapies, viral vectors carrying gene expression cassettes encoding Cas9 and sgRNA have been adapted for CRISPR/Cas delivery for both in vitro and in vivo use. However, although AAV-mediated delivery typically results in potent gene delivery leading to robust Cas9-directed DNA cleavage, this delivery platform is challenged by size limitations that prevent packaging of Streptococcus pyogenes Cas9 (SpCas9) and sgRNA expression cassettes in a single vector. Also, episomal AAV vector-derived DNA intermediates are retained for a long period of time in non-dividing cells and may even integrate into the double-stranded break. This may result in both high and prolonged expression of Cas9 leading potentially to unspecific DNA cleavage and/or depletion of gene-corrected cells by the immune system.

In order to achieve transient exposure to Cas9 in vivo, non-viral nanoparticles have been developed. Gold-based nanoparticles have been developed as carriers of Cas9/sgRNA-containing RNPs in muscle and brain tissue, but such materials may accumulate in the liver inducing acute inflammation and cellular damage. Furthermore, lipid-based nanoparticles have been demonstrated to deliver RNPs in vivo to modify inner hair cells, albeit at a low efficiency. Vesicles coated with the envelope from vesicular stomatitis virus (VSV-G) have been engineered to deliver Cas9 and sgRNA. However, the delivery of particles carrying surface VSV-G protein, including lentivirus-based vectors, may be hampered in clinical settings by inactivation of human serum complement.

Retro- and lentivirus-derived virus particles can be engineered to transiently deliver foreign proteins, including DNA transposases, zinc-finger nucleases, and TAL-effector nucleases. Also, Cas9 protein and sgRNAs can be incorporated in such particles, which have been produced through (i) fusion of Cas9 to Gag/GagPol polyproteins, (ii) introduction of aptamers into the stem-loops of the sgRNA sequence and fusion of aptamer-binding proteins to Gag/GagPol and (iii) fusion of Cas9 to the accessory protein Vpr. Such virus-derived particles uniquely combine the transient delivery of Cas9 protein potentially complexed with sgRNA with the inherent fusogenic properties of virus particles and their ability to transport enzymatic protein into target cells. Whereas such vectors are generally pseudotyped with the vesicular stomatitis virus glycoprotein (VSV-G), which facilitates a broad tropism, alternative surface envelope proteins can be incorporated into the particles to restrict cargo delivery to different cell types and to avoid inactivation in human serum. This ability to target specific cell types with Cas9/sgRNA-loaded virus particles may potentially allow genetic intervention in a targeted population of cells only, which may be crucial for effective and safe in vivo delivery of genome-editing tool kits. To comply with clinical translation, virus-derived protein and RNA delivery methods need to be further optimized, allowing dosages to be low and activity to be targeted to relevant cells.

Hence, an improved method for genome-editing would be advantageous, and in particular more efficient and/or reliable virus particles, which would allow dosages to be low, would be advantageous.

### SUMMARY OF THE INVENTION

Thus, an object of the present invention relates to the provision of efficient and reliable virus particles, which can be used for effective genome-editing even at low doses.

It is demonstrated that sgRNAs are incorporated into Cas9-loaded lentivirus-derived nanoparticles (LVNPs) in a Cas9-dependent manner. Improved targeted DNA cleavage rates in cells treated with LVNPs loaded with RNPs carrying scaffold-optimized sgRNAs are shown in the examples. Thus, the virus particles are efficiently adapted for transient delivery of CRISPR kits for genome editing.

As further demonstrated by the examples, targeted gene disruption can be robustly achieved in cells exposed to lentiviral particles loaded with RNP complexes preassembled in virus-producing cells and show improved targeted DNA cleavage with effectively incorporated scaffold-optimized sgRNAs. The results further demonstrate an increased affinity between Cas9 and the sgRNA. This may result in a more stable RNP complex in recipient cells facilitating an increased proportion of Cas9 reaching the nucleus complexed with the sgRNA resulting in higher levels of targeted DNA cleavage using the LVNP technology.

Thus, one aspect of the invention relates to a lentivirus-derived particle comprising one or more Cas9-like proteins and at least one optimised sgRNA, wherein the optimized sgRNA comprises a targeting region and a non-targeting region, wherein said non-targeting region comprises a nucleotide sequence corresponding to SEQ ID NO: 1 or sequences having at least 90% sequence identity to SEQ ID NO: 1, said nucleotide sequence further comprising at least the following modifications
an extended repeat-anti-repeat region comprising a first extension of 2-8 base pairs in the repeat-anti-repeat region corresponding to nucleotides 1-12 and 17-30 of SEQ ID NO: 1;
optionally, an extended stem-loop 2 region comprising a second extension of 2-8 base pairs in the stem-loop 2 corresponding to the nucleotides 48-61 of SEQ ID NO: 1; and/or
optionally, an A-U flip of the nucleotides corresponding to nucleotides 5 and 36 of SEQ ID NO: 1.

Another aspect of the present invention relates to a composition comprising a lentivirus-derived particle or a plurality of LVNPs as described herein.

Yet another aspect of the present invention is to provide a method of producing a lentivirus-derived particle as described herein, wherein said method comprises the steps of
a) providing packaging plasmid(s) including a packaging plasmid comprising at least gagpol viral genes under the control of a promoter;
b) providing a nucleic acid capable of transcribing at least one sgRNA;
c) transfecting said packaging plasmid(s) and said nucleic acid constructs into a producer cell;
d) purifying lentivirus-derived particles from said producer cell.

An even further aspect of the present invention relates to an in vitro use of the lentivirus-derived particle as described herein or produced by a method as described herein, and/or a composition as described herein for genome engineering or cell engineering.

### BRIEF DESCRIPTION OF THE FIGURES

FIG. 1 shows packaging of SpCas9 into integrase-defective lentiviral vectors (IDLVs). (A) Schematic representation of the plasmids encoding the N-terminal and C-terminal fusion constructs (top) and the production of SpCas9-loaded IDLVs (bottom). In the N-terminal fusion construct Gag is composed of SpCas9, the phospholipase C-δ1 pleckstrin homology domain (PH), matrix (MA), capsid (CA), nucleocapsid (NC) and p6, and Pol is composed of protease (PR), reverse transcriptase (RT) and integrase with the D64V mutation (IN). In the C-terminal fusion construct Gag is composed of MA, CA, NC and p6, and Pol is composed of PR, RT, IN, and SpCas9. IDLVs were produced by co-transfecting HEK293T cells with pMD2.G (3), pRSV-Rev (4), and pCCL-PGK-eGFP (5 in combination with either pMDLg/p-RRE-D64V-PCS-SpCas9 (1) or pSpCas9-PH-gagpol-D64V and pMDLg/p-RRE-D64V (2). (B) Analysis of the IDLV contents by Western blot using FLAG- and p24-specific antibodies. Left panel shows IDLVs produced with N-terminal SpCas9 fusion (Mat-SpCas9 format), and the right panel IDLVs produced with C-terminal SpCas9 fusion (Int-SpCas9 format). Each panel contains two lanes of IDLVs produced with SpCas9 constructs, two lanes with IDLVs produced without SpCas9 constructs and one lane with a transfection control. SQV indicates whether the virus was produced in presence of the protease inhibitor saquinavir. Representative results of two or more independent experiments done on different days. (C) Estimation of transduction efficiencies from SpCas9-loaded IDLVs. IDLVs loaded with N-terminal or C-terminal SpCas9 were packaged with an eGFP expression vector, and titers were estimated by flow cytometry. N-terminal fusions were packaged with increasing amounts of wild-type Gag/GagPol-D64V as indicated. A regular IDLV and IDLV produced with reduced GagPol plasmids were included as controls. n=2 independent experiments done on different days.

FIG. 2 shows genome editing with SpCas9-loaded IDLVs and LVNPs. (A) Schematic representation of sgRNA delivery methods based on delivery of lentiviral vector encoding the sgRNA (left) or incorporation of sgRNA expressed in producer cells in virus particles. SpCas9-loaded IDLVs and LVNPs were produced in HEK293T cells by co-transfection as shown in FIG. 2A. For packaging of sgRNA-encoding vector with the exception that the EGFP encoding transfer plasmid was replaced with a sgRNA encoding transfer plasmid (IDLVs) or sgRNA-encoding plasmid for expression of sgRNAs in the producer cells (LVNPs). (B) Schematic representation of the plasmids used for sgRNA delivery in the delivery methods in A. SgRNA was encoded by a lentiGuide-puro transfer plasmid (left) or expressed on a plasmid also encoding eGFP under the control of a CBh promoter (right). (C-D) Genome editing with SpCas9-loaded IDLVs and LVNPs. 2×10⁵ HEK293T cells were reverse transduced with 300 ng p24 SpCas9-loaded IDLVs (C) or LVNPs (D). IDLVs and LVNPs produced without the VSV-G envelope protein served as negative controls. n=1-2 independent experiments done on different days.

FIG. 3 shows modified sgRNAs result in improved genome editing. (A) Schematics of the wild-type SpCas9 crRNA/tracrRNA and three different sgRNA versions. Modifications to the original (sgRNA1) scaffold are shown in bold. (B) Comparison of sgRNA1 and sgRNA2.0 at four different loci in HEK293T or HEK293-eGFPmut cells. 2×10⁵ cells were reverse transduced with 300 ng p24. (C-D) Comparison of Mat-SpCas9 and Int-SpCas9 LVNPs produced with sgRNA1, sgRNA2.0, and sgRNA2.1 targeting the AFF1 locus. 2×10⁴ 293T cells were reverse transduced with 15 ng p24 (C). 2×10⁵ 293T cells were reverse transduced with 18.75-150 ng p24 (D). (E) Comparison of Mat-SpCas9 LVNPs produced with sgRNA1, sgRNA2.0, and sgRNA2.1 targeting the Pcsk9 locus in AML12 cells. 2×10⁵ AML12 cells were reverse transduced with 18.75-150 ng p24. (F) 2×10⁴ AML12 cells were reverse transduced with 15 ng p24 and lysed after 4-96 hours. n=1 (B, D), n=2 (E), or n=3 (C, F) independent experiments done on different days.

FIG. 4 shows SpCas9 dependent sgRNA packaging into LVNPs. (A) Schematic showing the three sgRNA backbones (without 3′ uracils) with a targeting region towards AFF1, with the differences marked in grey. A spacer specific forward primer can be used in combination with the universal reverse primer and probe for all three sgRNA backbones. The area for attachment of the primers and probes on the sgRNAs are indicated above the sequences of the sgRNA scaffolds. (B-D) Quantification of sgRNA copies in LVNPs. (B) Quantification of sgRNA2.1 levels in Matrix-fused LVNPs loaded with SpCas9 (left column) or a zinc-finger nuclease (middle column). SpCas9 loaded LVNPs produced with a promoter-less sgRNA2.1 encoding plasmid served as negative control (right column). (C) Quantification of sgRNA1, sgRNA2.0, and sgRNA2.1 levels in Mat-SpCas9 and Int-SpCas9 LVNPs. (D) Quantification of sgRNA2.1 levels in LVNPs produced with reducing amounts of sgRNA encoding plasmid. (E-G) 1-D plots of quantifications in B-D. The dots above the horizontal line set at an amplitude of 2700 indicate droplets with amplified template, i.e. positive droplets, whereas dots below the line indicate droplets without amplified template, i.e. negative droplets. (H) Genome editing in HEK293T cells treated with LVNPs produced with sgRNA2.1 expressed on a separate plasmid or from the Mat-SpCas9 encoding packaging plasmid.

FIG. 5 shows incorporation of SpCas9 into lentivirus-derived nanoparticles (LVNPs). (A) Schematic representation of lentivirus (LV), LVNP, and LVNP loaded with a vector genome (LVNP/Transfer vector). (B) Top: Schematics of Int-SpCas9-LVNP composed of FLAG-Tagged SpCas9 fused to the C-terminal of the integrase domain (encoded by Pol) and flanked by a PCS for HIV-1 proteolytic release. Bottom: Schematics of Mat-SpCas9-LVNP composed of FLAG-Tagged SpCas9 fused to the N-terminal of Gag harboring an intervening phospholipase C-δ1 pleckstrin homology (PH) domain. (C) Western Blot analysis of FLAG-tagged SpCas9 (FLAG antibody) and p24 loading control of purified Int-SpCas9-LVNP (90 ng p24) and IDLV (90 ng p24) in the presence/absence of the HIV-1 protease inhibitor saquinavir (SQV). (D) The functional titer of IDLV/PGK-eGFP and Int-SpCas9-LVNP/PGK-eGFP was evaluated by flow cytometry. (E) Indel frequencies in the AFF1 locus after Int-SpCas9-LVNP transduction in a dose-escalating manner, and (F) devoid of VSV-G pseudotyping (90 ng p24). (G) Western Blotting of purified Mat-SpCas9-LVNP as described in C) using equal amounts of virus (90 ng p24) (H) Estimation of functional titer of Mat-SpCas9-LVNP with/without titration of increasing amounts of pGagPol-D64V. (I+J) Indel frequencies in the AFF1 locus after Mat-SpCas9-LVNP transduction in a dose-escalating manner, and devoid of VSV-G pseudotyping (90 ng p24). All indel frequencies and functional titers are presented as the ±SD of triplicates.

FIG. 6 shows incorporation of scaffold modified sgRNAs. (A-C) Heatmaps representing the indel frequency corresponding to the indicated p24 dose for sgRNA1, sgRNA2.0, and sgRNA2.1 for (A) Pcsk9 in AML12 hepatocytes, (B) Vegfa (site 1) in transgenic HEK293T-Vegfa cells, and (C) SERPING1 in HEK293T. (D-E) The sgRNA abundance was determined by digital droplet PCR (ddPCR) in purified Mat-SpCas9-LVNPs loaded with indicated scaffold for (D) Pcsk9, and (E) Vegfa (site 1). (F) The sgRNA abundance in purified Mat-SpCas9-LVNPs produced with/without inclusion of the U6 promoter in the sgRNA expression backbone and in LVNPs loaded with Zink Finger Nucleases (ZFN) instead of SpCas9. Significant p-values (Mann-Whitney U-test) are marked by *p<0.05 and presented as ±SD of triplicates.

FIG. 7 shows refinement of LVNP content. (A) The optimal Mat-SpCas9-LVNP stoichiometry was evaluated by titration of increasing amounts of pGagPol-D64V vs. pMat-SpCas9 (13 μg plasmid in total) in producer cells to maximize viral production. The concentration (μg/mL) was determined by p24 ELISA after purification and resuspension in equal volumes of buffer. (B) The corresponding indel frequencies following transduction of transgenic HEK293T-Vegfa cells (60 and 15 ng p24). (C) Determination of sgRNA abundance in purified Mat-SpCas9-LVNPs produced by titration of increasing amounts of pCCL-PGK-eGFP (transfer vector) vs. sgRNA expression plasmid (13 μg plasmid in total). (D) The corresponding indel frequencies by Sanger sequencing, and (E) percentage of eGFP+HEK293T-Vegfa cells by flow cytometry after transduction of the in C) produced Mat-SpCas9-LVNP (60 ng and 15 ng p24). (F) The performance of Mat-SpCas9-LVNP across multiple loci and cell lines as determined by indel frequencies and percentage of eGFP+ cells. Significant p-values (Mann-Whitney U-test) are marked by *p<0.05 and presented as ±SD of triplicates.

FIG. 8 shows transient genome editing reduces genotoxicity. (A) HEK293T cells were transduced (90 ng p24) with LVNP/PGK-mCherry carrying sgRNA-d2eGFP or LV/PGK-d2eGFP-IRES-Puro and the fluorescence was evaluated by flow cytometry after three days. (B) HEK293T were transduced by LVNP/PGK-mCherry carrying sgRNA-d2eGFP and co-transduced with LV/PGK-d2eGFP-IRES-Puro at the indicated time points. Using flow cytometry, the level of d2eGFP knockout was calculated at each time point to evaluate the kinetics of transient LVNP delivery. (C) The indel frequency was measured continuously in the Pcsk9 locus in AML12 hepatocytes after Mat-SpCas9-LVNP transduction. (D) The emergence of double-stranded DNA breaks in Pcsk9 was evaluated over the course of 24 hours in pull-down experiments. The fold-enrichment was calculated by this formula: enrichment=2ΔCt (on-target region)/2ΔCt (control-region). (E) Tracking of sgRNA abundance in recipient cells by ddPCR. (F-G) monitoring of on/off-target events over the course of 14 days in the Pcsk9 locus and a well-characterized off-target locus following (F) nucleofection, and (G) LVNP2.2 transduction in AML12 hepatocytes. Significant p-values (Mann-Whitney U-test) are marked by *p<0.05 and presented as ±SD of triplicates.

FIG. 9 shows gene disruption in the murine eye. (A) Time course for in vivo evaluation of Mat-SpCas9-LVNP-directed Vegfa (site 1) disruption. (B) Subretinal injections of 2 μL (16 ng p24) Mat-SpCas9-LVNP (delivering a sgRNA targeting Vegfa (site 1) and a GFP transgene) were administered to the subretinal space in proximity to the retinal pigmental cells (RPE) cells in 8-week-old, male C57Bl/6J mice (n=9). (C) Representative gating strategy to eGFP+ and eGFP-RPE cells of Naïve or Mat-SpCas9-LVNP transduced mice. (D) The percentage of alleles with ‘on-target’ disruption of Vegfa (site 1), and (E) ‘off-target’ disruption in two well-characterized loci by ICE analysis. Data is presented as ±SD of technical triplicates.

The present invention will now be described in more detail in the following.

### DETAILED DESCRIPTION OF THE INVENTION

### Definitions

Prior to discussing the present invention in further details, the following terms and conventions will first be defined:

### Fusion Gene

In the present context, the term “fusion gene” refers to a hybrid gene formed from two or more previously separated genes.

### Fusion Protein

In the present context, the term “fusion protein” refers to a hybrid protein formed from combination of two or more proteins.

### CRISPR/Cas9

In the present context, the term “CRISPR/Cas9” refers to an RNA-guided targeted genome editing tool allowing e.g. for gene knockout, knock-in, insertions and deletions in cell lines and animals. The CRISPR/Cas9 genome editing system requires two components, Cas9, the RNA-guided endonuclease, and a guide RNA (gRNA); the gRNA guides Cas9 to the location in the genome sequence specifically through base pairing between the gRNA and the targeted DNA sequence. Targeted binding of the Cas9/gRNA complex leads to formation of a double-stranded break in the DNA at a position strictly dictated by interaction between gRNA and target DNA.

### Indels

In the present context, the term “indels” refers to insertion-deletion mutations and is possibly formed when repair of the DNA is performed by non-homologous end-joining (NHEJ). These are likely to disrupt the reading frame if the cut site is located within the coding region of a gene.

sgRNA

In the present context, the term “sgRNA” refers to a single guide RNA, which enables highly efficient and accurate editing. “gRNA” is used interchangeably with “sgRNA”. The sgRNA comprises two parts—a targeting region and a non-targeting region. The sgRNA may further comprise a variable number of uracil nucleotides at the 3′end. The number of uracil nucleotides depend on expression system used.

In one embodiment, said non-targeting region is downstream of said targeting region. Hereby, is to be understood that the non-targeting region is positioned at the 3′ end of the targeting region.

It should be understood that the “targeting sequence” relates to the part of the sgRNAs, which is complementary to the “target site” in the genome. The exact length of the targeting region in the sgRNAs may vary. Thus, in one embodiment, the targeting region has a length of 17-24 nucleotides, such as 18-22, like 19-21, such as 20 nucleotides.

### Non-Targeting Region

In the present context, the term “non-targeting region” refers to the part of the sgRNA involved in the interaction with the Cas9 and the binding hereto.

In one embodiment, such as when designed for functioning with Cas9, the non-targeting region is positioned downstream of the targeting region i.e. reading from the 5′ end of the sgRNA, the targeting region is followed by the non-targeting region.

The non-targeting region of the sgRNA may further be divided into areas relating to the secondary structure of the sgRNA i.e. a “repeat region”, “anti-repeat region”, “tetraloop”, “stem loop1”, stem loop2” and “stem loop3”. The repeat and anti-repeat regions base pair forming a repeat-anti-repeat duplex. As an example, sgRNA1 non-targeting region (SEQ ID NO: 1): repeat region (nucleotides 1-12), tetraloop (nucleotides 13-16), anti-repeat region (nucleotides 17-30), single nucleotide (nucleotide 31), stem loop 1 (nucleotides 32-42), linker (nucleotides 43-47), stem loop 2 (nucleotides 48-61), stem loop 3 (nucleotides 62-76).

### Sequence Identity

In the present context, the term “sequence identity” is here defined as the sequence identity between genes or proteins at the nucleotide, base or amino acid level, respectively. Specifically, a DNA and a RNA sequence are considered identical if the transcript of the DNA sequence can be transcribed to the identical RNA sequence.

Thus, in the present context “sequence identity” is a measure of identity between proteins at the amino acid level and a measure of identity between nucleic acids at nucleotide level. The protein sequence identity may be determined by comparing the amino acid sequence in a given position in each sequence when the sequences are aligned. Similarly, the nucleic acid sequence identity may be determined by comparing the nucleotide sequence in a given position in each sequence when the sequences are aligned.

To determine the percent identity of two amino acid sequences or of two nucleic acids, the sequences are aligned for optimal comparison purposes (e.g., gaps may be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment, the two sequences are the same length.

In another embodiment, the two sequences are of different length and gaps are seen as different positions. One may manually align the sequences and count the number of identical amino acids. Alternatively, alignment of two sequences for the determination of percent identity may be accomplished using a mathematical algorithm. Such an algorithm is incorporated into the NBLAST and XBLAST programs of (Altschul et al. 1990). BLAST nucleotide searches may be performed with the NBLAST program, score=100, wordlength=12, to obtain nucleotide sequences homologous to a nucleic acid molecules of the invention. BLAST protein searches may be performed with the XBLAST program, score=50, wordlength=3 to obtain amino acid sequences homologous to a protein molecule of the invention.

To obtain gapped alignments for comparison purposes, Gapped BLAST may be utilized. Alternatively, PSI-Blast may be used to perform an iterated search, which detects distant relationships between molecules. When utilizing the NBLAST, XBLAST, and Gapped BLAST programs, the default parameters of the respective programs may be used. See http://www.ncbi.nlm.nih.gov. Alternatively, sequence identity may be calculated after the sequences have been aligned e.g. by the BLAST program in the EMBL database (www.ncbi.nlm.gov/cgi-bin/BLAST). Generally, the default settings with respect to e.g. “scoring matrix” and “gap penalty” may be used for alignment. In the context of the present invention, the BLASTN and PSI BLAST default settings may be advantageous.

The percent identity between two sequences may be determined using techniques similar to those described above, with or without allowing gaps. In calculating percent identity, only exact matches are counted. An embodiment of the present invention thus relates to sequences of the present invention that has some degree of sequence variation.

### Base Pair

In the present context, the term “base pair” refers to two nucleotides being arranged in a secondary structure in a way enabling the two nucleotides to perform a base pairing.

### Base Pairing

In the present context, when referring to a “base pairing”, G pairs to C, A pairs to T and U and vice versa. In some embodiments, G may also pair to U and vice versa to form a so-called wobble base pair. A wobble base pair is a non-Watson-Crick base pairing between two nucleotides in RNA molecules. The four main wobble base pairs are guanine-uracil, inosine-uracil, inosine-adenine, and inosine-cytosine (G-U, I-U, I-A and I-C).

### Substitutions

In the present context, the term “substitutions” refers to exchange of one nucleotide with a different nucleotide.

### Modifications

In the present context, the term “modifications” refers to the change of a sequence by substitution of nucleotide(s), deletion of nucleotide(s) and/or insertion of nucleotide(s).

### A-U Flip

In the present context, the term “A-U flip” refers to a base pair being adenosine (A) and urasil (U), where the adenosine (A) is substituted with an uracil (U) and the uracil (U) is substituted with an adenosine (A). Hereby, the base pairing is maintained but the position of the two nucleotides is exchanged.

### Extension

In the present context, the term “extension” refers to the elongation of a secondary structure of the sgRNA.

### Knockout

In the present context, the term “knockout” refers to the removal of a gene, part of a gene or disruption of the reading frame for the gene whereby the gene is no longer correctly transcribed or translated.

### Knock-In

In the present context, the term “knock-in” refers to the addition into or repair of a gene in the genome of a cell or an animal. This may be obtained by adding donor DNA during the homologous recombination.

### Donor or Donor Sequence

In the present context, the terms “donor” or “donor sequence” refer to a DNA sequence to be introduced into the genome at the double-stranded cut performed by the CRISPR/Cas complex. A donor sequence contains two stretches of DNA. In one embodiment, the donor sequence contains two stretches of DNA with homology to regions flanking the genomic cut site. Administration of the donor sequence allows the cut sit to be repaired by homologous recombination.

### Homology Arms

In the present context, the term “homology arms” refers to a left (LHA) and right (RHA) arranged on either side of the donor sequence to be inserted. The homology arms are designed to flank the Cas9 cleavage site.

### Lentivirus-Derived Particle

In the present context, the term “lentivirus-derived particle” is used interchangeably with LVNP. “LVNP” is short for Lentivirus-derived NanoParticle. LVNP is a virus-like particle derived from lentivirus. The LVNP is capable of introducing proteins and nucleic acids/nucleotides to a cell by transduction but is not capable of integrating and replicating itself in the host.

In one embodiment, the LVNP would comprise features from the lentivirus, which enables it to deliver proteins and nucleic acids/nucleotides to the cell such as the Gag polyprotein resulting in Matrix, Capsid and Nucleocapsid proteins and the protease from the Pol polyprotein.

### Lentiviral Vector System

In the present context, the term “lentiviral vector system” refers to a system comprising packaging plasmids and potentially one or more transfer plasmid(s). The packaging plasmids encode the necessary components for vector production and the transfer plasmid carries gene(s) of interest such as sgRNA or donor sequences.

### Cell-Efficient Amount

In the present context, the term “cell-efficient amount” refers to the amount efficient for a particular cell type for be modified with an indel formation of at least 40%, such as at least 50%, like at least 60%, such as at least 70%, like at least 80%, such as at least 90%, like at least 95%, such as at least 98%, like at least 100%.

### Lentivirus-Derived Particle

In one aspect, the invention relates to a lentivirus-derived particle comprising one or more Cas9-like proteins and at least one optimized sgRNA, wherein the optimized sgRNA comprises a targeting region and a non-targeting region, wherein said non-targeting region comprises a nucleotide sequence corresponding to SEQ ID NO: 1 or sequences having at least 90% sequence identity to SEQ ID NO: 1, said nucleotide sequence further comprising at least the following modifications
an extended repeat-anti-repeat region comprising a first extension of 2-8 base pairs in the repeat-anti-repeat region corresponding to nucleotides 1-12 and 17-30 of SEQ ID NO: 1;
optionally, an extended stem-loop 2 region comprising a second extension of 2-8 base pairs in the stem-loop 2 corresponding to the nucleotides 48-61 of SEQ ID NO: 1; and/or
optionally, an A-U flip of the nucleotides corresponding to nucleotides 5 and 36 of SEQ ID NO: 1.

In another aspect, the invention relates to a lentivirus-derived particle comprising one or more Cas9-like proteins and at least one optimized sgRNA, wherein the optimized sgRNA comprises a targeting region and a non-targeting region, wherein said non-targeting region comprises a nucleotide sequence corresponding to SEQ ID NO: 1 or sequences having at least 90% sequence identity to SEQ ID NO: 1, said nucleotide sequence comprising at least the following modifications
an extended repeat-anti-repeat region comprising a first extension of 2-8 base pairs in the repeat-anti-repeat region corresponding to nucleotides 1-12 and 17-30 of SEQ ID NO: 1;
optionally, an extended stem-loop 2 region comprising a second extension of 2-8 base pairs in the stem-loop 2 corresponding to the nucleotides 48-61 of SEQ ID NO: 1; and/or
optionally, an A-U flip of the nucleotides corresponding to nucleotides 5 and 36 of SEQ ID NO: 1.

Accordingly, the lentivirus-derived particle comprises both a Cas9-like protein and one or more optimised sgRNAs. The sgRNAs are optimized in their non-targeting region i.e. the non-targeting region has at least 90% sequence identity to SEQ ID NO: 1 and furthermore comprises a modification being an extended repeat-anti-repeat region as well as potentially, even further modifications as an extended stem-loop 2 region and/or an A-U flip.

In a further aspect, the invention relates to a lentivirus-derived particle comprising one or more Cas9-like proteins and at least one optimized sgRNA, wherein the optimized sgRNA comprises a targeting region and a non-targeting region, wherein said non-targeting region comprises or consists of a nucleotide sequence corresponding to SEQ ID NO: 2 or SEQ ID NO: 3, or sequences having at least 90% sequence identity to SEQ ID NO: 2 or SEQ ID NO: 3.

In one embodiment, said particle is an integrase-defective lentivirus. This is formed by using a packaging plasmid in which the integrase is mutated to obtain a defective integrase. This plasmid is also referred to as an integrase-defective lentiviral vector. In one embodiment, the integrase comprises a D64V mutation as e.g. the integrase encoded by the plasmid pSpCas9-PH-gagpol-D64V (SEQ ID NO: 54).

The LVNP may be altered by pseudotyping as commonly known in the art. This results in the LVNPs bearing glycoproteins from other enveloped viruses, whereby the LVNP inherits the tropism from the virus from which the glycoprotein is derived. In a further embodiment, said particle is a VSV-G-pseudotyped lentivirus-derived particle. Hereby, is to be understood that the lentivirus-derived particle includes vesicular stomatitis virus glycoprotein (VSV-G) as an envelope protein enabling the lentiviral vector to enter multiple cells via the LDL-Receptor family.

The efficiency of the LVNPs as described herein may be further optimised by ensuring that the sgRNA and Cas9 are provided in the LVNP in a certain ratio. In one embodiment, the ratio between sgRNA:Cas9 is 2:1-1:2, such as around 1:1.

As demonstrated by the examples, LVNPs according to the present invention is highly efficient in their capacity of genome modification. Hence in one embodiment, the lentivirus-derived particle has an indel formation of at least 40%, like at least 50%, such as at least 60%, like at least 70%, such as at least 80%, like at least 90%, such as at least 95%, like at least 98%, such as at least 99%.

sgRNA

The sequence of the sgRNA depends on the site in the genome to be targeted by the Cas9/sgRNA complex. The at least one optimized sgRNA, wherein the optimized sgRNA comprises a targeting region and a non-targeting region, wherein said non-targeting region comprises a nucleotide sequence corresponding to SEQ ID NO: 1 or sequences having at least 90% sequence identity to SEQ ID NO: 1, said nucleotide sequence further comprising being modified with at least the following modifications
an extended repeat-anti-repeat region comprising a first extension of 2-8 base pairs in the repeat-anti-repeat region corresponding to nucleotides 1-12 and 17-30 of SEQ ID NO: 1;
optionally, an extended stem-loop 2 region comprising a second extension of 2-8 base pairs in the stem-loop 2 corresponding to the nucleotides 48-61 of SEQ ID NO: 1; and/or optionally, an A-U flip of the nucleotides corresponding to nucleotides 5 and 36 of SEQ ID NO: 1.

In one embodiment, the sequences have at least 95% sequence identity to SEQ ID NO: 1, such as at least 98% sequence identity to SEQ ID NO: 1, like at least 99% sequence identity to SEQ ID NO: 1. It is thus to be understood that the percentage sequence identity is without taking into consideration the further modifications described.

The sequence identity to SEQ ID NO: 1 may also be identified by a number of substitutions. These substitutions are preferably performed such that the secondary structure of the non-targeting region is not altered, or at least only by minor modifications. Hence, in one embodiment, the non-targeting region comprises at the most 7 substitutions compared to SEQ ID NO: 1, like at the most 6 substitutions, such as at the most 5 substitutions, like at the most 4 substitutions, such as at the most 3 substitutions, like at the most 2 substitutions or at the most 1 substitution. It is to be understood that these substitution does not include the modifications as defined. Thus, the substitutions are to be understood as in addition to the modifications.

The non-targeting region may be of a certain length, which enables the secondary structure of the non-targeting region to be maintained. Thus, in a further embodiment, the non-targeting region has a length of at the most 120 nucleotides, such as at the most 115 nucleotides, like at the most 110 nucleotides, such as at the most 108 nucleotides, like at the most 106 nucleotides, such as at the most 104 nucleotides, like at the most 102 nucleotides, such as at the most 100 nucleotides, like at the most 98 nucleotides, such as at the most 96 nucleotides, like at the most 94 nucleotides, such as at the most 92 nucleotides, like at the most 90 nucleotides, such as at the most 88 nucleotides, like at the most 86 nucleotides, such as at the most 84 nucleotides, like at the most 82 nucleotides, such as at the most 80 nucleotides.

As demonstrated by the examples, the repeat-anti-repeat region of the non-targeting region can advantageously be extended by a first extension with additional base pairs. Thus, in one embodiment, the extended repeat-anti-repeat region comprises a first extension of 3-7 base pairs, such as 4-6 base pairs, like 5 base pairs.

In a further embodiment, the first extension is inserted 3′ to nucleotide 12 and 5′ to nucleotide 17 of SEQ ID NO: 1.

In a still further embodiment, the first extension consists of the following nucleotides 5′-UGCUG-3′ (SEQ ID NO: 62) inserted 3′ to nucleotide 12 of SEQ ID NO: 1 and 5′-CAGCA-3′ (SEQ ID NO: 63) inserted 5′ to nucleotide 17 of SEQ ID NO: 1. Hereby, the nucleotides inserted 3′, base pair with the nucleotides inserted 5′ maintaining the secondary structure but extending the repeat-anti-repeat region.

As demonstrated by the examples, the stem-loop 2 region of the non-targeting region can advantageously be extended by a second extension with additional base pairs. Thus, in one embodiment, the extended stem-loop 2 region comprises a second extension of 3-7 base pairs, such as 4-6 base pairs, like 5 base pairs.

In a further embodiment, the second extension is inserted 3′ to nucleotide 52 and 5′ to nucleotide 56 of SEQ ID NO: 1.

In a still further embodiment, the second extension consists of the following nucleotides 5′-UGCUG-3′ (SEQ ID NO: 62) inserted 3′ to nucleotide 52 of SEQ ID NO: 1 and 5′-CAGCA-3′ (SEQ ID NO: 63) inserted 5′ to nucleotide 56 of SEQ ID NO: 1. Hereby, the nucleotides inserted 3′, base pair with the nucleotides inserted 5′ maintaining the secondary structure but extending the repeat-anti-repeat region.

In an even further embodiment, said nucleotide sequence comprises an A-U flip of the nucleotides corresponding to nucleotides 5 and 36 of SEQ ID NO: 1.

In a further embodiment, the non-targeting region comprises a nucleotide sequence of SEQ ID NO: 2 or 3 or sequences having at least 90% sequence identity, such as at least at least 95% sequence identity, such as at least 98% sequence identity or such as at least 99% sequence identity to SEQ ID NO: 2 or 3. Accordingly, in one embodiment, the lentivirus-derived particle comprises one or more Cas9-like proteins and optimised sgRNA, wherein the one or more optimized sgRNA comprises a non-targeting region with an extended repeat-anti-repeat region and A-U flip according to SEQ ID NO: 2 (sgRNA2.0) and/or an extended repeat-anti-repeat region, A-U flip and extended stem-loop 2 region according to SEQ ID NO: 3 (sgRNA2.1).

The non-targeting region of sgRNA2.0 (SEQ ID NO: 2) may be divided into the following secondary structure regions: repeat region (nucleotides 1-17), tetraloop (nucleotides 18-21), anti-repeat region (nucleotides 22-40), single nucleotide (nucleotide 41), stem loop 1 (nucleotides 42-52), linker (nucleotides 53-57), stem loop 2 (nucleotides 58-71), stem loop 3 (nucleotides 72-86).

The non-targeting region of sgRNA2.1 (SEQ ID NO: 3) may be divided into the following secondary structure regions: repeat region (nucleotides 1-17), tetraloop (nucleotides 18-21), anti-repeat region (nucleotides 22-40), single nucleotide (nucleotide 41), stem loop 1 (nucleotides 42-52), linker (nucleotides 53-57), stem loop 2 (nucleotides 58-81), stem loop 3 (nucleotides 82-96).

The sequence identity to SEQ ID NOS: 2 or 3 may also be identified by a number of substitutions. These substitutions are preferably performed such that the secondary structure of the non-targeting region is not altered, or at least only by minor modifications. Hence, in one embodiment, the non-targeting region comprises at the most 9 substitutions compared to SEQ ID NOS: 2 or 3, like at the most 8 substitutions, such as at the most 7 substitutions, like at the most 6 substitutions, such as at the most 5 substitutions, like at the most 4 substitutions, such as at the most 3 substitutions, like at the most 2 substitutions or at the most 1 substitution.

In a still further embodiment, the non-targeting region consists of nucleotide sequence of SEQ ID NO: 2 or 3.

The secondary structure regions stem-loop 1 and stem-loop 3 are enclosed by the Cas9 when the Cas9 and sgRNA are interacting. Thus, in one embodiment the sequence of stem-loop 1 and/or stem-loop 3 identical to the nucleotides defined by SEQ ID NOS: 1-3. Thus, an at least 90% sequence identity is with the proviso that the sequence of stem-loop 1 consists of nucleotides 32-42 of SEQ ID NO: 1, nucleotides 42-52 of SEQ ID NO: 2 or nucleotides 42-52 of SEQ ID NO: 3 and/or stem-loop 3 consists of nucleotides 62-76 of SEQ ID NO: 1, nucleotides 72-86 of SEQ ID NO: 2 or nucleotides 82-96 of SEQ ID NO: 3.

The sgRNA according to the present invention, may further comprise a number of uracil nucleotides at the 3′ end of the sgRNA. Thus, in one embodiment, the uracil nucleotides are positioned following the non-targeting region. The number of uracil nucleotides is dependent on the expression system used for the expression of the sgRNA and does not influence the functioning of the sgRNAs as described herein. Hence, in one embodiment, the sgRNA further comprises a U-region at the 3′ end comprising a variable number of the nucleotide uracil, such as 3-10 uracils, like 6 uracils.

In a further embodiment, the optimized sgRNAs comprises or consists of SEQ ID NO: 7 or SEQ ID NO: 8. Herein, the targeting region is defined by the nucleic acids “N”. It is to be understood that the length of the targeting region may vary depending to the target to be targeted. Furthermore, it is to be understood that the “N”'s may be any nucleotide, which in combination forms the sequence of the targeting region.

The targeting region is often between 17-24 nucleotides, such as 18-22 nucleotides, like 19-21 nucleotides, such as 20 nucleotides.

In one embodiment, the targeting region of the sgRNAs targets human AFF1 and the optimised sgRNAs comprise or consist of SEQ ID NO: 10 or SEQ ID NO: 11.

In one embodiment, the targeting region of the sgRNAs targets murine Pcsk9 and the optimised sgRNAs comprise or consist of SEQ ID NO: 13 or SEQ ID NO: 14.

In one embodiment, the targeting region of the sgRNAs targets serping1 and the optimised sgRNAs comprise or consist of SEQ ID NO: 16 or SEQ ID NO: 17.

In one embodiment, the targeting region of the sgRNAs targets human VEGFA (site 3) and the optimised sgRNAs comprise or consist of SEQ ID NO: 19 or SEQ ID NO: 20.

In one embodiment, the targeting region of the sgRNAs targets eGFP and the optimised sgRNAs comprise or consist of SEQ ID NO: 22 or SEQ ID NO: 23.

In one embodiment, the targeting region of the sgRNAs targets d2eGFP and the optimised sgRNAs comprise or consist of SEQ ID NO: 104.

In one embodiment, the targeting region of the sgRNAs targets Fah and the optimised sgRNAs comprise or consist of SEQ ID NO: 105.

In one embodiment, the targeting region of the sgRNAs targets human and murine VEGFA (site 1) and the optimised sgRNAs comprise or consist of SEQ ID NO: 113

The sgRNA may be expressed on a transfer plasmid or on a separate expression plasmid. Alternatively, the sgRNA may be arranged on the same plasmid as the gag/pol.

### Cas9

The Cas9-like protein included in the LVNP may be any protein capable of exerting a function as provided by Cas9. In one embodiment, the RNA guided endonuclease is selected from the group consisting of Cas9 endonucleases, including SpCas9, SaCas9, NmCas9, StCas9. In a further embodiment, said Cas9-like protein is SpCas9.

The Cas9 protein may be a non-cleavable fusion protein or a cleavable fusion protein resulting in free Cas9 protein.

The Cas9-like protein may be fused with the gag/pol proteins in order to obtain a better inclusion of the Cas9-like protein in the LVNP. In one embodiment, said Cas9-like protein is fused to the N-terminal of the GagPol polypeptide. This is referred to as Mat-Cas9 as the Cas9 is fused to the Matrix protein. In another embodiment, said Cas9-like protein is fused to the C-terminal of the GagPol polypeptide. This is referred to as Int-Cas9 as the Cas9 is fused to the Integrase. In a still further embodiment, said Cas9-like protein is fused to the N-terminal of the GagPol polypeptide or said Cas9-like protein is fused to the C-terminal of the GagPol polypeptide.

### Donor Sequence

In a further embodiment, said particle further comprises a donor sequence.

This donor sequence may be arranged on the transfer plasmid together with the sgRNA or they may be arranged on different plasmids.

By introducing the donor sequence together with the sgRNA, it can be achieved that the level of donor sequence is optimal at the time of double-stranded cleavage of the genome with the CRISPR/Cas system. This is advantageous for obtaining a higher degree of knock-in in the cells.

In a further embodiment, the donor sequence comprises homology arms. The homology arms ensure correct insertion of the donor sequences into the genome of the cell and are similar to the site targeted in the genome. The donor sequence may be flanked by a homology arm on either the left or right side of the sequence i.e. a left homology arm (LHA) or a right homology arm (RHA). However, in most embodiments the donor sequence will be flanked both by a LHA and a RHA.

During CRISPR-directed DNA repair or gene insertion, three components, (i) Cas9 endonuclease, (ii) single guide RNA (sgRNA), and (iii) donor DNA for homology-directed repair (HDR), are required.

### Composition

In a further aspect, the present invention relates to a composition comprising a lentivirus-derived particle or a plurality of LVNPs as described herein.

The plurality of LVNPs in the composition may comprise sgRNAs directed towards one target. In another embodiment, the plurality of LVNPs in the composition is directed towards different targets in the genome facilitating indel formation in different loci or the induction of specific genomic deletions.

### Method of Producing an LVNP

The virus-like particle may be formed by standard methods as known to the person skilled in the art by transfecting cells such as HEK293T or COS-1 cells with plasmids enabling the cells to form virus-like particles. Stable inducible cell lines can also be generated to produce such virus-like particles by similar methods as to the generation of lentivirus producer cell lines and commonly known to the person skilled in the art and exemplified in e.g. demonstrated in Xu et al., 2001 and Manceur et al, 2017.

In particular for the formation of virus-like particles from lentivirus i.e. LVNPs, the virus-producing cell is transfected with packaging plasmids enabling the packaging of the transfer vector RNA into a LVNP and potentially a vector RNA comprising the genes of interest. Different lentivirus systems have been developed in the state of the art and all of these may be used according to the present invention as long as they are able to generate LVNPs.

As known in the art, Gag and Gag/pol precursors are expressed from full length genomic RNA as polyproteins, which require proteolytic cleavage mediated by the retroviral protease (PR) to acquire a functional conformation. Gag is composed of at least three protein units: matrix protein (MA), capsid protein (CA) and nucleocapsid protein (NC), whereas Pol consists of retroviral protease (PR), retrotranscriptase (RT) and integrase (IN).

As an example the system may comprise three packaging plasmids and a transfer plasmid. The transfer plasmid comprises the gene to be expressed by means of a promoter inserted between LTR regions. The packaging plasmids comprises the envelope protein under the control of a promoter, a plasmid comprising the Rev viral gene under the control of a promoter and a third plasmid having the remaining viral genes including the gagpol viral genes under the control of a promoter. An example of a four plasmid system is described in Dull, T. et al. 1998.

The LVNPs are then harvested from the culture medium around one to five days after transfection such as preferably two to three days after transfection.

The transfection may be performed using calcium phosphate, electroporation or similar techniques as known to the person skilled in the art.

Another aspect of the present invention relates to a method of producing a lentivirus-derived particle as described herein, wherein said method comprises the steps of
a) providing packaging plasmid(s) including a packaging plasmid comprising at least gagpol viral genes under the control of a promoter;
b) providing a nucleic acid capable of transcribing at least one sgRNA;
c) transfecting said packaging plasmid(s) and said nucleic acid constructs into a producer cell;
d) purifying lentivirus-derived particles from said producer cell.

Another aspect of the present invention relates to a method of producing a lentivirus-derived particle as described herein, wherein said method comprises the steps of
a) providing packaging plasmid(s) including a packaging plasmid comprising at least gagpol viral genes under the control of a promoter;
b) providing a nucleic acid for transcribing at least one sgRNA;
c) transfecting said packaging plasmid(s) and said nucleic acid constructs into a producer cell;
d) purifying lentivirus-derived particles from said producer cell.

In one embodiment, said nucleic acid is included in said packaging plasmid comprising said at least gagpol viral genes. Thus, cassettes encoding the proteins gag, pol and Cas9 as well as the sgRNA are in the same plasmid and to be transfected into the virus-producing cells. As disclosed by the examples this results in an increased effectivity.

In one embodiment, said method further comprises providing a Cas9-like protein. In a further embodiment, said method further comprises providing a nucleic acid encoding for a Cas9-like protein. In an even further embodiment, said nucleic acid encoding for a Cas9-like protein is comprised in a packaging plasmid comprising gagpol viral genes, such as in fusion with the gagpol viral genes, like Mat-Cas9 or Int-Cas9.

In one embodiment, said method comprises at least two packaging plasmids being a first plasmid comprising gagpol viral genes under the control of a promoter and a second plasmid comprising a nucleic acid encoding a Cas9-like protein. In an even further embodiment, said nucleic acid encoding for a Cas9-like protein is comprised in a packaging plasmid comprising gagpol viral genes, such as in fusion with the gagpol viral genes, like Mat-Cas9 or Int-Cas9. In a further embodiment, said second plasmid comprises a nucleic acid encoding SpCas9. In an even further embodiment, said second plasmid comprises a nucleic acid encoding Mat-Cas9, such as Mat-SpCas9. In an even further embodiment, said first plasmid is an integrase-defective lentiviral vector.

In another embodiment, the ratio between said first plasmid and said second plasmid is 10:90 to 90:10, such as 20:80 to 80:20, like 30:70 to 70:30, such as 40:60 to 60:40, like 50:50, such as 60:40 to 70:30. In a further embodiment, the ratio between said first plasmid and said second plasmid is 60:40. In another preferred embodiment, the ratio between said first plasmid and said second plasmid is 70:30.

In one embodiment, said method further comprises providing a transfer plasmid. In a further embodiment, said transfer plasmid comprises a donor sequence. In a still further embodiment, said method comprises providing a transfer plasmid comprising a donor sequence and a plasmid providing sgRNA. In another embodiment, the ratio between said transfer plasmid and said plasmid providing sgRNA is 10:90 to 90:10, such as 20:80 to 80:20, like 30:70 to 70:30, such as 40:60 to 60:40, like 50:50, such as 60:40 to 70:30. In a further embodiment, the ratio between said transfer plasmid and said plasmid providing sgRNA is 60:40.

In a further embodiment, said producer cell is HEK293T cells, lentiX cells or COS-1 cells.

The amount of sgRNA and Cas9 incorporated in the LVNP is preferable around 1:1. In order to obtain an efficient LVNP, where sgRNA and Cas9 are both included in the LVNPs in an amount for effective genome editing. The ratio of the plasmids transfected into the producer cell is beneficially controlled. Thus, one embodiment of the present invention relates to a method, wherein the ratio between sgRNA plasmid:Cas9 plasmid is 5:1-1:5, such as 5:1-1:1. A further embodiment of the present invention relates to a method, wherein the ratio between sgRNA plasmid:Cas9 plasmid is 3:1-1:3, such as 3:1-1:1.

A further aspect according to the invention relates to a lentivirus-derived particle (LVNP) obtained by a method as described herein.

### Uses

Being able to amend the genome of a cell in an efficient, accurate and stable manner is of high value for research relating to knock-in and knockout models. The LVNPs as described herein are able to obtain efficient genome editing even at low doses as demonstrated by the examples. This is likely due to the fact that in the presence of Cas9, sgRNAs accumulate in LVNPs, suggesting that Cas9 and sgRNAs are packaged into LVNPs as a preassembled RNP complex suggesting that sgRNAs are recruited to Gag and GagPol polypeptides (dependent on the LVNP format) through interaction with Cas9 and dragged into the virus particles via this interaction. Upon maturation of LVNPs, Cas9/sgRNA RNP complexes are released from the polypeptides and can be recruited to the cell nucleus upon delivery in recipient cells.

In a further aspect, the present invention relates to a method of amending the genome of a cell by contacting a cell to be amended with a lentivirus-derived particle as described herein.

In a further aspect, the present invention relates to the in vitro use of the lentivirus-derived particle as described herein or produced by a method as described herein, and/or a composition as described herein for genome engineering or cell engineering.

In a further aspect, the present invention relates to the lentivirus-derived particle as described herein or produced by a method as described herein, and/or a composition as described herein for use as a medicament.

In an even further aspect, the present invention relates to a lentivirus-derived particle as described herein or produced by a method as described herein, and/or a composition as described herein for use in the prevention, alleviation and/or treatment of diseases of the eye, such as age-related macular degeneration, Leber's congenital amaurosis, and retinitis pigmentosa.

In one embodiment, said lentivirus-derived particle is administered in a cell-efficient amount of LVNPs e.g. 1-1000 ng of p24 protein per 2×10⁵ recipient cells, like 2-750 ng of p24 protein per 2×10⁵ recipient cells, such as 5-500 ng of p24 protein per 2×10⁵ recipient cells, like 10-300 ng of p24 protein per 2×10⁵ recipient cells, such as 15-150 ng of p24 protein per 2×10⁵ recipient cells. The amount to be administered would be cell-line dependent and thus, would differ depending on the cell line or cell type in which the genome is to be amended.

In a further embodiment, said lentivirus-derived particle has an indel formation of at least 50%, such as at least 60%, like at least 70%, such as at least 80%, like at least 90%, such as at least 95%, like at least 98%, such as at least 99%.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1: Materials and Methods

### Cell Lines

HEK293T, HEK293-eGFPmut (Cai Y et al, 2014a), and AML12 cells were cultured in DMEM high-glucose (Sigma-Aldrich, St. Louis, MO, USA) supplemented with 5% FBS, 100 U/ml penicillin, and 100 μg/ml streptomycin, cells were split when 80-90% confluent.

### Plasmid Construction

pX330-U6-Chimeric_BB-CBh-hSpCas9 (Addgene plasmid #42230) (Cong et al, 2013). pLentiGuide-puro (SEQ ID NO: 51) (Thomsen et al., 2020), pCCL/PGK-eGFP (SEQ ID NO: 49) (Jakobsen et al., 2009) were described previously.

For construction of pSpCas9-PH-gagpol-D64V (SEQ ID NO: 54), the flag-tagged SpCas9 sequence was amplified from pX330-U6-Chimeric_BB-CBh-hSpCas9 (Addgene plasmid #42230) using MVA64+MVA65 (SEQ ID NOS: 26-27) and cloned into BshTI/Kpn2I digested pGFP-PH-gagpol-D64V (SEQ ID NO: 50).

pMDLg/p-RRE-D64V-PCS-SpCas9 (SEQ ID NO: 52) was constructed by amplification of SpCas9 sequence from pX330-U6-Chimeric_BB-CBh-hSpCas9 (Addgene plasmid #42230) using MVA9+MVA10 (SEQ ID NOS: 24+25), and amplification of a C-terminal GagPol fragment from pMDLg/p-PCS-hyPBase (SEQ ID NO: 53) (Skipper et al., 2018), both fragments were cloned into a BspTI/Kpn2I digested pMDLg/p-PCS-hyPBase (SEQ ID NO: 53).

To construct pU6-Chimeric_BB-CBh-eGFP (SEQ ID NO: 56), the eGFP gene was amplified from pCCL/PGK-eGFP (SEQ ID NO: 49) using MVA66+MVA67 (SEQ ID NOS: 28-29) and inserted into BshTI/KpnI digested pX330-U6-Chimeric_BB-CBh-hSpCas9 (Addgene plasmid #42230).

To construct pU6-SpCas9_sgRNA2.0-CBh-eGFP (SEQ ID NO: 57), the sgRNA cassette was amplified from pU6-Chimeric_BB-CBh-eGFP (SEQ ID NO: 56) with MVA111+MVA114 (SEQ ID NOs: 30+32) and MVA113+MVA115 (SEQ ID NOS: 31+33). Both fragments were cloned into pU6-Chimeric_BB-CBh-eGFP (SEQ ID NO: 56).

pU6-SpCas9_sgRNA2.1-CBh-eGFP (SEQ ID NO: 58), was constructed similarly, but by amplifying the sgRNA cassette from pU6-SpCas9_sgRNA2.0-CBh-eGFP (SEQ ID NO: 57), using MVA111+MVA141 (SEQ ID NOs: 30+34) and MVA113+142 (SEQ ID NOS: 31+35) and cloning the fragments into pU6-SpCas9_sgRNA2.0-CBh-eGFP (SEQ ID NO: 57).

All above clonings were performed using NEBuilder® HiFi DNA Assembly Master Mix (New England BioLabs, Ipswich, MA, USA).

Cloning of sgRNAs into different backbones was performed as previously described (Neldeborg et al., 2019), for insertions into pLentiGuide-puro (SEQ ID NO: 51), digestion was performed using Esp3I, and for insertion into pX330-U6-Chimeric_BB-CBh-hSpCas9 (Addgene plasmid #42230) and therefrom derived backbones BpiI was used. All restriction enzymes were purchased from Thermo Fischer Scientific, Waltham, MA, USA.

Primers used for plasmid construction are listed in table S1 (SEQ ID NOS: 24-35).

### Production of Lentivirus-Derived Particles (LVNPs) and Integrase-Defective Lentiviral Vectors (IDLVs)

Integrase-defective lentiviral vectors and particles devoid of vector genome were produced by standard calcium phosphate transfection of lentiviral packaging plasmids (IDLV: pMDLg/p-RRE-D64V (Cai et al, 2016), pRSV-REV (Addgene plasmid #12253), pMD2.G (Addgene plasmid #12259) plus transfer vector, e.g. pCCL/PGK-eGFP (SEQ ID NO: 49); LVNP: pMDLg/p-RRE-D64V (Cai et al, 2016) plus packaging construct harboring fusion, e.g. pSpCas9-PH-gagpol-D64V (SEQ ID NO: 54), pRSV-REV (Addgene plasmid #12253), pMD2.G (Addgene plasmid #12259)). 3-4×10⁶ HEK293T or Lenti-X (Takara Bio Inc, Shiga, Japan) cells seeded the day before 10 cm dishes were transfected with 3 μg pRSV-REV, 3.75 μg pMD2.G, 13 μg GagPol-encoding plasmid(s) and 13 μg plasmids encoding lentiviral transfers or sgRNAs. The day after transfections, medium was replenished. The virus containing supernatant was harvested the following day by filtration through a 0.45-μm (Sarstedt, Nümbrecht, Germany) and concentrated by ultracentrifugation through a 4 ml, 20% sucrose in PBS cushion, at 25,000 RPM at 4° C. for 2 hours in a Beckman SW28 or SW27 rotor. The medium was refreshed for a second harvest the following day. Virus pellets were resuspended in PBS^{-/-} and stored at −80° C.

Concentrations of HIV-1 p24 was measured by ELISA (XpressBio, Frederick, MD) according to the manufacturers protocol.

For production of IDLVs with no SpCas9 fusions, only pMDLg/p-RRE-D64V (Cai et al., 2016) was used, and for production of LVNPs harboring a C-terminal fusion only pMDLg/p-RRE-D64V-PCS-SpCas9 (SEQ ID NO: 52) was used. For production of N-terminal fusions, pSpCas9-PH-gagpol-D64V (SEQ ID NO: 54) and pMDLg/p-RRE-D64V (Cai et al., 2016) were used at a w/w ratio of 2:1, unless otherwise indicated.

sgRNA1.0, sgRNA2.0 and sgRNA2.1 comprising SEQ ID NOs: 9-23 were used in the examples.

### Vector Titration

Assays for functional titer estimation of different viral vector preparations were conducted by limiting dilution using an EGFP encoding lentiviral vector. 10⁵ HEK293T cells seeded the day before in 6-well plates were transduced with serial dilutions of lentiviral vectors. Three days after transduction, cells were trypsinized and analyzed for EGFP expression by flow cytometry on a NovoCyte Flow Cytometer (ACEA Biosciences, San Diego, CA, USA). Dilutions yielding 1-20% EGFP positive cells were used to calculate the titer using the formula:

TU/ml=(cell count on day of transduction×fraction of positive cells)/(volume of virus)

Western Blot Analysis of SpCas9 Incorporation into IDLVs

Ultracentrifuged lentiviral vectors produced with or without SpCas9 fusions and 2 μM saquinavir were denatured in XT Sample Buffer supplemented with XT Reducing Agent (Bio-Rad, Hercules, CA, USA), separated by SDS-PAGE, and blotted onto a polyvinylidene fluoride membrane. The membranes were blocked with 5% skimmed milk dissolved in TBS/0.05% Tween-20 for an hour followed by an overnight incubation with a FLAG antibody (Sigma-Aldrich). The blots were then washed and incubated with anti-mouse secondary antibodies (Dako, Glostrup, Denmark) and visualized by chemiluminescence using Clarity Western ECL Substrate (Bio-Rad). The antibodies were washed with stripping buffer (Thermo Fisher Scientific), and the membrane was incubated overnight with p24 antibodies (R&D Systems, Minneapolis, MN, USA) followed by anti-mouse secondary antibodies.

### Transduction Procedures

For transduction experiments conducted in 6-well plates, unless otherwise noted, LVNPs or IDLVs corresponding to 300 ng p24 were used. For experiments conducted in 48-well plates, virus particles corresponding to 30 ng p24 were generally used. Polybrene was used at a final concentration of 8 μg/ml. Unless otherwise stated, cells were harvested after 4 days in 6-well plates or 3 days in 48-well plates. Genomic DNA was extracted from cells in 6-well plates by saturated NaCl and precipitated with absolute ethanol. In 48-well plates, cells were washed with PBS and lysed using 150 μl lysis buffer (10 mM TRIS-HCL, PH 7.5; 0.05% SDS, 20 μg/ml Proteinase K (Thermo Fisher Scientific) directly in the wells for 1-2 hours at 37° C., followed by inactivation at 80° C. for 30-60 minutes.

### Indel Detection

150 ng precipitated genomic DNA or 1-5 μl of genomic DNA extract was amplified using Phusion Master Mix (Thermo Fisher Scientific), using 0.5 UM of each forward and reverse primers in a total reaction volume of 50 μl. PCR products were purified by gel extraction (Omega Bio-tek, Norcross, GA, USA), and sequenced by GATC/Eurofins. Indel rates were quantified using ICE (Hsiau et al., 2019). Primers used for amplification and sequencing of target loci are listed in table S2 (SEQ ID NOs: 41-48+64-65).

RNA Isolation from LVNPs and ddPCR Analysis

Total RNA from ultracentrifuged LVNPs was extracted using Roche High Pure miRNA Isolation Kit (Roche Applied Science, Mannheim, Germany), and subjected to DNase I treatment (Thermo Fischer) as prescribed by manufacturer. Yield and purity were evaluated on a DeNovix DS-11 Spectrophotometer. Up to 100 ng DNase treated RNA per 10 μl reaction was used for cDNA synthesis using Maxima H Minus cDNA Synthesis Master mix (Thermo Fischer). cDNA was diluted 256 or 512 times depending on RNA input and quantitative droplet digital PCR (ddPCR) was performed on a QX200™ Droplet Digital™ PCR System with ddPCR Supermix for Probes (No dUTP) (BioRad), using the primers 5′-CCTTCAGCTCAGTGACAGTGG-3′ (SEQ ID NO: 59), 5′-CCGACTCGGTGCCACTTT-3′ (SEQ ID NO: 60) and the probe 5′-FAM-AAATAAGGCTAGTCCGTTATCAACTT-BHQ-1-3′ (SEQ ID NO: 61), and the reaction was prepared according to the manufacturer's protocol.

### Example 2-Incorporation of SpCas9 into Lentivirus-Derived Particles

### Aim

It is the aim to study the ability of SpCas9 to be packaged into lentivirus-derived particles.

### Material & Methods

See Example 1

### Results

Lentiviral particles assemble through multimerization of Gag and GagPol polyproteins at the plasma membrane, causing this assembly of polyproteins enclosed by a segment of the membrane to bud off from the virus-producing cell. Released virus particles are immature and undergo maturation triggered by cleavage of the polyproteins by the viral protease. By pseudotyping lentivirions with a heterologous fusogenic envelope protein, the particles can transduce cells as determined by the specificity of the envelope protein and release their cargo into the cell cytoplasm by direct fusion or through endosomal uptake and escape.

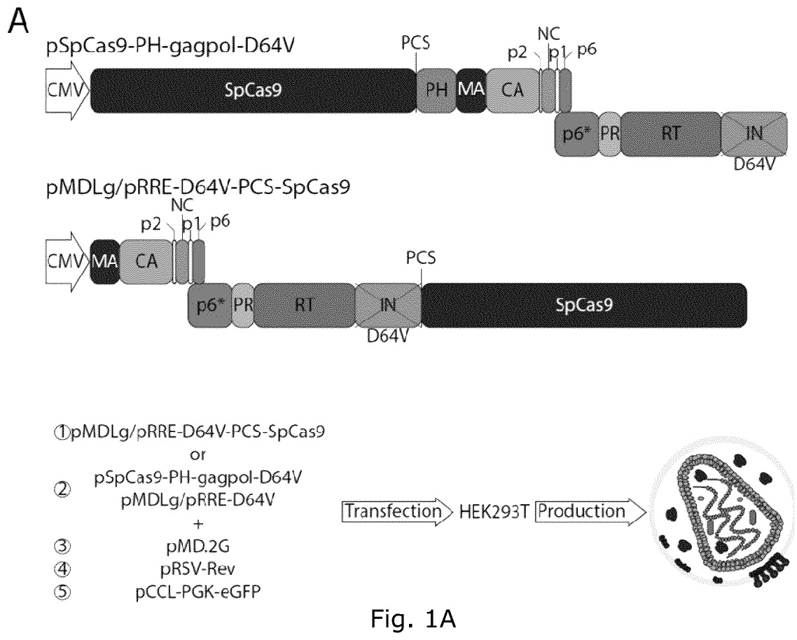

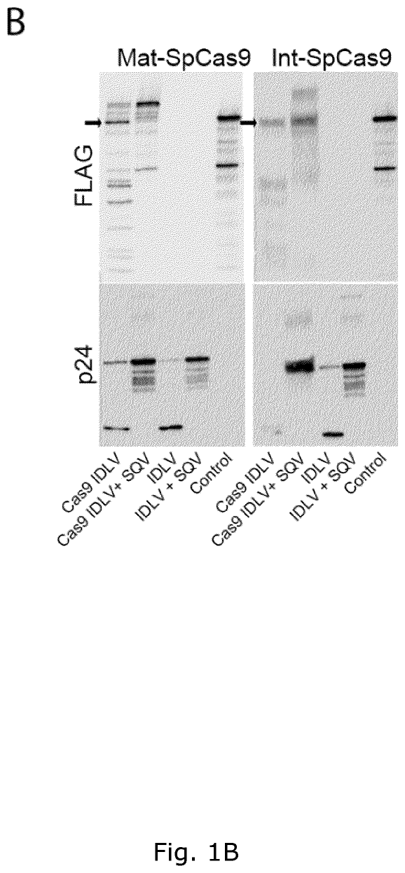

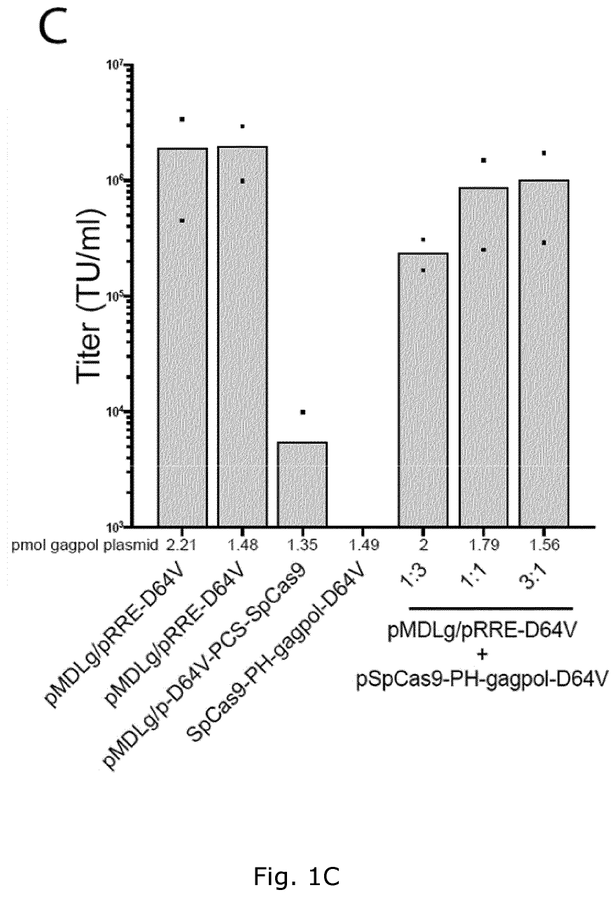

To embed SpCas9 into lentiviral particles, FLAG-tagged SpCas9 was fused either N-terminally to Gag harboring an intervening phospholipase C-δ1 pleckstrin homology (PH) domain, which serves as the membrane targeting motif, or C-terminally to GagPol (FIG. 1A), as previously described (Choi et al., 2016). We refer to these two strategies as Mat-SpCas9 and Int-SpCas9 reflecting that SpCas9 is placed upstream to the matrix protein in the N-terminal fusion and downstream from the integrase protein in the C-terminal fusion, respectively. In both cases, the packaging construct harbored an integrase carrying the D64V mutation rendering the integrase protein catalytically inactive.

First, we tested the protein packaging capabilities of the Mat-SpCas9 and Int-SpCas9 packaging constructs by producing integrase-defective lentiviral vectors (IDLVs) carrying vector RNA with an eGFP expression cassette driven by the phosphoglycerate kinase (PGK) promoter. The protein content of these particles was analyzed by Western blot (FIG. 1B).

For both fusion strategies, we observed a ˜160 kDa band indicative of the presence of SpCas9 in the virus particles. This was confirmed by including cell lysate from HEK293T cells transfected with SpCas9 encoding plasmid as positive control, suggesting that SpCas9 was correctly released from the Gag and GagPol during particle maturation.

In contrast, production of IDLVs in the presence of the protease inhibitor saquinavir (SQV) inhibited the release of p24 and SpCas9 from the Gag/GagPol polypeptides, demonstrating that release of free SpCas9 was achieved by HIV-1 protease-mediated maturation of the viral particles. In virus particles as well as in cell lysates from transfected cells, we observed shorter proteins that were detected with the FLAG antibody. This suggests that shorter SpCas9 derivatives are produced even after standard plasmid DNA transfection potentially due to degradation, but also that shorter variants are present in virus particles potentially due to degradation or unspecific proteolytic cleavage by the viral protease during particle maturation.

As Cas9-mediated genome editing with Cas9 also requires delivery of sgRNAs, which could possibly be delivered using vector RNA carrying a sgRNA expression cassette, we tested the capability of eGFP-encoding IDLVs carrying the fusion polypeptides to deliver the transgene-encoding vector RNA (FIG. 1C). As we have previously observed with virus particles carrying the N-terminal fusion Gag/GagPol variants (Cai et al., 2014b, Skipper et al., 2018) a functional titer indicative of successful eGFP gene transfer could not be registered with the Mat-SpCas9 format (production using pSpCas9-PH-gagpol-D64V only), whereas transductional titers of ILDVs carrying normal Gag/GagPol were 2×10⁶ TU/ml. The titer of Int-SpCas9 IDLVs carrying SpCas9 fused to the D64V integrase at the C-terminus of GagPol (production using pMDLg/p-D64V-PCS-SpCas9) was reduced to below 10⁴ TU/ml. These findings confirm that virus particles built of Mat-SpCas9 fusion Gag/GagPol variants only are incapable of transferring and reverse-transcribing vector RNA.

Notably, by titrating packaging plasmid DNA encoding normal Gag/GagPol (but still containing the D64V mutation) into the plasmid DNA mix used for transfection as part of the virus production, functional eGFP gene transfer was restored (FIG. 1C), allowing titers to reach 10⁶ TU/ml using pSpCas9-PH-gagpol-D64V and pMDLg/pRRE-D64V packaging plasmids in a 1:3 ratio. Taking into account that the constructs used for production of IDLVs carrying the SpCas9 fusion were larger than pMDLg/pRRE-D64V, this titer reduction could possibly reflect a reduction in moles of Gag/GagPol-encoding plasmid used for the production. However, the titer was gradually reduced by increasing the amount of SpCas9-containing Gag/GagPol in the Mat-SpCas9 particles (FIG. 1C), demonstrating a negative impact of SpCas9 protein on the gene transfer rate.

These data demonstrate the capacity of lentivirus-derived particles to transport and deliver RNP complexes consisting of SpCas9 and scaffold-modified sgRNAs in a DNA-free fashion that does not involve transport of genetic material except for the sgRNA itself. Using two previously reported strategies for delivery of zinc-finger nucleases (Cai et al., 2014a; Cai et al., 2016) and piggyBac DNA transposases (Cai et al., 2014b; Skipper et al., 2018) in lentiviral particles, based on fusing SpCas9 to either the N-terminus of Gag (Mat-SpCas9) or the C-terminus of GagPol (Int-SpCas9), we studied the capacity of virions to incorporate both SpCas9 and sgRNA. As previously reported (Choi et al., 2016), production of LVNPs loaded with foreign protein has negative impact on the functionality of the virus particle and generally results in reduced virus yields. However, by titrating in wildtype Gag/GagPol during virus production, allowing chimeric virus particles consisting of both wildtype and fusion Gag/GagPol to be generated, yields of functional particles can be partially recovered, allowing larger production of LVNPs.

### Conclusion

The data demonstrates that both Mat-SpCas9 and Int-SpCas9 can be used for incorporating SpCas9 into the virus particles.

The data furthermore concludes that Mat-SpCas9 fused to Gag/GagPol is not able to transfer and reverse-transcribe vector RNA. Thus, sgRNA will not be transcribed if using vector RNA carrying a sgRNA expression cassette.

The data also concludes that incorporating SpCas9 into LVNPs, particles containing SpCas9 fused to the N-terminus of Gag/GagPol resulted in markedly higher editing efficacies. Notably, wildtype Gag/GagPol was added to these particles to optimize yield and overall performance.

### Example 3—Traceless Delivery of SpCas9 and sgRNAs in Lentiviral Particles

### Aim

To obtain the most optimal way of co-delivering sgRNA with the SpCas9 protein.

### Material & Methods

See Example 1

### Results

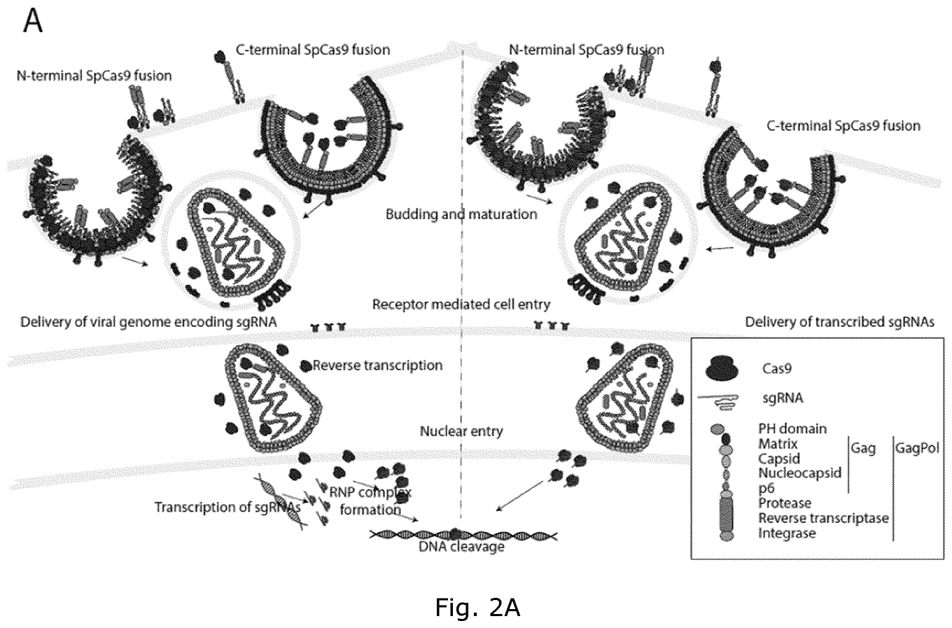

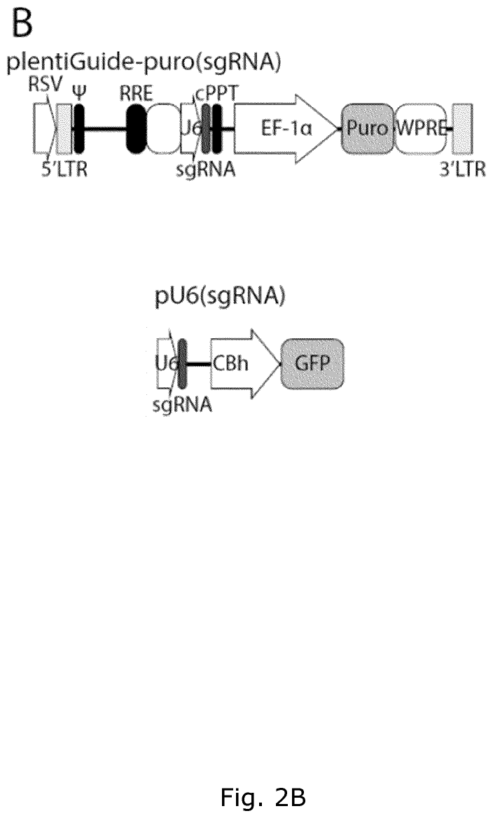

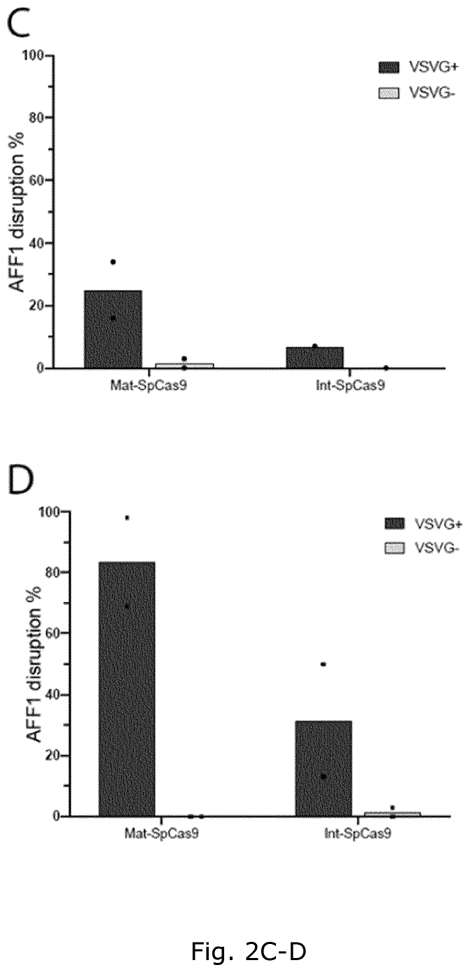

We investigated two routes for sgRNA delivery (shown schematically in FIG. 2A) based on (i) packaging of a sgRNA-encoding transfer vector in SpCas9-loaded IDLVs allowing production of sgRNAs in the transduced cells (FIG. 2A, left side) and (ii) overexpression of sgRNAs from transfected plasmid DNA in virus-producing cells allowing functional genome editing only if sgRNAs are successfully incorporated into SpCas9-loaded virus particles (FIG. 2A, right side).

Using both the Mat-SpCas9 and Int-SpCas9 formats, we first produced SpCas9-loaded IDLVs containing a lentiviral vector with an U6-driven expression cassette encoding a sgRNA targeting the AFF1 gene (FIG. 2B). We have previously seen this particular sgRNA to induce efficient SpCas9-directed DNA cleavage in the human AFF1 locus. For both formats, targeted DNA cleavage was identified in the AFF1 locus in transduced HEK293T cells, resulting in indel rates of 25% and 8% in cells treated with Mat-SpCas9- and Int-SpCas9-containing IDLVs, respectively (FIG. 2C).

In contrast, IDLVs devoid of the VSV-G surface protein did not produce indels in AFF1. Together, these data document activity of SpCas9 protein after VSV-G-directed uptake of lentiviral particles in transduced cells.

Although these data also suggest that sgRNA encoded by co-delivered lentiviral vectors support RNA-guided DNA cleavage, it could not be excluded from these data that SpCas9 activity was guided from sgRNAs produced from the lentiviral vector plasmid and packaged with SpCas9 protein in the lentivirus particles.

To investigate this possibility, we produced AFF1-directed sgRNAs in virus-producing cells by including a standard sgRNA expression plasmid, which did not encode packageable vector RNA, in the cocktail for plasmid DNA transfection of producer cells. Interestingly, SpCas9-loaded LVNPs produced under these conditions induced increased levels of indel formation in transduced HEK293T cells, leading to indel rates of 80% and 30% with Mat-SpCas9 and Int-SpCas9 formats, respectively (FIG. 2D). Again, only background indel formation was observed with VSV-G-negative virus particles, demonstrating that SpCas9 activity relied on VSV-G-dependent virus uptake. Notably, the Mat-SpCas9 resulted in the highest level of indel formation, suggesting that this configuration was more potent, most likely due to the expected larger content of SpCas9 in Mat-SpCas9 particles relative to Int-SpCas9 particles, which receive SpCas9 only as part of GagPol.

### Conclusion

These data conclude that sgRNAs expressed in producer cells are incorporated in lentivirus particles and are capable of directing co-delivered SpCas9 protein to a predetermined target locus in recipient cells. Hence, SpCas9/sgRNA-loaded LVNPs result in transient, ‘traceless’ delivery of CRISPR tool kits leading to high levels of editing in transduced cells.

### Example 4—Improved Indel Formation by Incorporation of Scaffold-Modified sgRNAs in SpCas9-Loaded LVNPs

### Aim

To further optimize targeted DNA cleavage using LVNP-directed delivery by packaging scaffold-modified sgRNA together with SpCas9 in LVNPs.

### Material & Methods

See Example 1

### Results

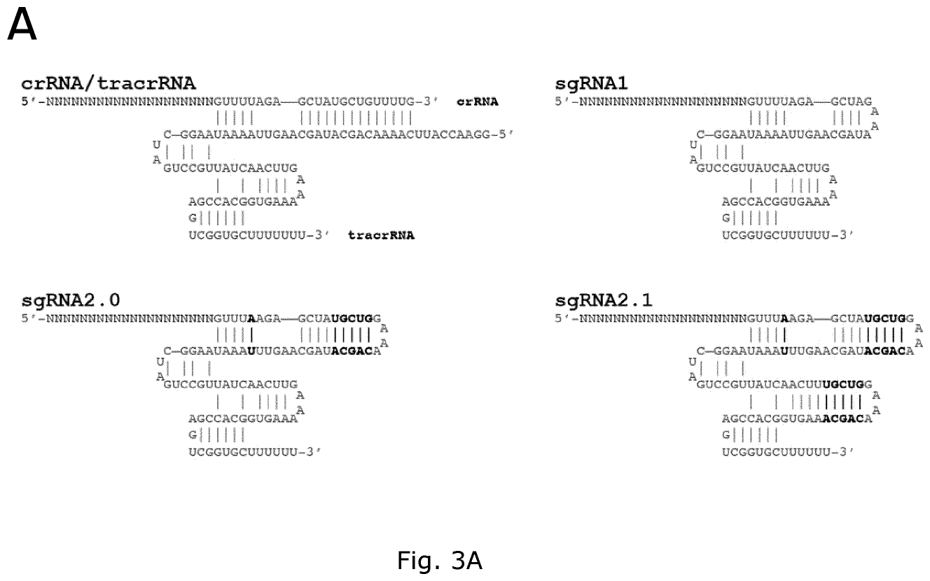

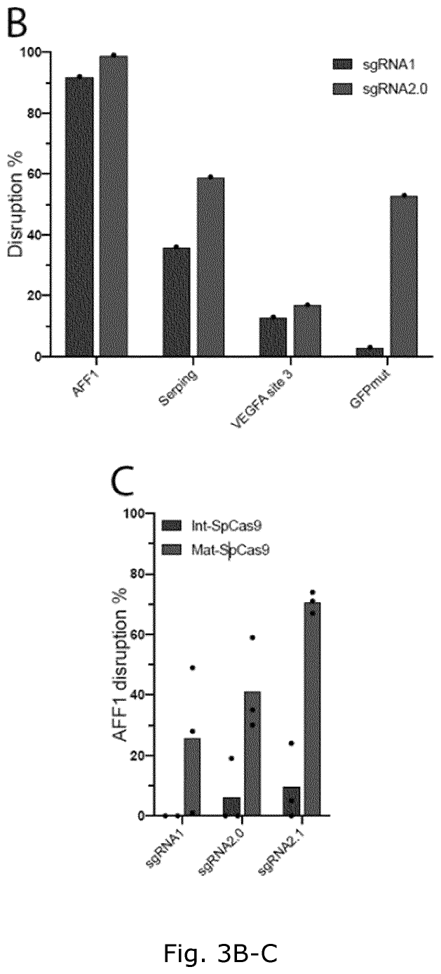

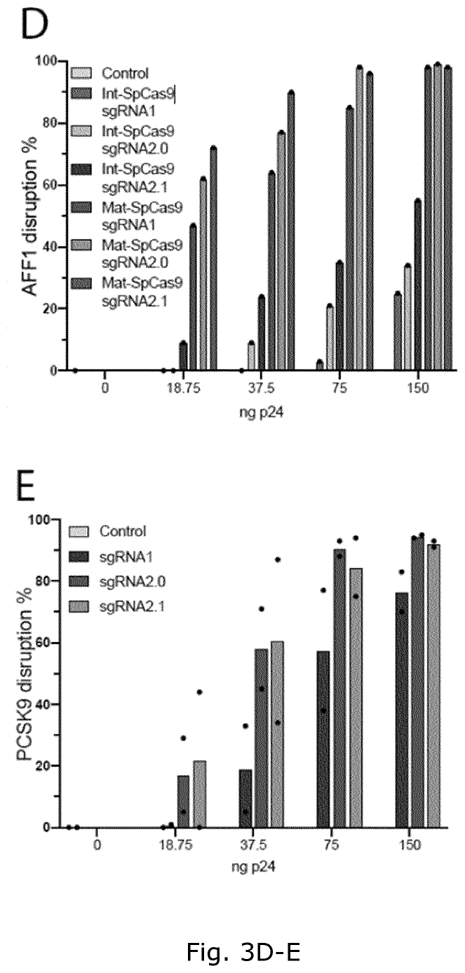

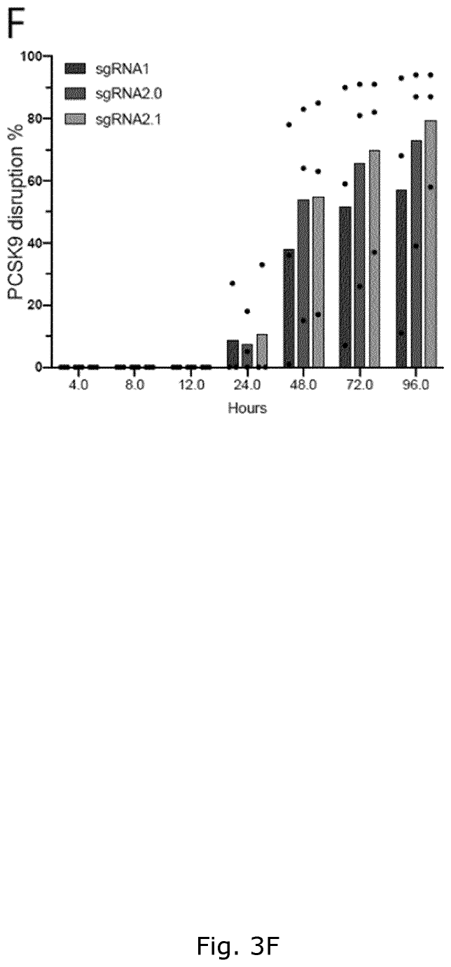

Schematics of the wild-type SpCas9 crRNA/tracrRNA (SEQ ID NOs: 4-5) and three different sgRNA versions (SEQ ID NOs: 6-8) are shown in FIG. 3A. Initially, we compared the original sgRNA (sgRNA1) and an improved version (sgRNA2.0) (FIG. 3A). Using the Mat-SpCas9 format, we produced a series of SpCas9-loaded LVNPs, packaged with either sgRNA1 or sgRNA2.0, targeting a predetermined site in three different human loci (AFF1, SERPING1, VEGFA (site 3)) or a site in a mutated version of the eGFP gene (eGFPmut). We then transferred SpCas9/sgRNA-loaded LVNPs to HEK293T cells or to HEK293 cells harbouring a stably integrated copy of the eGFPmut reporter gene cells. Using the standard sgRNA1 configuration, variable levels of indel formation was achieved in the four different loci ranging from 3% in the GFPmut locus to 90% in AFF1 (FIG. 3B).

However, we found that the indel formation was increased at all four loci by treating the cells with LVNPs carrying sgRNA of the sgRNA2.0 configuration. For GFPmut-targeted cleavage, in particular, the improvement was dramatic, leading to an indel rate above 50%. For SERPING1-directed DNA cleavage, indel rates increased from 37% to near 60%. Even targeted disruption of AFF1 could be further optimized using the sgRNA2.0 scaffold, resulting in cleavage of almost all targeted alleles (98%) in the cell population.

Encouraged by this observation, we extended the analysis to include another sgRNA scaffold, sgRNA2.1 (FIG. 3A). First, we produced Mat-SpCas9 and Int-SpCas9 LVNPs containing sgRNA1, sgRNA2.0 or sgRNA2.1 targeting the AFF1 locus and transduced HEK293T cells in a 48-well format using a fixed LVNP dose (15 ng p24).

Under these conditions, AFF1 gene disruption was reduced to 26% in cells treated with sgRNA1-containing LVNPs, whereas LVNPs carrying sgRNA2.0 and sgRNA2.1 performed markedly better leading to gene disruption rates of 41% and 71%, respectively (FIG. 3C).

In our standard 6-well format, we then exposed HEK293T cells to different LVNP dosages ranging from 0 to 150 ng p24. Overall, we again found markedly higher levels of AFF1 gene disruption with Mat-SpCas9 format LVNPs than with Int-SpCas9 LVNPs (FIG. 3D). However, for both formats, a clear correlation between LVNP dose and indel formation in transduced cells was observed. Notably, at almost all LVNP dosages, the highest level of targeted indel formation was achieved with LVNPs carrying sgRNA2.1. Only for highest Mat-SpCas9 LVNP dosages (75 and 150 ng p24), for which indel formation approached 100%, an effect of the improved sgRNA scaffold could not be detected (FIG. 3D). By including scaffold-modified sgRNAs in LVNP, it was possible to achieve high disruption rates even at low LVNP dosages. Most remarkably, disruption rates of 70% was evident in cells treated with 16-fold lower doses of LVNPs relative to our standard setup using a LVNP dose corresponding to 300 ng p24.

Finally, we produced Mat-SpCas9 LVNPs with sgRNA1, sgRNA2.0 or sgRNA2.1 targeting another locus, the Pcsk9 gene, in mouse cells (FIG. 3E). In this case, we also observed a correlation between LVNP dose and indel formation. However, for targeting of Pcsk9 in murine AML12 cells, LVNPs loaded with sgRNA2.0 and sgRNA2.1 performed quite similarly with markedly higher gene disruption capacity than sgRNA1-loaded LVNPs (FIG. 3E).

To understand the kinetics of LVNP-directed SpCas9/sgRNA delivery in relation to indel formation, we analyzed the emergence of targeted indels in the Pcsk9 locus in AML12 cells over the course of 96 hours (FIG. 3F). Cells were reverse transduced and lysed at different time intervals after the initial exposure to LVNPs allowing the progression of indel formation to be assessed. Notably, up to 12 hours after exposure of the cells to LVNPs, targeted indels could not be detected with any of the three sgRNA scaffolds (sgRNA1, sgRNA2.0, and sgRNA2.1). After 24 hours, indels appeared in cells exposed to LVNP containing sgRNA1, sgRNA2.0 and sgRNA2.1, and over the next three days, indel rates generally increased, reaching a peak level for LVNPs loaded with sgRNA2.1 at 80% (FIG. 3F).

### Conclusion

In conclusion, the data demonstrate that gene disruption activities based on LVNP-directed co-delivery of SpCas9 and sgRNA is robust. This activity can be optimized by the use of scaffold-modified sgRNAs allowing fast targeted DNA cleavage even with small LVNP dosages.

One might speculate that increased affinity between Cas9 and the sgRNA may result in a more stable RNP complex in recipient cells facilitating an increased proportion of Cas9 reaching the nucleus complexed with the sgRNA. Altogether, our findings support the use of scaffold-optimized sgRNAs for achieving higher levels of targeted DNA cleavage using the LVNP technology.

### Example 5—Quantification of sgRNA

### Aim

To quantify the sgRNA content in SpCas9-containing lentivirus particles.

### Material & Methods

See Example 1

### Results

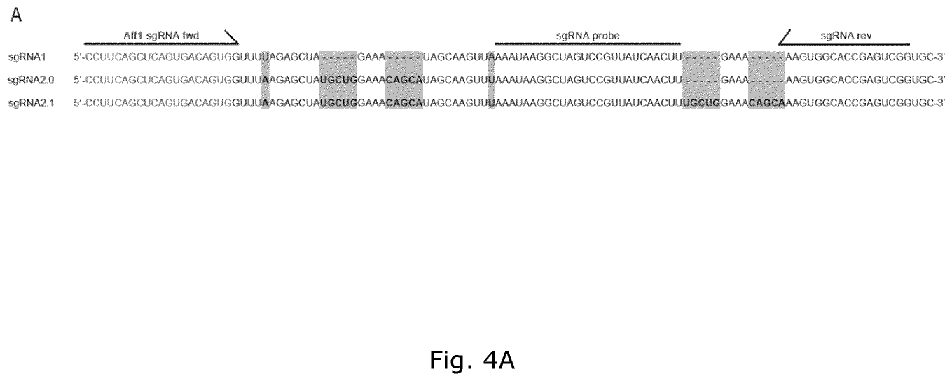

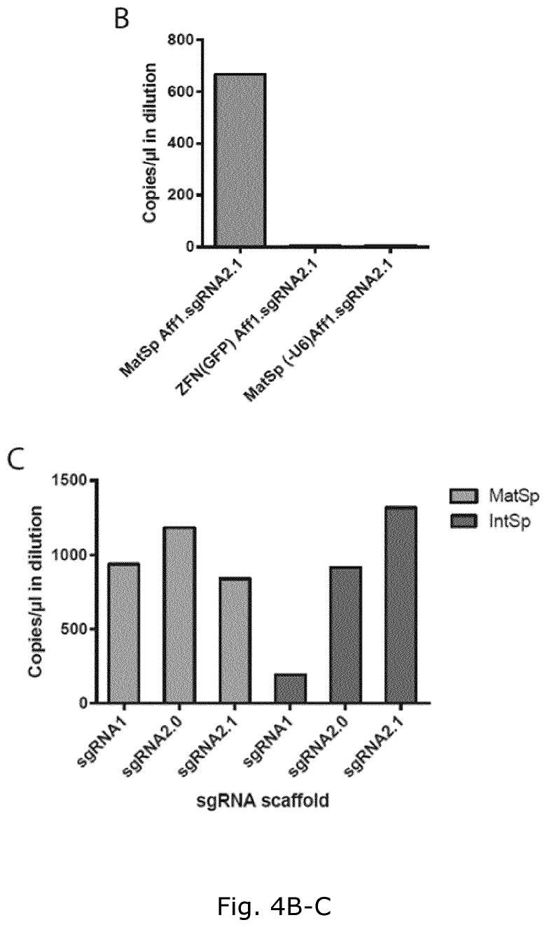

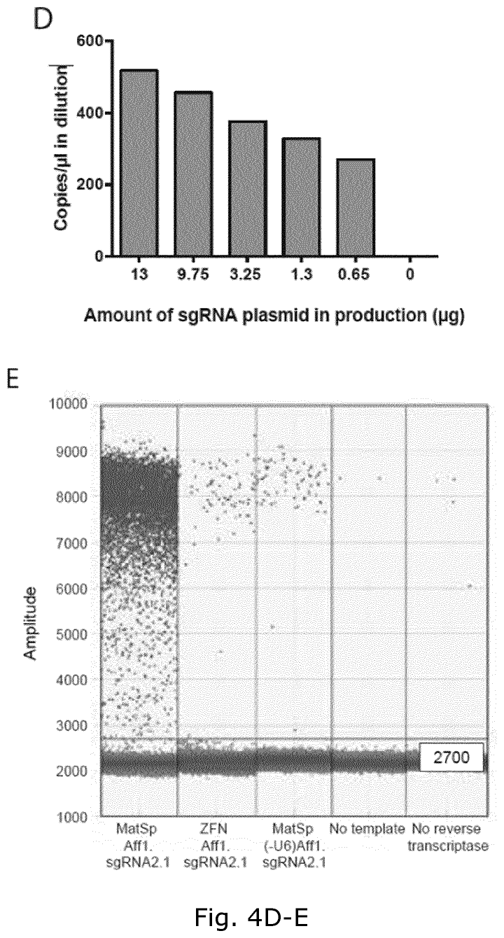

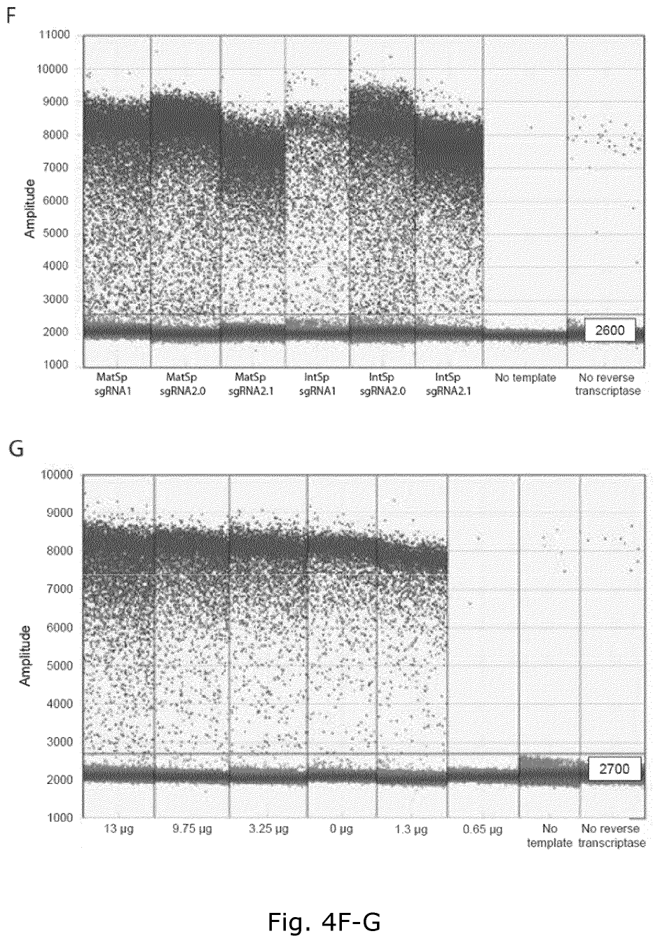

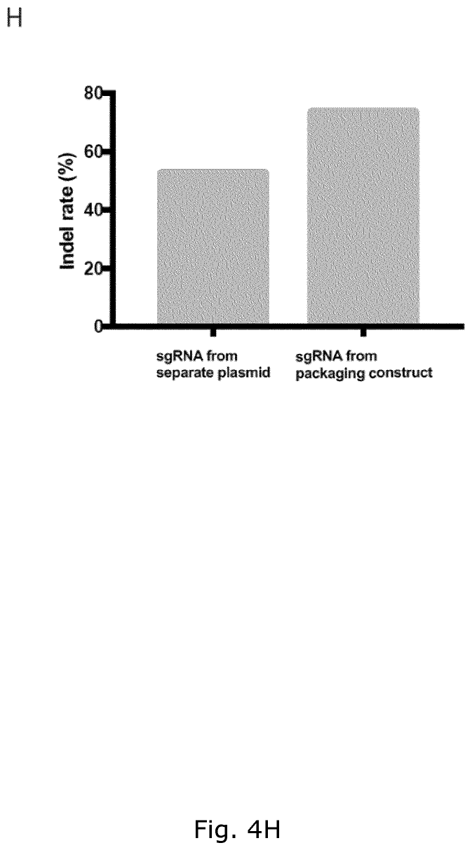

To determine the amount of transcribed sgRNA packaged in the LVNPs, we designed a TaqMan-based primer/probe set for use in droplet digital PCR (ddPCR). FIG. 4A shows a schematic drawing of the three sgRNA backbones (except for 3′ uracils) with a targeting region towards AFF1, with the differences marked in grey (SEQ ID NOs: 9-11). The probe and reverse primer were designed to be universal for all three sgRNA scaffolds (SEQ ID NOs: 60-61), whereas the forward primer was spacer-specific targeting in this case AFF1 sgRNA (FIG. 4A). The area for hybridising of the primers and probes on the sgRNAs are indicated above the sequences of the sgRNA scaffolds and relates to SEQ ID NOs: 66-68. Using sgRNA-specific ddPCR, to our knowledge for the first time on virus-embedded sgRNAs, we first addressed whether incorporation of sgRNA in LVNP was dependent on co-packaging of SpCas9 protein.

Using the sgRNA2.1 scaffold, we compared the levels of sgRNA in SpCas9-loaded LVNPs with LVNPs carrying ZFNs targeting eGFP fused to the N-terminus of Gag/GagPol (SEQ ID NO: 55), for which we have previously demonstrated effective packaging in lentiviral particles (Cai et al., 2014a). For LVNPs carrying the SpCas9 fusion, we detected high levels of sgRNA in the virus particles corresponding to 667 sgRNA copies per μl in MatSpCas9 format LVNPs (FIGS. 4B and 4E).

For LVNPs carrying ZFNs, only background levels of sgRNA could be detected in the particles (6 copies per μl). To control for potential background signals from plasmid DNA, we included Mat-SpCas9 LVNPs produced in cells co-transfected with plasmid DNA encoding sgRNA2.1, but lacking the U6 promoter (thus limiting sgRNA production), as an additional negative control (MatSp (-U6)Aff1.sgRNA2.1).

SgRNA amplicons were detected neither by analysis of these LVNPs nor by running PCRs on reverse transcriptase-negative samples prepared from sgRNA-containing LVNPs, suggesting that the analysis was specific for LVNP-incorporated sgRNAs.

Interestingly, the level of AFF1-targeting sgRNA was roughly similar for MatSpCas9 LVNPs carrying the three different sgRNA scaffolds, whereas higher copy numbers of sgRNA2.0 and sgRNA2.1 species relative to sgRNA1 were measured in Int-SpCas9 LVNPs (FIGS. 4C and 4F).

These data demonstrate that the relative level of incorporation of sgRNA1, sgRNA2.0, and sgRNA2.1 was dependent on the LVNP format. For Mat-SpCas9 LVNPs, the three types of sgRNAs were packaged equally effectively in the virus particles, whereas a clear difference in sgRNA packaging rates was observed in Int-SpCas9 format LVNPs. Here, optimized scaffolds resulted in more potent packaging of sgRNA2.0 and sgRNA2.1. Notably, for both Mat-SpCas9 and Int-SpCas9 formats, we observed increased DNA cleavage activity of LVNPs loaded with scaffold-optimized sgRNAs at different genomic loci and in different cell lines.

### Conclusion

These data concludes that the packaging of sgRNAs in the LVNP is dependent on SpCas9, supporting the notion that SpCas9 and sgRNA are incorporated into lentiviral particles as a reassembled ribonucleoprotein (RNP) complex consisting of sgRNA associated with Gag/GagPol-fused SpCas9.

The results also demonstrate that incorporation of sgRNAs in Mat-SpCas9 LVNPs was saturated due to prominent SpCas9 incorporation in these particles, whereas an effect of improved scaffolds could be detected only in Int-SpCas9 LVNPs packaged with fewer SpCas9 proteins.

### Example 6—Saturation of SpCas9

### Aim

To investigate potential saturation of SpCas9 with sgRNA.

### Material & Methods

See Example 1

### Results

To investigate this, we produced a series of LVNPs using varying amounts of sgRNA-encoding plasmid in the production and measured levels of LVNP sgRNA by ddPCR (FIGS. 4D and 4G). Notably, the highest level of sgRNA was observed with the highest amount of sgRNA-encoding plasmid, and LVNP sgRNA levels gradually decreased with reduced amount of plasmid DNA.

However, even with a very low amount of sgRNA-encoding plasmid in the production (0.65 μg), we still observed more than 50% of the sgRNA level obtained with our standard amount of plasmid (270.89 copies/μl and 517.93 copies/μl with 0.65 μg and 13 μg plasmid, respectively).

### Conclusion

These data show that the sgRNA amount in LVNPs depends on the amount of transcribed sgRNA in the producer cells, but also that high levels of sgRNA are packaged in LVNPs even with low production of sgRNAs, supporting the notion that sgRNAs are actively recruited into virus particles by SpCas9.

### Example 7—Optimising Expression Method of sgRNA

### Aim

To investigate potential expression of sgRNA together with SpCas9.

### Material & Methods

See Example 1

### Results

Based on these findings, we reasoned that production of SpCas9/sgRNA-loaded LVNP could be further optimized by adding the sgRNA expression cassette to the SpCas9-GagPol-encoding packaging construct. Therefore, we produced SpCas9-loaded LVNPs in the presence of a separate sgRNA-encoding plasmid and using a packaging construct harboring an additional sgRNA-encoding cassette positioned upstream of the CMV promoter in the reverse orientation, and measured the indel rate in HEK293T cells exposed to these viral preparations (FIG. 4H). Notably, a side-by-side comparison of these LVNPs resulted in higher levels of indel formation by including the sgRNA expression cassette on the packaging construct.

### Conclusion

These data demonstrate that the performance of SpCas9-loaded LVNPs can be further optimized by modifying the LVNP production process.

### Example 8: Materials and Methods

LVNP1.0, as used in the examples 8-15, describes LVNPs comprising Int-SpCas9. LVNP2.0, as used in the examples 8-15, describes LVNPs comprising Mat-SpCas9. LVNP2.1, as used in the examples 8-15, describes LVNP2.0, produced with a 70/30 ratio between the packaging plasmids pGagPol-D64V and pSpCas9-PH-gagpol-D64V (Mat-SpCas9) and sgRNA2.1.

LVNP2.2, as used in the examples 8-15, describes LVNP2.1 produced with a 60/40 ratio between the plasmids pCCL-PGK-eGFP or pCCL-PGK-mCherry and pU6-sgRNA-CBh-eGFP.

### Cell Lines

All cells were cultured in DMEM (Sigma-Aldrich) supplemented with 5% FBS, 100 U/ml penicillin, and 100 μg/ml streptomycin (Thermo Fischer Scientific). Transgenic cell lines were maintained in puromycin (1 μg/mL) (Thermo Fisher Scientific) or blasticidin (5 μg/mL) (Thermo Fisher Scientific). Cells were maintained at 60-90% confluence at 37° C. with 5% carbon dioxide and repeatably tested negative for mycoplasma (Eurofins Genomics).

### Plasmid Construction

Plasmids were constructed using NEBuilder® HiFi DNA Assembly Master Mix (New England BioLabs) and deposited to Addgene.

### PGK-FahMut-P2A-eGFP-IRES-Puro

A third-generation LV vector encoding PGK-FahMut-P2A-eGFP-IRES-Puro (FahMut reporter; SEQ ID NO: 109) was designed for KO analysis purposes. The FahMut reporter is composed of Fah CDS1-8mut, intron 8, and CDS9-14 and harbors a splice mutation (G>A) at the last nucleotide of exon 8 leading to aberrant splicing and a premature stop codon in intron 8. Four fragments encoding (i) CDS1-8mut, (ii) intron8, (iii) CDS9-14, and (iv) P2A-eGFP assembled into BamHI/MluI-linearized (Thermo Fisher Scientific) pCCL/PGK-MCS-IRES-Puro (SEQ ID NO: 106) using NEBuilder® HiFi DNA Assembly Master Mix (New England Biolabs). Fragments 1-3 were purchased as gene fragments (TWIST Bioscience) and PCR amplified with JH9 and JH10, JH11 and JH12, and JH13 and JH14, respectively, and fragment 4 was PCR-amplified from LentiCRISPRv2-eGFP (Addgene #82416) using primers JH15 and JH16. Primers are listed in Table S1 (SEQ ID NOs: 86-93)

Primers for indel analysis are listed in Table S2 (SEQ ID NOs: 101-103).

Cloning of pSpCas9-PH-gagpol-D64V (Mat-SpCas9) and pMGLg/pRRE-D64V-PCS-SpCas9 (Int-SpCas9)

See Example 1 for cloning of pSpCas9-PH-gagpol-D64V (Mat-SpCas9) [LVNP2.0] and pMGLg/pRRE-D64V-PCS-SpCas9 (Int-SpCas9) [LVNP1.0].

### Vector for Monitoring of Nuclease Activity

A third-generation LV vector encoding pCCL/PGK-d2eGFP-IRES-puro was constructed by digestion of pCCL/PGK-MCS-IRES-puro (SEQ ID NO: 106) with BamHI (Thermo Fisher Scientific) following insertion of the d2eGFP fragment amplified from pT2/UASTK-d2eGFP-SV40-neo (SEQ ID NO: 107) using primers SA1 and SA2 and assembled by NEBuilder® HiFi DNA Assembly Master Mix (New England Biolabs. Primers are listed in Table S1 (SEQ ID NOs: 84-85).

pCCL-PGK-mCherry (SEQ ID NO: 108) used as transfer vector in experiments with LVNP2.2 targeting d2eGFP.

Spacer sequence for targeting of d2eGFP is listed in Table S2 (SEQ ID NO: 94) sgRNA2.1 targeting d2eGFP comprises SEQ ID NO: 104.

### Generation of Transgenic Cell Lines

For generation of FahMut reporter cell line, 1×10⁵ HEK293T cells were seeded in 6-well plates. To generate cells predominantly harbouring a single transgene cassette cells were transduced in serial dilutions of LV vector preparations to achieve transduction at a low MOI (<0.1). The medium was changed 24 h after transduction, and puromycin selection (1 μg/mL) was applied for 5-7 days. The lowest dose with surviving cells were expanded and kept under puromycin selection during expansion and experiments.

sgRNA2.1 targeting Fah comprises SEQ ID NO: 105.

### LV Vector and LVNP Production

Both LV, IDLV, and LVNP were produced as previously described in Ryo et al. (2019). Ultracentrifugation was performed at 25,000 RPM at 4° C. for 2 hours in a Beckman SW27 or SW28 rotor. Pellets were resuspended in 85 μL PBS overnight (4° C.), pooled (first and second harvest), and centrifuged at 1,200 RPM to precipitate residual debris. The viral concentration was quantified by p24 ELISA (XpressBio) according to the manufacturer's protocol and stored at −80° C. in aliquots until use.

### Titer Determination

The functional titer was estimated by limiting dilution. 100,000 HEK293T were seeded in 6-well plates and transduced with serial dilutions the indicated virus in polybrene (8 μg/mL). After three days, cells were analysed for eGFP expression by flow cytometry on a NovoCyte Flow Cytometer (ACEA Biosciences). Dilutions resulting in 5-20% eGFP positive cells were used to calculate the functional titer:

$$\frac{TU}{mL} = \frac{\begin{matrix}cell count on day of transfection \times \\ fraciton of eGFP positive cells\end{matrix}}{volume of virus}$$

### Flow Cytometry

Cells were washed with PBS, detached by trypsinization, and resuspended in FACS buffer (1% BSA, 2.5 mM EDTA, 25 mM HEPES dissolved in PBS). Cells were analysed on a NovoCyte Flow Cytometer (ACEA Bioscience). Data was analysed in Novo Express or FlowJo.

### Western Blotting

LVNP and IDLV was produced either in the presence or absence of 2 μM saquinavir (SQV). Ultracentrifuged particles (90 ng p24) were lysed in RIPA buffer (Thermo Fisher Scientific) supplemented with 10 mM NaF and 1× complete protease inhibitor cocktail (Roche). The lysate was denaturated in XT Sample Buffer supplemented with XT Reducing Agent (Bio-Rad), separated by SDS-PAGE, and transferred to a polyvinylidene fluoride membrane. The membrane was blocked with 5% skimmed milk dissolved in TBS/0.05% Tween-20 for 1 hour and incubated overnight with a FLAG antibody (Sigma-Aldrich). The membrane was washed and incubated with anti-mouse secondary antibodies (Dako) and visualized by chemiluminescence using Clarity Western ECL Substrate (Bio-Rad). The antibodies were removed with stripping buffer (Thermo Fisher Scientific), and the membrane was incubated overnight with a p24 antibody (R&D Systems) followed by anti-mouse secondary antibodies.

### Transduction Procedures

Cells were plated in 24-well plates (Sarstedt) at a density of 50,000 cells/well (HEK293T, HEK293T-Vegfa, and FahMut reporter) or 30,000 cells/well (AML12) and incubated overnight unless stated otherwise. Transduction was carried out in a total volume of 500 μL fresh medium containing polybrene (8 μg/mL) and the indicated amount virus. Cells were harvested 3 days post-transduction and used for downstream analysis.

### Genomic DNA Extraction and PCR

Genomic DNA was isolated by NaCl/EtOH precipitation as previously described in Skipper et al. (2018). Following resuspension in TE-buffer, 1 μL (1-20 ng) was used for PCR amplification of the target region using Phusion Master Mix (Thermo Fisher Scientific). PCR products were purified by gel extraction (Omega Bio-tek) or PCR clean-up (SAP/EXO). A solution of 0.5 μL FastAP, 0.5 μL Exo1, 9 μL PCR product, and H₂O to a final volume of 18 μL was incubated at 37° C. for 15 min followed by inactivation at 85° C. for 15 min in a thermocycler. The resulting amplicon was sequenced by Eurofins Genomics. The resulting indel frequencies were deconvoluted by ICE analysis as described in Conant, D et al. (2022),

Primer sequences for indel analysis and sgRNA spacer sequences are listed in Table S2 (SEQ ID NOs: 36-48, 64-65, 94-103 & 110-112)

Determination of sgRNA Abundance Using ddPCR

Total RNA from ultracentrifuged LVNPs was extracted using Roche High Pure miRNA Isolation Kit (Roche Applied Science) and treated with DNase I (Thermo Fisher Scientific) to remove any residual plasmid DNA. Total RNA from recipient cells was isolated as previously described in Thomsen et al (2022). Both yield and purity were evaluated on a DeNovix DS-11 Spectrophotometer. Equal amounts of input RNA were used for cDNA synthesis using Maxima H Minus cDNA Synthesis Master mix (Thermo Fisher Scientific). The cDNA was diluted 2 times (recipient cells) or 512 times (LVNPs) and quantitative droplet digital PCR (ddPCR) was performed on a QX200™ Droplet Digital™ PCR System with ddPCR Supermix for Probes (No dUTP) (BioRad) according to the manufacture. A universal probe (SEQ ID NO: 61) and reverse primer (SEQ ID NO: 60) was used, and forward primers SEQ ID NO: 59, SEQ ID NO: 69, SEQ ID NO: 70, and SEQ ID NO: 71, was used for targets AFF1, PCSK9, VEGFA (site 1), and Serping1, respectively.

### Nucleofection

To evaluate on/off-target disruption of the Pcsk9 locus in AML12, 3.2 μg chemically modified (2′-O-Methyl at 3 first and last bases, 3′ phosphorothioate bonds between first 3 and last 2 bases) sgRNA (Synthego) and 6 μg Cas9 protein (Alt-R SpCas9 Nuclease V3, IDT) were incubated at 25° C. for 15 minutes. The RNP solution was mixed with 2×10⁵ AML12 cells in 20 μL OptiMEM. Subsequently, cells were nucleofected using a 4D-nucleofector device (Lonza, Switzerland) in 20-μL Nucleocuvette strips (Lonza) using the program CM-138 set to P3 buffer. Cells were reseeded in 6-well plates (Sarstedt) in DMEM and for downstream analysis. Primers for indel analysis are listed in Table S2 (SEQ ID NOs: 44, 48 & 95-96). Spacer sequence for synthetic sgRNA is listed in Table S2 (SEQ ID NO: 40)

### ChIP-qPCR

ChIP-qPCR against MRE11 was carried out using a scaled down version of the DISCOVER-seq protocol as described in Wienert, B et al. (2020). In brief, 500,000 AML12 cells were seeded (day 0) and transduced (day 1) using 180 ng p24 LVNP2.2 for each time point. Cells were harvested, crosslinked in 1% formaldehyde, washed in PBS, and stored at −80° C. until use. Crosslinked cells were lysed using 1 mL LB1 followed by 1 mL LB2, and lastly 100 μl LB3. Lysed nuclear extract was sonicated for 15 minutes in 30 second pulses on a Bioruptor (Diagenode) and mixed with 185 μl LB3 and 15 μl 20% Triton-X (Sigma-Aldrich). 5 μl lysed nuclear extract was stored as input DNA. The remaining solution was incubated overnight with Dynabeads protein a (Thermo Fisher Scientific) prepared from 10 μl stock bead slurry bound to 1 μg anti-MRE11 (Abcam, ab208020) per sample. Beads were washed 5× in RIPA buffer, 1× in TBS before the crosslinking was reversed in 200 μl elution buffer, while rotating overnight at 65° C. in a hybridization oven alongside the input DNA (5 μL) diluted in 195 μl elution buffer. The eluate was treated with 8 μL 10 mg/mL RNAse A (Thermo Fisher Scientific) for 30 minutes at 37° C. followed by 4 μL of 20 mg/ml Proteinase K (Thermo Fisher Scientific) treatment for 1 hour at 55° C. Hereafter, DNA was purified from the samples using a MiniElute PCR Purification kit (Qiagen) according to the manufacturer and eluted in 33 μl nuclease free water. Purified DNA (4.8 μl) was used as template in a 10 μl qPCR reaction using the RealQ Plus 2× Master Mix Green without ROX (Ampliqon). The qPCR was run in technical triplicates. The enrichment was calculated as: Enrichment=2ΔCt (on-target region)/2ΔCt (control-region) according to the discovery-seq protocol as described in Wienert, B et al. (2020). Primers used targeting Pcsk9 (SEQ ID NO: 72 and SEQ ID NO: 73) and primers for reference target (SEQ ID NO: 74 and SEQ ID NO: 75) was used for amplification.

### In Vivo Experiments

Mice were kept on a 12 h/12 h light/dark cycle at the Animal Facilities at the Department of Biomedicine, Aarhus University, Denmark. Mice had ad libitum access to Altromin maintenance feed (Altromin), and water. Animals were handled in accordance with the “Statement for the Use of Animals in Ophthalmic and Vision Research” from the Association for Research in Vision and Ophthalmology (ARVO).

### Subretinal Injection

8-week-old, male C57Bl/6J mice were purchased from Janvier Labs and allowed to acclimate for a week. Mice (n=10) were anaesthetized by medetomidine hydrochloride 0.5-1 mg/kg (Cepetor) and ketamine 60-100 mg/kg (Ketador). One drop of 1% tropicamide solution (Mydriacy) was used for pupil dilation, and carbomer eye gel (2 mg/g, Viscotears) was used to lubricate the eyes during seduction. The subretinal space was accessed via a transscleral posterior approach using an OPMI 1 FR PRO Surgical microscope (Zeiss), and mice received an unilateral injection with 2 μl (16 ng p24) of LVNP2.2 (encoding a sgRNA targeting Vegfa (site 1) and a transgene encoding eGFP) as previously described in Askou, A. L. et al. (2019). Atipamezole hydrochloride 0.5-1 mg/kg (Antisedan) was used to bring mice out of sedation. Mice were kept warm on a heating pad until mobile, before being transferred back into their cages. Mice received subcutaneous injections of carprofen 5 mg/kg (Norodyl) immediately after subretinal injection and during the next 3 days after via their drinking water (3.33 mg/100 mL).

### Fundoscopy and Optical Coherence Tomography (OCT)

To detect eGFP expression in vivo, and to inspect retinal structures following subretinal injection of LVNP2.2, fluorescence fundoscopy and OCT was carried out 3 days post-injection according to established protocols as described in Holmgaard, A. B. et al. (2021) using a commercial imaging device for rodents, the OCTintegrated with the Micron IV retinal imaging system and the accompanying Reveal software (Phoenix Research Labs).

### RPE/Choroid/Sclera Flat-Mounts

5 days post-injection, mice were sacrificed (n=3), eyes enucleated, and flat-mounts were prepared as previously described in Askou, A. L. et al. (2017). In brief, eyes were cleaned and fixed in fresh 4% paraformaldehyde at RT for 2 h. The cornea, lens, and neuroretina were removed, and 8 incisions from the periphery to the optic nerve enabled flat mounting of the tissue with the RPE cells facing upward on a SuperFrost® Plus glass slide (Menzel-Glaser). Cover glass was mounted using ProLong® Gold antifade reagent (Invitrogen). Flat-mounts were analyzed for eGFP expression by fluorescence microscopy using a Leica DM IRBE (Leica Microsystems). Images were captured with a Leica DFC 360 FX camera and associated software (Leica Application Suite v3).

### Collection of RPE Cells and FACS

RPE cells were collected and pooled from LVNP2.2-injected mice (n=7) and from non-injected eyes (n=3) according to established protocols as described in Alsing, S. et al. (2022). In brief, hyaluronidase was used to detach the neural retina from the RPE layer followed by enzymatic digestion using trypsin combined with shaking of the eyecup to gently detach the RPE cells from the Bruch's membrane. Following the last centrifugation step, RPE cells were resuspended in FACS buffer (1% BSA, 2.5 mM EDTA, 25 mM HEPES dissolved in PBS) and the RPE cell solution was transferred to a 100 μm cell strainer (Thermo Fisher Scientific). Cells were kept on ice and sorted immediately after collection. Fluorescence-activated cell sorting (FACS) was performed using a three-laser FACS Aria III cell sorter (FACS Core Facility, Department of Biomedicine, Aarhus University). The gating strategy was defined in RPE cells isolated from non-injected eyes. Following FACS, the indel frequency was evaluated in both eGFP negative and eGFP positive populations isolated from both non-transduced and LVNP2.2-injected eyes, by doing nested PCR amplification using first primers SEQ ID NO: 76 and SEQ ID NO: 77, and then three different PCRs using primers SEQ ID NO: 78 and SEQ ID NO: 79, SEQ ID NO: 80 and SEQ ID NO: 81, and SEQ ID NO: 82 and SEQ ID NO: 83. Primers for off-target indel analysis are listed in Table 2 (SEQ ID NOS: 97-100).

### Example 9—Targeted DNA Cleavage by Cas9/sgRNA RNPs Incorporated in LVNPs by C-Terminal Fusion to GagPol

### Aim

To investigate incorporation of SpCas9 in LVNPs.

### Material & Methods

See Example 8

### Results

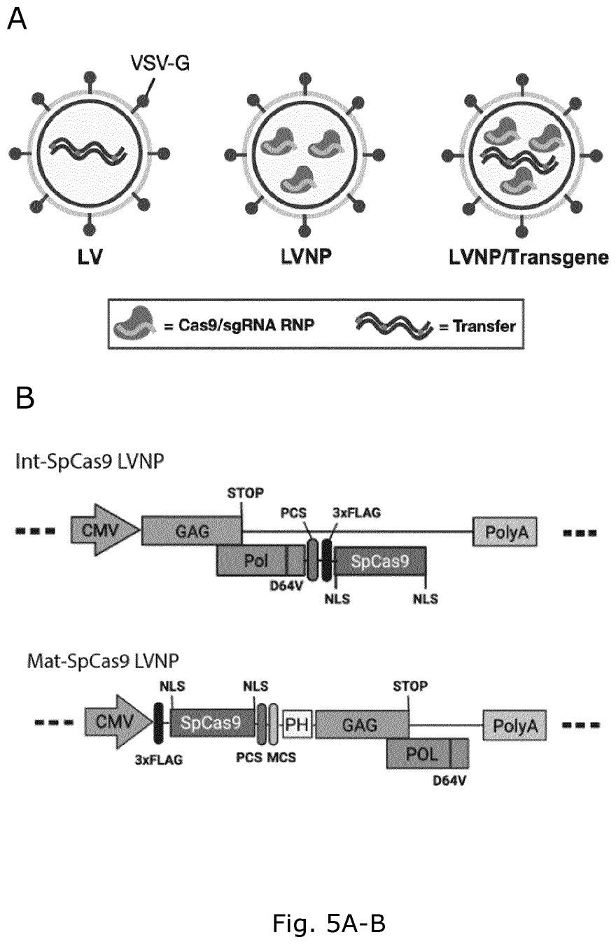

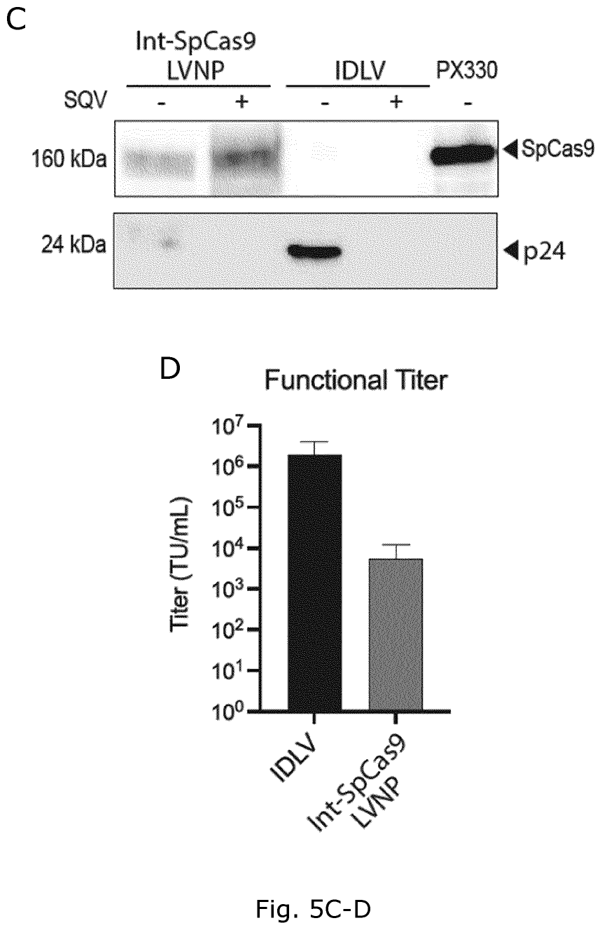

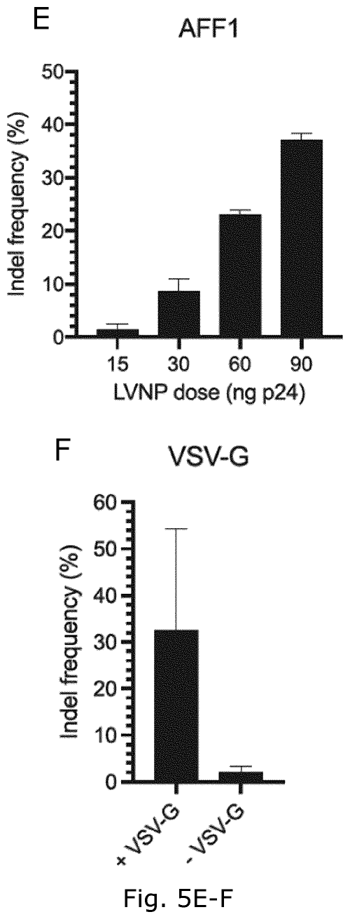

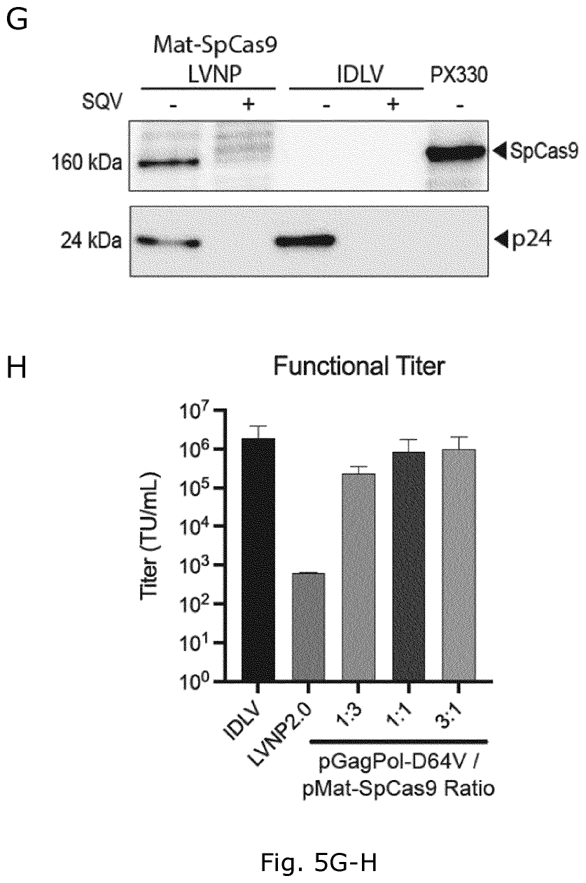

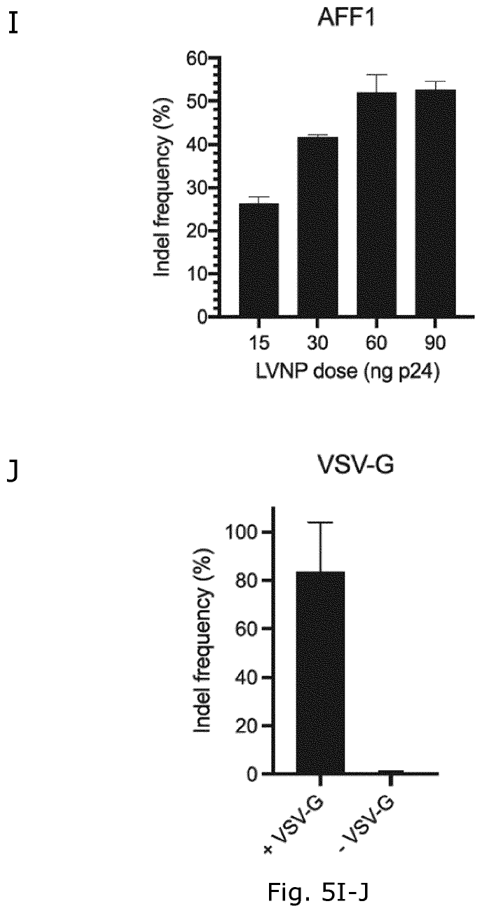

Lentiviruses, including human immunodeficiency virus type 1 (HIV-1), assemble through multimerization of Gag and GagPol polypeptides at the plasma membrane. In conjugation with a dimeric RNA genome, larger aggregates of polypeptides are embedded by a segment of the plasma membrane during budding from virus-producing cells. Released virus particles are immature and undergo maturation triggered by cleavage of the polypeptides by the viral protease. To incorporate Streptococcus pyogenes Cas9 (SpCas9) in LVNPs (FIG. 5A), FLAG-tagged SpCas9 was fused to the integrase protein in the C-terminus of GagPol, based on the rationale that this strategy (referred to as LVNP1.0) was less likely to interfere with virus function due to the restricted number of GagPol molecules, relative to Gag, in the particles.

To facilitate protease-directed release of SpCas9 from GagPol during virion maturation, a protease cleavage site (PCS) was incorporated at the integrase C-terminus (FIG. 5B) and confirmed detection of the 160-kDa SpCas9 protein in LVNP1.0 (FIG. 5C), indicative of effective SpCas9 incorporation and release from GagPol during maturation.

When LVNP1.0 was produced in the presence of saquinavir (SQV), an inhibitor of the protease, release of both p24 and SpCas9 was restricted, confirming that liberation of SpCas9 was directed by HIV-1 protease (FIG. 5C).

Next, utilizing an eGFP-encoding vector, the gene transfer capacity of LVNP1.0 was markedly reduced compared to a standard integrase-defective lentiviral vector (IDLV) without SpCas9 protein (FIG. 5D). To study functional LVNP transfer of SpCas9, two routes for sgRNA delivery was investigated based on (i) packaging of a sgRNA-encoding vector genome or (ii) overexpression of sgRNA in LVNP-producing cells. By packaging a vector containing a U6-driven expression cassette encoding a sgRNA targeting the AFF1 gene (LentiGuide-Puro), modest indel frequencies (˜8%) were observed in HEK293T. In contrast, by co-transfection of a standard sgRNA expression plasmid (U6-sgRNA-CBh-eGFP) together with the remaining plasmids required for LVNP production, disruption of the AFF1 locus was observed in a dose-dependent manner, with a peak indel frequency at ˜38% in HEK293T (FIG. 5E). Notably, indels were not observed following transduction of LVNP1.0 without VSV-G envelope protein (FIG. 5F), suggesting that gene disruption occurred after uptake of LVNP1.0 by VSV-G-directed endocytosis.

### Conclusion

These data demonstrate robust LVNP1.0-directed targeted DNA cleavage following sgRNA overexpression during production

### Example 10—Enhanced Efficacy of SpCas9 Fused to N-Terminus of Gag/GagPol-D64V

### Aim

To investigate the efficacy of SpCas9 fused to the N-terminus of Gag/GagPol-D64V.

### Material & Methods

See Example 8

### Results

FLAG-tagged SpCas9 was fused to the N-terminus of Gag/GagPol harboring an intervening phospholipase C-δ1 pleckstrin homology (PH) domain serving as the membrane anchoring motif (FIG. 5B). Potent incorporation and protease-dependent release of SpCas9 in LVNP2.0 upon maturation was confirmed by Western blotting (FIG. 5G), whereas the gene transfer capacity of this configuration was markedly restricted (FIG. 5H). However, by adding packaging plasmid encoding Gag/GagPol-D64V during LVNP2.0 production, gene transfer could be reconstituted in a dose-dependent manner (FIG. 5H).

Then, SpCas9-loaded LVNP2.0 with the LentiGuide-Puro vector encoding AFF1-targeting sgRNA was produced and higher levels of gene disruption relative to the LVNP1.0 configuration (26% versus 8% indels) was observed. However, in HEK293T cells exposed to LVNP2.0 produced by transient sgRNA overexpression in producer cell, a dose-dependent increase in DNA cleavage in AFF1 leading to >50% indel formation with a dose corresponding to 90 ng p24 was observed (FIG. 5I). In control experiments, VSV-G-dependent uptake of LVNP2.0 resulting in 82% indel formation (90 ng p24) in the AFF1 locus (FIG. 5J) was found.

### Conclusion

These data demonstrate effective co-incorporation of SpCas9 and sgRNA in LVNP2.0, supporting effective delivery and targeted gene disruption in recipient cells.

### Example 11—Modified Scaffolds Enhance LVNP2.0-Directed Gene Disruption

### Aim

To investigate whether LVNP2.0-directed DNA cleavage could be further enhanced by incorporating sgRNAs with improved stability. This could potentially favor the interaction between SpCas9 and sgRNA and reduce sgRNA degradation during LVNP2.0 assembly and maturation.

### Material & Methods

See Example 8

### Results

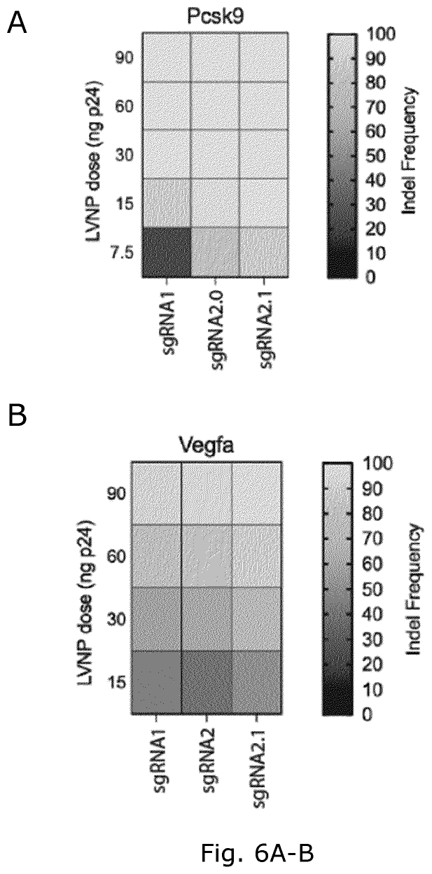

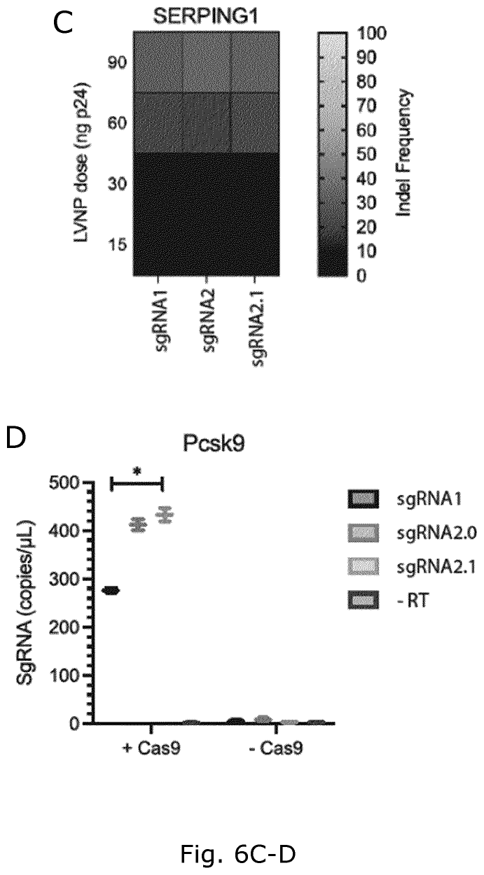

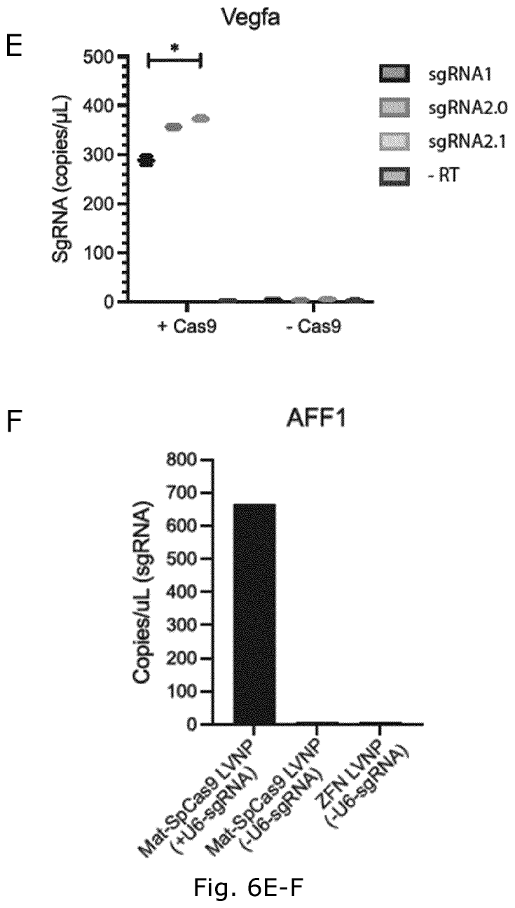

For LVNP2.0, targeting three different genes (Pcsk9, Vegfa (site 1), and SERPING1), two scaffold-optimized sgRNAs (sgRNA2.0 and sgRNA2.1) were compared with the original sgRNA (sgRNA1) (FIG. 3A). For Pcsk9 and Vegfa (site 1), LVNP2.0-directed gene disruption was investigated in murine AML12 hepatocytes and HEK293T cells carrying an inserted Vegfa gene cassette (Holmgaard et al., 2017; Pihlmann et al, 2012), respectively, resulting in complete gene disruption (FIG. 6A-B). In contrast, only 25% indel formation was observed in SERPING1 in HEK293T, indicative of suboptimal sgRNA design (FIG. 6C).

In all cases, indel formation increased with increasing dosages of LVNP2.0. To discriminate performance between sgRNA scaffolds, the dosing was reduced to 7.5 ng to avoid complete disruption of Pcsk9 in murine AML12 hepatocytes (FIG. 6A) and to 60 ng for Vegfa (site 1) in transgenic HEK293T-Vegfa cells. (FIG. 6B). In both cases and in particular at lower doses the sgRNA2.1 scaffold performed best (FIG. 6A-B), whereas no significant differences were observed for SERPING1 (FIG. 6C).

To determine whether the discrepancy in scaffold performance was a result of improved sgRNA incorporation, digital droplet PCR (ddPCR) on RNA isolated from p24-normalized amounts of LVNP2.0 was performed. For LVNP2.0 loaded with sgRNA2.1 for both Pcsk9 and Vegfa (site 1) (FIG. 6D-E), but not SERPING1 (Data not shown), a significant increase in sgRNA abundance was observed.

### Conclusion

These data demonstrate increased sgRNA incorporation and LVNP2.0 efficacy using the sgRNA2.1 scaffold in VSV-G-pseudotyped LVNPs. This scaffold-optimized configuration is also referred to as LVNP2.1.

### Example 12—SpCas9-Dependent sgRNA Incorporation in LVNP2.1

### Aim

To investigate whether incorporation of sgRNA was dependent on co-packaging of SpCas9 protein or a result of random incorporation due to overexpression in the producer cells.

### Material & Methods

See Example 8

### Results

The sgRNA abundance in LVNP2.1 was measured. As a negative control, LVNPs loaded with ZFNs fused to the N-terminus of Gag/GagPol was used. These have previously demonstrated effective protein packaging (Cai Y et al, 2014a).

SpCas9-dependent incorporation of sgRNA in LVNP2.1 was observed, whereas only background levels were observed in ZFN-loaded LVNPs (FIG. 6I). To rule out background signals from plasmid DNA, LVNP2.1 produced by co-transfection with a sgRNA expression plasmid lacking the U6 promoter was included. Only background levels of sgRNA were observed (data not shown), consistent with SpCas9-dependent sgRNA incorporation (FIG. 6I).

### Conclusion

These data demonstrate that pre-assembled RNP complexes are incorporated during assembly of LVNP2.1.

### Example 13—Adjustment LVNP Stoichiometry Enhances LVNP2.1 Performance

### Aim

To maximize LVNP2.1 yield without compromising efficacy.

### Material & Methods

See Example 8

### Results

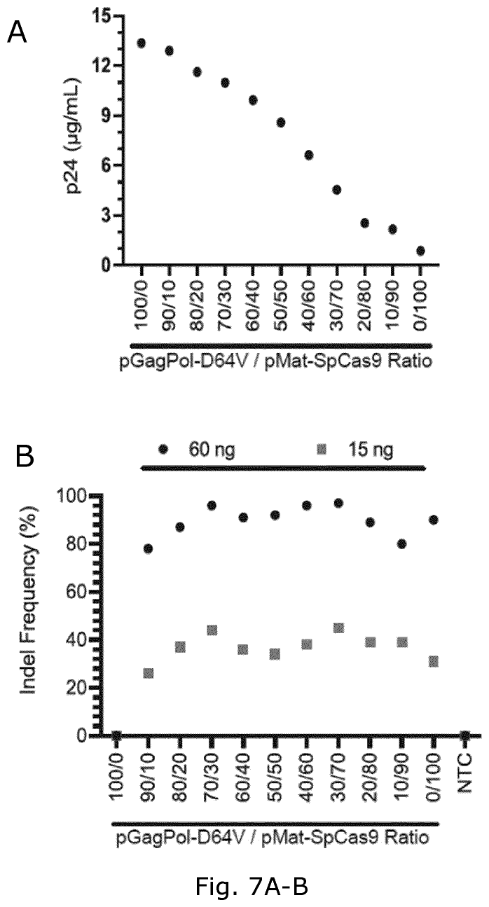

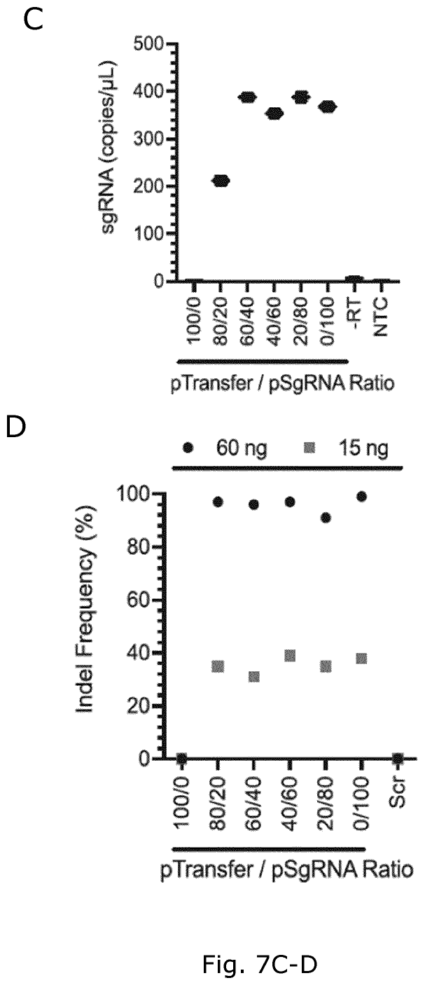

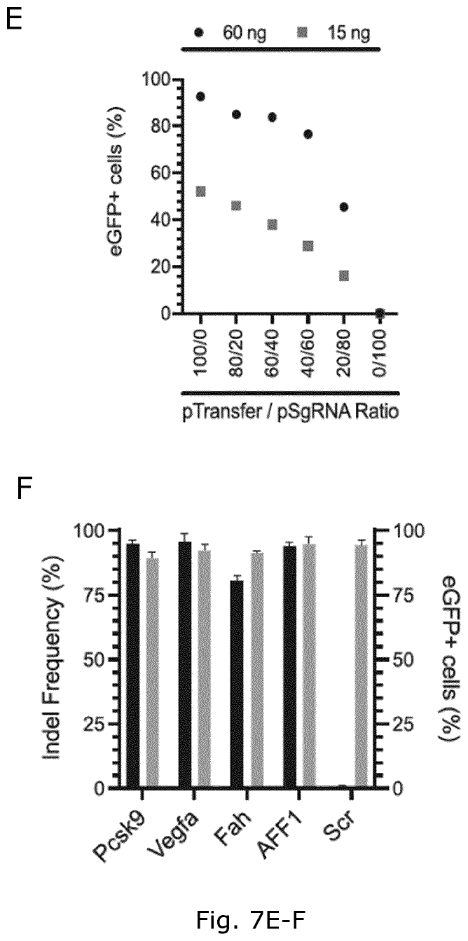

The plasmid stoichiometry during LVNP production was investigated by adjusting the ratio of transfected plasmid DNA. 12 different ratios of the packaging plasmids pLVNP2.0 and pGagPol-D64V during production were tested. Increasing p24 yield was observed with increasing amount of pGagPol-D64V plasmid (FIG. 7A), suggesting that the SpCas9 fusion domain had an overall negative impact on LVNP2.1 production.

However, the indel frequencies in transgenic HEK293T-Vegfa cells (Holmgaard et al., 2017; Pihlmann et al, 2012) ranged from 75 to 98% in cells treated with LVNP2.1 corresponding to 60 ng p24 and from 25-46% with a lower dose (15 ng p24) (FIG. 7B). This suggested that equal amounts of LVNP2.1 (based on p24 measurements) supported comparable levels of DNA cleavage, thus favoring LVNP2.1 compositions with high yield. The 70/30 (pGagPol-D64V/pSpCas9-PH-gagpol-D64V (Mat-SpCas9)) composition was selected as it retains full activity with a negligible drop in yield. To ensure maximum packaging of sgRNA in LVNP2.1, ddPCR was employed to determine the required amount of sgRNA expression plasmid. The sgRNA abundance in LVNP2.1 was largely unaffected (350-400 sgRNA copies/μL) by the ratio between transfer vector plasmid and sgRNA-encoding plasmid, although a significant drop was evident when the sgRNA expression plasmid was reduced to 20% (FIG. 7C). Whereas the indel frequencies were comparable for the six tested configurations (FIG. 7D), a negative correlation between the percentage of eGFP+ cells and the amount of pCCL-PGK-eGFP in the production were found (FIG. 7E).

The 60/40 (pCCL-PGK-eGFP or pCCL-PGK-mCherry/pU6-sgRNA-CBh-eGFP) ratio was chosen to maintain a high rate of gene transfer without compromising RNP formation. To consolidate the optimized configuration (also referred to as LVNP2.2), additional three genes including Fah, Pcsk9, and AFF1 were targeted and a high concordance between the percentage of eGFP+ cells and indel frequency was found (FIG. 7F), providing an option for FACS of transduced cells (Data not shown).

### Conclusion

These data demonstrate that LVNP efficacy can be optimized by modulating stoichiometry of plasmids during production balancing high yields and activity in recipient cells.

### Example 14—LVNP2.2 Supports High On-Target and Low Off-Target DNA Cleavage

### Aim

To investigate the kinetics of LVNP2.2-directed genome editing, a dual-fluorescent reporter system to monitor for endonuclease activity after administration was developed.

### Material & Methods

See Example 8

### Results

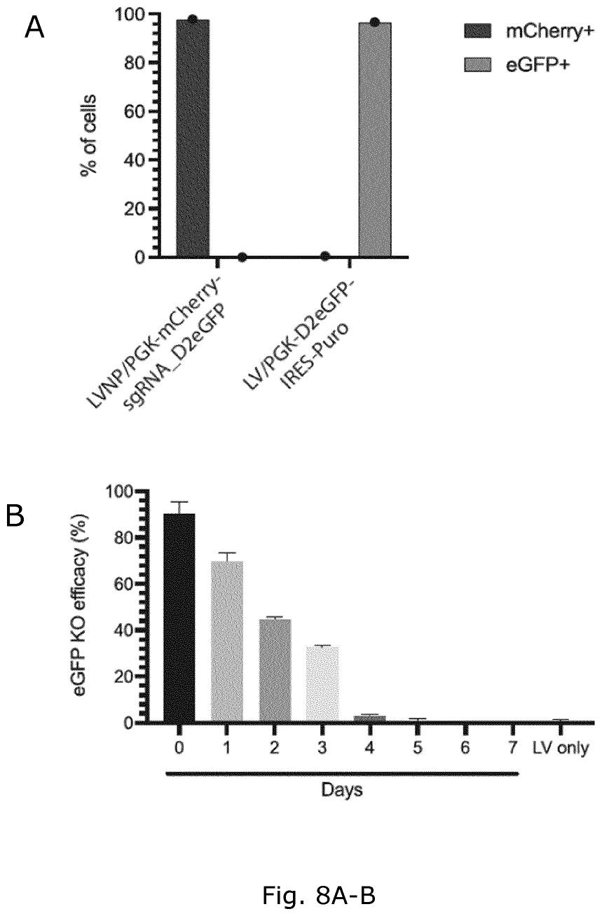

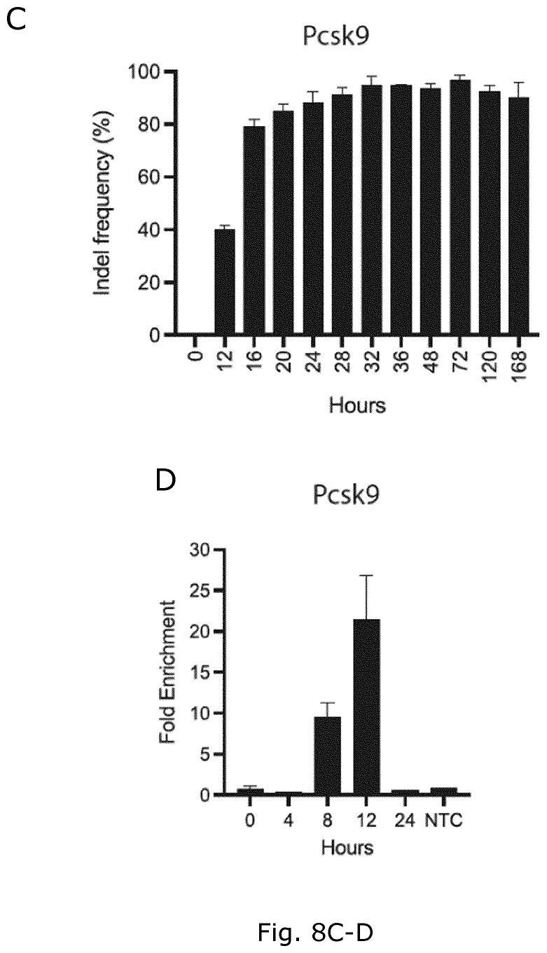

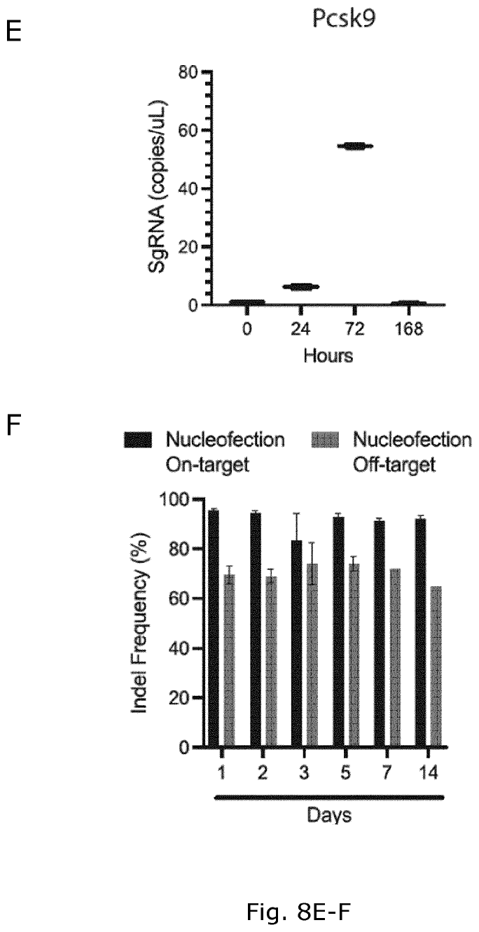

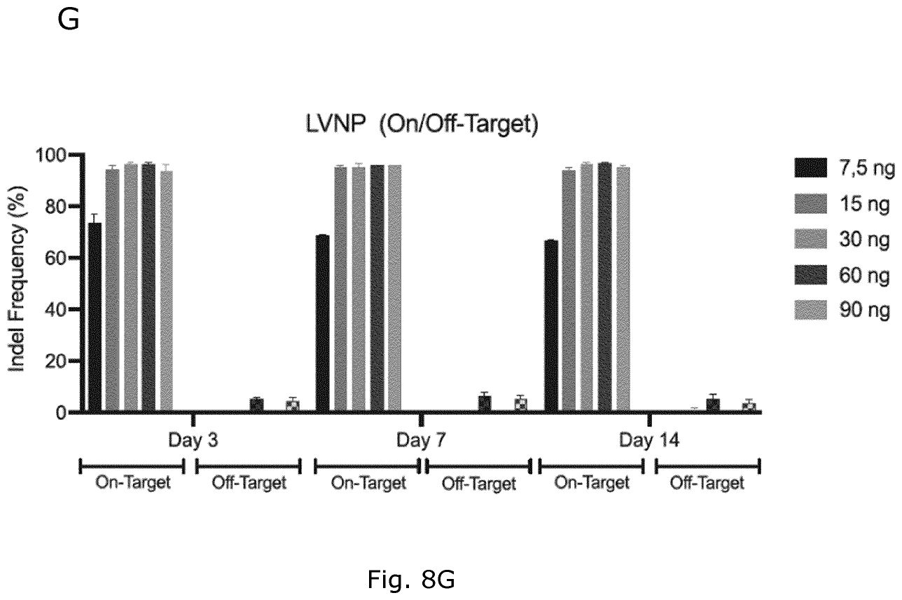

HEK293T were first transduced with LVNP2.2 (loaded with a sgRNA targeting d2eGFP and a transgene vector encoding mCherry) and then at different time points with LV/PGK-d2eGFP-IRES-Puro, allowing the longevity of Cas9/sgRNA RNPs after administration to be evaluated by measuring d2eGFP expression. Using flow cytometry, the emergence of d2eGFP fluorescence after 3 days was analysed (FIG. 8A) and 7 days of puromycin selection (Data not shown). Following co-transduction at day 0, the knockout efficacy reached >95% confirming the high potency of pre-assembled RNP complexes (FIG. 8B). The efficacy decreased in a step-wise and time-dependent manner, with no detectable RNP activity 4 days after LVNP2.2 transduction (FIG. 8B).

The knockout efficacy was calculated as

$$KO Efficacy = 100 \% - \frac{\% D 2 eGFP + cells in LVNP subset ( mCherry + )}{\% D 2 eGFP + cells LV on1y}$$

To determine the ‘time-to-event’ in endogenous loci, the Pcsk9 gene in AML12 cells was targeted and the on/off-target kinetics over the course of 14 days was measured. Consistent with the reporter system, rapid indel formation at the initial readout (12 hours; >40% indels) and >90% indels after 24 hours was observed (FIG. 8C). These results were recapitulated in MRE11 pull down experiments, which demonstrated a ˜10-fold enrichment of double-stranded breaks after 8 hours and >20-fold enrichment after 12 hours (FIG. 8D). In addition, the sgRNA was found to be degraded in recipient cells in a time window ranging from 3 to 7 days (FIG. 8E).

Next, the level of off-target disruption in a well-characterized locus following administration of (i) LVNP2.2 (loaded with a sgRNA targeting Pcsk9 and a transgene vector encoding eGFP) and (ii) nucleofection of recombination SpCas9 complexed with synthetic sgRNA was evaluated. Accompanying molecular analysis revealed >95% on-target and >70% off-target events within 24 hours after SpCas9/sgRNA RNP nucleofection without further accumulation (FIG. 8F).

In contrast, no detectable off-target events in AML12 hepatocytes exposed to 7.5, 15, and 30 ng p24 LVNP2.2 were found, although the level of on-target disruption was comparable to the level observed with RNP nucleofection (FIG. 8G). Off-target cleavage became evident after transduction with LVNP dosages corresponding to 60 and 90 ng p24, but the incidence plateaued at <6% after 72 hours consistent with the decay of LVNP2.2-delivered RNPs (FIG. 8G).

### Conclusion

These data demonstrate very rapid on-target genome editing and suggest that transient and low-abundant RNP delivery by LVNPs induces limited genotoxicity. Using the LVNP2.2 configuration for delivery of a promiscuous sgRNA, we found complete on-target disruption and very limited off-target disruption even at high LVNP doses, whereas state-of-the-art nucleofection resulted in high levels of off-target activity.

### Example 15—Knockout of the Vegfa Gene in the Murine Eye

### Aim

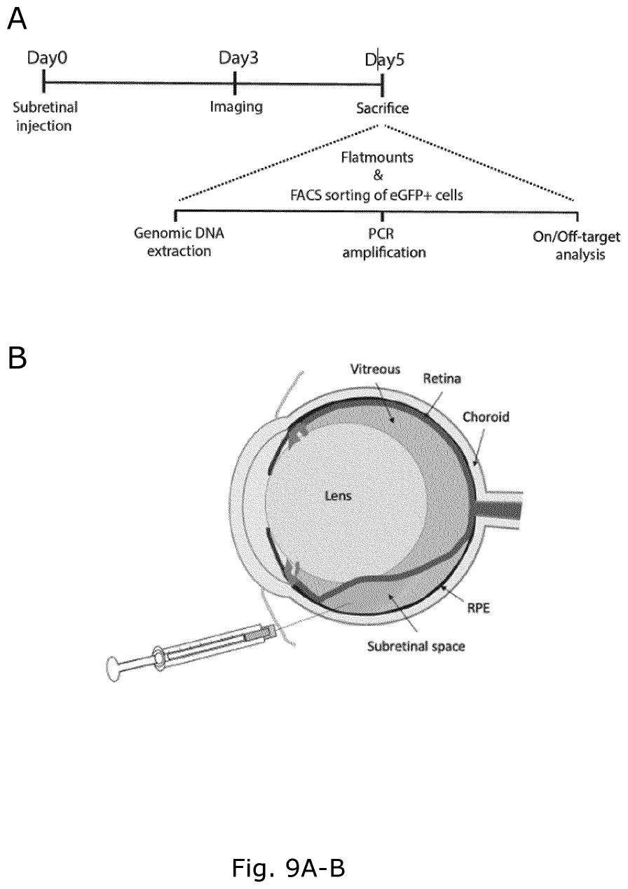

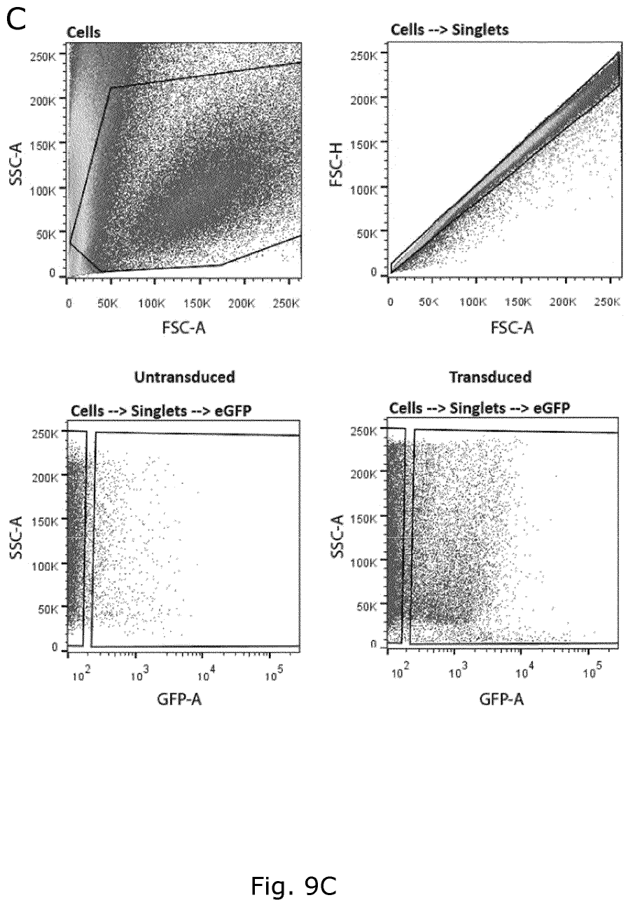

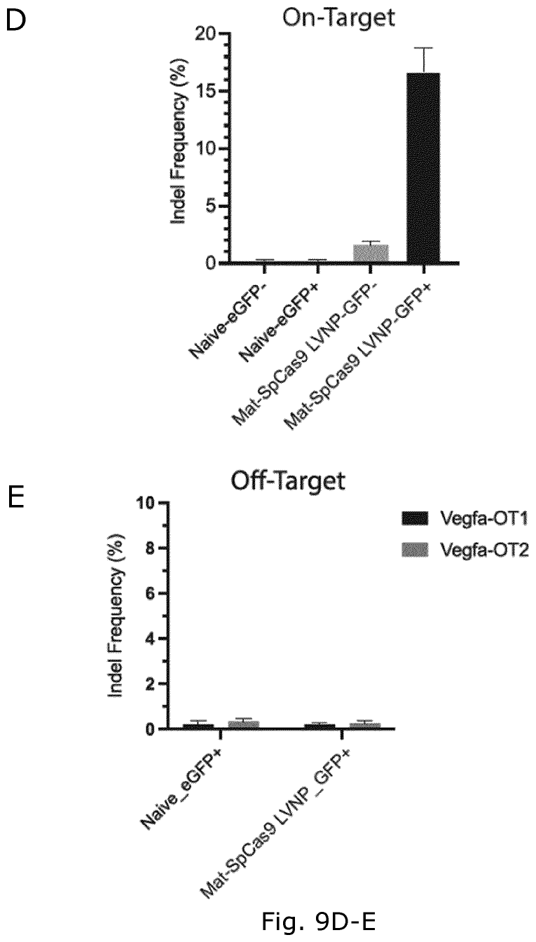

To investigate the effectiveness for in vivo genome editing, Vegfa in retinal pigment epithelial (RPE) cells was targeted in the murine eye (FIG. 9A)

### Material & Methods

See Example 8

### Results

LVNP2.2 loaded with SpCas9, a sgRNA targeting Vegfa (site 1) and vector RNA encoding eGFP) was administered by subretinal injection (n=9) to the left eye (˜16 ng p24) while the right eye served as negative control (FIG. 9B). Each mouse was examined by optical coherence tomography (OCT) and fundoscopy to confirm reattachment of the neuroretina and monitor for eGFP expression (Data not shown). Following sacrifice and enucleation, strong eGFP expression in retinal flatmounts (n=2) consistent with LVNP2.2-directed eGFP gene transfer and high levels of autofluorescence in the red channel indicative of inflammation (Data not shown) was observed.

To evaluate the level of Vegfa knockout, the RPE cells were pooled after retinal dissection of the eye cup (n=6) and separated into eGFP+ or eGFP− populations by FACS (FIG. 9C). Accompanying molecular analysis revealed 17% indel formation in eGFP+ cells (FIG. 9D) without any detectable off-target events in two established off-target loci (FIG. 9E).

Moreover, modest indel frequencies (˜3%) in the eGFP-population was found suggesting that modifications in the Vegfa gene appeared also in cells that did not express eGFP or were omitted due to autofluorescence (Data not shown).

### Conclusion

These data demonstrate that LVNP2.2 is able of in vivo genome editing.

### Abbreviations

CA: Capsid
CRISPR: Clustered Regularly Interspaced Short Palindromic Repeats
eGFP: Enhanced green fluorescent protein
IDLV: Integrase-defective lentiviral vector
IN: Integrase
Int-SpCas9: C-terminal SpCas9 fusion
LHA: Left homology arm
LVNP: Lentivirus-derived nanoparticle
LTR: Long terminal repeat
MA: Matrix
Mat-SpCas9: N-terminal SpCas9 fusion
NC: Nucleocapsid
NHEJ: non-homologous end-joining
PH: phospholipase Cd1 pleckstrin homology domain
PR: Protease
RHA: Right homology arm
RT: Reverse transcriptase
SQV: saquinavir
ZFN: zinc finger nuclease

### REFERENCES

Alsing, S. et al. VEGFA-targeting miR-agshRNAs combine efficacy with specificity and safety for retinal gene therapy. Mol Ther Nucleic Acids 28, 58-76 (2022).
Altschul, S. F. et al. Basic local alignment search tool. J Mol Biol, 215 (3), 403-(1990).
Askou, A. L. et al. Suppression of Choroidal Neovascularization by AAV-Based Dual-Acting Antiangiogenic Gene Therapy. Mol Ther Nucleic Acids 16, 38-50 (2019).
Askou, A. L. et al. Suppression of Choroidal Neovascularization in Mice by Subretinal Delivery of Multigenic Lentiviral Vectors Encoding Anti-Angiogenic MicroRNAs. Hum Gene Ther Methods 28, 222-233 (2017).
Bloh, K. et al. Deconvolution of Complex DNA Repair (DECODR): Establishing a Novel Deconvolution Algorithm for Comprehensive Analysis of CRISPR-Edited Sanger Sequencing Data. Crispr j 4, 120-131 (2021).
Cai, Y. et al. DNA transposition by protein transduction of the piggyBac transposase from lentiviral Gag precursors. Nucleic Acids Res. 42, e28-e28 (2014b).
Cai, Y. et al. Targeted genome editing by lentiviral protein transduction of zinc-finger and TAL-effector nucleases. Elife 3, e01911 (2014a).
Cai, Y. et al. Targeted, homology-driven gene insertion in stem cells by ZFN-loaded ‘all-in-one’ lentiviral vectors. Elife 5, 1-15 (2016).
Choi, J. G. et al. Lentivirus pre-packed with Cas9 protein for safer gene editing. Gene Ther. 23, 627-633 (2016).
Conant, D. et al. Inference of CRISPR Edits from Sanger Trace Data. Crispr j 5, 123-130 (2022).
Cong, L. et al. Multiplex genome engineering using CRISPR/Cas systems. Science 339, 819-23 (2013).
Dull, T. et al. A third-generation lentivirus vector with a conditional packaging system. J Virol, 72, 8463-8471 (1998).
Holmgaard, A. et al. In Vivo Knockout of the Vegfa Gene by Lentiviral Delivery of CRISPR/Cas9 in Mouse Retinal Pigment Epithelium Cells. Mol Ther Nucleic Acids 9, 89-99 (2017).
Holmgaard, A. B. et al. Targeted Knockout of the Vegfa Gene in the Retina by Subretinal Injection of RNP Complexes Containing Cas9 Protein and Modified sgRNAs. Mol Ther 29, 191-207 (2021).
Hsiau, T. et al. (2019) Inference of CRISPR Edits from Sanger Trace Data. bioRXiv (https://doi.org/10.1101/251082)
Jakobsen, M. et al. Amelioration of psoriasis by anti-TNF-alpha RNAi in the xenograft transplantation model. Mol. Ther. 17, 1743-53 (2009).
Kluesner, M. G. et al. EditR: A Method to Quantify Base Editing from Sanger Sequencing. Crispr j 1, 239-250 (2018).
Manceur A. P. et al. Scalable lentiviral vector production using stable HEK293SF producer cell lines. Human Gene Therapy Methods 28 (6), 330-339 (2017).
Neldeborg, S. et al. Rapid and Efficient Gene Deletion by CRISPR/Cas9. Methods Mol. Biol. 1961, 233-247 (2019).
Pihlmann, M. et al. Adeno-associated virus-delivered polycistronic microRNA-clusters for knockdown of vascular endothelial growth factor in vivo. J Gene Med 14, 328-338 (2012).
Ryo et al. Production and validation of lentiviral vectors for CRISPR/Cas9 Delivery. Methods Mol Biol. 1961, 93-109 (2019).
Skipper, K. A. et al. Time-Restricted PiggyBac DNA Transposition by Transposase Protein Delivery Using Lentivirus-Derived Nanoparticles. Mol. Ther. Nucleic Acids 11, 253-262 (2018).
Thomsen, E. A. et al. Identification of BLNK and BTK as mediators of rituximab-induced programmed cell death by CRISPR screens in GCB-subtype diffuse large B-cell lymphoma. Mol. Oncol. 14, 1978-1997 (2020).
Thomasen, E. A. et al. Single-Cell monitoring of activated innate immune signaling by a d2eGFP-based reporter mimicking time-restricted activation of IFNB1 expression. Front Cell Infect Microbiol, 11, 784762 (2022)
Wienert, B., Wyman, S. K., Yeh, C. D., Conklin, B. R. & Corn, J. E. CRISPR off-target detection with DISCOVER-seq. Nat Protoc 15, 1775-1799 (2020).
Xu K. et al. Generation of a stable cell line producing high-titer self-inactivating lentiviral vectors. Mol. Therapy 3 (1), 97-104 (2001).

Sequence Listing

| SEQ ID NO | Name |
|---|---|
| 1 | sgRNA1.0 non-targeting region |
| 2 | sgRNA2.0 non-targeting region |
| 3 | sgRNA2.1 non-targeting region |
| 4 | wt SpCas9 crRNA (FIG. 3A) |
| 5 | wt tracrRNA (FIG. 3A) |
| 6 | sgRNA1 (FIG. 3A) |
| 7 | sgRNA2.0 (FIG. 3A) |
| 8 | sgRNA2.1 (FIG. 3A) |
| 9 | sgRNA1 Aff1 (FIG. 4A) |
| 10 | sgRNA2.0 Aff1 (FIG. 4A) |
| 11 | sgRNA2.1 Aff1 (FIG. 4A) |
| 12 | sgRNA1 pcsk9 |
| 13 | sgRNA2.0 pcsk9 |
| 14 | sgRNA2.1 pcsk9 |
| 15 | sgRNA1 serping1 |
| 16 | sgRNA2.0 serping1 |
| 17 | sgRNA2.1 serping1 |
| 18 | sgRNA1 vegfa (site 3) |
| 19 | sgRNA2.0 vegfa (site 3) |
| 20 | sgRNA2.1 vegfa (site 3) |
| 21 | sgRNA1 eGFP |
| 22 | sgRNA2.0 eGFP |
| 23 | sgRNA2.1 eGFP |
| 24 | MVA9 (Table S1) |
| 25 | MVA10 (Table S1) |
| 26 | MVA64 (Table S1) |
| 27 | MVA65 (Table S1) |
| 28 | MVA66 (Table S1) |
| 29 | MVA67 (Table S1) |
| 30 | MVA111 (Table S1) |
| 31 | MVA113 (Table S1) |
| 32 | MVA114 (Table S1) |
| 33 | MVA115 (Table S1) |
| 34 | MVA141 (Table S1) |
| 35 | MVA142 (Table S1) |
| 36 | AFF1-Sequence (Table S2) |
| 37 | SERPING1-Sequence (Table S2) |
| 38 | eGFPmut-Sequence (Table S2) |
| 39 | VEGFA site 3-Sequence (Table S2) |
| 40 | Pcsk9-Sequence (Table S2) |
| 41 | AFF1-Fw primer (Table S2) |
| 42 | eGFPmut-Fw primer (Table S2) |
| 43 | VEGFA site 3-Fw primer (Table S2) |
| 44 | Pcsk9-Fw primer (Table S2) |
| 45 | AFF1-Rv primer (Table S2) |
| 46 | eGFPmut-Rv primer (Table S2) |
| 47 | VEGFA site 3-Rv primer (Table S2) |
| 48 | Pcsk9-Rv primer (Table S2) |
| 49 | pCCL/PGK-eGFP |
| 50 | pGFP-PH-gagpol-D64V |
| 51 | pLentiGuide-puro |
| 52 | pMDLg/p-RRE-D64V-PCS-SpCas9 |
| 53 | pMDLg/p-PCS-hyPBase |
| 54 | pSpCas9-PH-gagpol-D64V |
| 55 | PZFNL(GFP)-PH-gagpol-D64V |
| 56 | pU6-Chimeric_BB-CBh-eGFP |
| 57 | pU6-SpCas9_sgRNA2.0-CBh-eGFP |
| 58 | pU6-SpCas9_sgRNA2.1-CBh-eGFP |
| 59 | Forward primer (5′-CCTTCAGCTCAGTGACAGTGG-3′) |
| 60 | Reverse primer (5′-CCGACTCGGTGCCACTTT-3′) |
| 61 | Probe (5′-AAATAAGGCTAGTCCGTTATCAACTT-3′) |
| 62 | Extension A (5′-UGCUG-3′) |
| 63 | Extension B (5′-CAGCA-3′) |
| 64 | SERPING1-Fw primer (Table S2) |
| 65 | SERPING1-Rv primer (Table S2) |
| 66 | P1 |
| 67 | P2 |
| 68 | P3 |
| 69 | ddPCR_Pcsk9 Fwd primer (5′-CAGCAGCGGCGGCAACAGGT-3′) |
| 70 | ddPCR_VEGFA Fwd primer (5′-CCTGGAAGATGTCCACCAGT-3′) |
| 71 | ddPCR_Serping1 Fwd primer (5′-TTGCAAGACAGAGGCGAAGT-3′) |
| 72 | ChIP_Pcsk9 Fwd primer (5′-GGCTCCCGTTCTCTCTCTCT-3′) |
| 73 | ChIP_Pcsk9 Rev primer (5′-CTCGGGAAGGACATGGACG-3′) |
| 74 | ChIP Reference Fwd primer (5′-CTGCTCCTGACTGGGTAATGAA-3′) |
| 75 | ChIP Reference Rev primer (5′-ATTTTGTGCTGCATAACCTCCT-3′) |
| 76 | Seq_Vegfa_Nested1_Fwd primer (5′-GAGCCCAGGGAGCAAAGGTCA-3′) |
| 77 | Seq_Vegfa_Nested1_Rev primer (5′-GGTTCCTGGTTGTGCTTAGTGGGA-3′) |
| 78 | Seq_Vegfa_Nested2-P1_Fwd primer |
| | (5′-AAAGGTCACGAAAGCAGATGGTCAA-3′) |
| 79 | Seq_Vegfa_Nested2-P1_Rev primer |
| | (5′-GTGTATATACATAGCTGTCCCCGG-3′) |
| 80 | Seq_Vegfa_Nested2-P2_Fwd primer |
| | (5′-AGAGCTTCGGCAGGGAAGTACA-3′) |
| 81 | Seq_Vegfa_Nested2-P2_Rev primer |
| | (5′-TATTTGATGAGTGGCTGTTGGCCT-3′) |
| 82 | Seq_Vegfa_Nested2-P3_Fwd primer |
| | (5′-TTGTACCACTGTCCTCCTGC-3′) |
| 83 | Seq_Vegfa_Nested2-P3_Rev primer |
| | (5′-GTGGAGCTGTAAGGAGTGGT-3′) |
| 84 | SA1 (Table S1) |
| 85 | SA2 (Table S1) |
| 86 | JH9 (Table S1) |
| 87 | JH10 (Table S1) |
| 88 | JH11 (Table S1) |
| 89 | JH12 (Table S1) |
| 90 | JH13 (Table S1) |
| 91 | JH14 (Table S1) |
| 92 | JH15 (Table S1) |
| 93 | JH16 (Table S1) |
| 94 | D2eGFP-sequence (Table S2) |
| 95 | PCSK9 off target-Fw primer (Table S2) |
| 96 | PCSK9 off target-Rv primer (Table S2) |
| 97 | VEGFA site 1 off target1-Fw primer (Table S2) |
| 98 | VEGFA site 1 off target1-Rv primer (Table S2) |
| 99 | VEGFA site 1 off target2-Fw primer (Table S2) |
| 100 | VEGFA site 1 off target2-Rv primer (Table S2) |
| 101 | Fah-sequence (Table S2) |
| 102 | Fah-Fw primer (Table S2) |
| 103 | Fah-Rv primer (Table S2) |
| 104 | sgRNA2.1 d2eGFP |
| 105 | sgRNA2.1 Fah |
| 106 | pCCL/PGK-MCS-IRES-puro |
| 107 | pT2/UASTK-d2eGFP-SV40-neo |
| 108 | pCCL-PGK-mCherry |
| 109 | pCCL/PGK-FahMut-P2A-eGFP-IRES-puro |
| 110 | VEGFA site 1-sequence (Table S2) |
| 111 | VEGFA site 1-Fw primer (Table S2) |
| 112 | VEGFA site 1-Rv primer (Table S2) |
| 113 | sgRNA2.1 VEGFA(site 1) |

TABLE S1
Sequences of oligonucleotides used for construction of plasmids

| Name | Sequence |
|---|---|
| MVA9 | GAGTGCTGGCCGAGGCCATGAGCgactataaggaccacgacg |
| MVA10 | CCTGCGGCCGCTCCGGAATTCCATGTGTCACTTTTTCTTTTTTGCCTG |
| MVA64 | GATCCGCTAGCGCTACCGGTACCACCATGGACTATAAGGAC |
| MVA65 | CTCGAGATCTGAGTCCGGTCTGCACAATCGGATAGTTCTGGCTAAAGTTCTTTTTCTTTTTTGCCTGGCCG |
| MVA66 | TCACTTTTTTTCAGGTTGGACCGGTCGCCACCATGGTG |
| MVA67 | GCTGATCAGCGAGCTCTAGGCTTGTACAGCTCGTCCATGCC |
| MVA111 | AATTATGTTTTAAAATGGACTATCATATGCTTACCGTAACTTGAAAGTATTTCG |
| MVA113 | GGGGCGTACTTGGCATATGATACACTTGATGT |
| MVA114 | CTTATTTAAACTTGCTATGCTGTTTCCAGCATAGCTCTTAAACAGGTCT |
| MVA115 | GGAAACAGCATAGCAAGTTTAAATAAGGCTAGTCCGTTA |
| MVA141 | CCACTTTGCTGTTTCCAGCAAAGTTGATAACGGACTAGCCTTATTTAAA |
| MVA142 | AACTTTGCTGGAAACAGCAAAGTGGCACCGAGTCGG |
| SA1 | TGTCGACGATATCTTCGAACATGGTGAGCAAGGGCGAG |
| SA2 | ACGGCCGCTATGCTTTACTGCTACACATTGATCCTAGCAGAAG |
| JH9 | CTCTTCCTCATCTCCGGGCCTTTCGACCTCTAGCGAATGTCCTTTATTCCAGTGGCCG |
| JH10 | GCTGGGCCACCAGGCATTACTGCTCCAGTCGTTCATGAGGAC |
| JH11 | CCTCATGAACGACTGGAGCAGTAATGCCTGGTGGCCCAG |
| JH12 | CCATTGCTGGATGTCTCGTGCTATGAAAAGACAGCAAGCTTAGGTCT |
| JH13 | AGCTTGCTGTCTTTTCATAGCACGAGACATCCAGCAATGGG |
| JH14 | GAGAAGTTTGTTGCGCCGGATCCGGCTGGTGAAAGGGCAGGCAG |
| JH15 | TGCCTGCCCTTTCACCAGCCGGATCCGGCGCAACAAACT |
| JH16 | GCGGAATTTACGGCCGCTATGCTTTACTGCCTTGTACAGCTCGTCCATG |

TABLE S2
Sequences of sgRNAs used in the study and sequencing primers

| | Sequence | | Rv primer |
|---|---|---|---|
| Target | (Spacer sequence) | Fw primer | |
| AFF1 | CCTTCAGCTCAGTGACAGTG | AGAGGCTTGCACTTAACTGGT | AAACACTGGGCTCGTCAGTT |
| SERPING1 | GTTTGCAAGACAGAGGCGAA | CTCATCTGCCGCACTGTCAGAAAT | GATTGGTGACTCTTATGGGAGTGTC |
| | | TACTCT | CAACA |
| eGFPmut | CTGCTTCATGTTAGGGGTAG | ATTGACGCAAATGGGCGGTAG | TTACTTGTACAGCTCGTCCATG |
| | | | CCG |
| VEGFA site 3 | GGTGAGTGAGTGTGTGCGTG | CTCAGCTCCACAAACTTGGTGCC | AGCCCGCCGCAATGAAGG |
| Pcsk9 | AGCAGCAGCGGCGGCAACAG | TCTCTCTTTCTGAGGCTAGAGGA | CCCCAAGTCTATTTCCAAGCCG |
| d2eGFP | CGGTGAACAGCTCCTCGCCC | NA | NA |
| PCSK9 Off target | NA | GTACAAGGCTCCACAGGTCA | CTCCTGCTCTAGGAGAGGTGT |
| VEGFA site 1 | CTCCTGGAAGATGTCCACCA | GAGCCCAGGGAGCAAAGGTCA | GGTTCCTGGTTGTGCTTAGTGGGA |
| VEGFA site 1 Off | NA | GGAGACTGTTTGGCCTCTGT | AATAGTCCCCTGTTCGTGCC |
| target1 | | | |
| VEGFA site 1 Off | NA | GCTGGTCCCTGTGGATTACA | TGAGAGACTTCGGGGACAGA |
| target2 | | | |
| Fah | GGATGGTCCTCATGAACGAC | GTTGCCAAATTGGCTCCACTTAC | CCACACCCACAGAGTCAGAA |

1. A lentivirus-derived particle comprising one or more Cas9-like proteins and at least one optimized sgRNA, wherein the optimized sgRNA comprises a targeting region and a non-targeting region, wherein said non-targeting region comprises a nucleotide sequence corresponding to SEQ ID NO: 1 or sequences having at least 90% sequence identity to SEQ ID NO: 1, said nucleotide sequence further comprising at least the following modifications
an extended repeat-anti-repeat region comprising a first extension of 2-8 base pairs in the repeat-anti-repeat region corresponding to nucleotides 1-12 and 17-30 of SEQ ID NO: 1;
optionally, an extended stem-loop 2 region comprising a second extension of 2-8 base pairs in the stem-loop 2 corresponding to the nucleotides 48-61 of SEQ ID NO: 1; and/or
optionally, an A-U flip of the nucleotides corresponding to nucleotides 5 and 36 of SEQ ID NO: 1.

2. The particle according to claim 1, wherein said extended repeat-anti-repeat region comprises a first extension of 3-7 base pairs.

3. The particle according to claim 1, wherein said first extension is inserted 3′ to nucleotide 12 and 5′ to nucleotide 17 of SEQ ID NO: 1.

4. The particle according to claim 1, wherein said first extension consists of the following nucleotides 2-8 5′-UGCUG-3′ (SEQ ID NO: 62) inserted 3′ to nucleotide 12 of SEQ ID NO: 1 and 5′-CAGCA-3′ (SEQ ID NO: 63) inserted 5′ to nucleotide 17 of SEQ ID NO: 1.

5. The particle according to claim 1, comprising an extended stem-loop 2 region comprising a second extension of base pairs in the stem-loop 2 corresponding to the nucleotides 48-61 of SEQ ID NO: 1; and wherein said second extension is inserted 3′ to nucleotide 52 and 5′ to nucleotide 56 of SEQ ID NO: 1.

6. The particle according to claim 1, comprising an extended stem-loop 2 region comprising a second extension of 2-8 base pairs in the stem-loop 2 corresponding to the nucleotides 48-61 of SEQ ID NO: 1.

7. The particle according to claim 1, comprising an extended stem-loop 2 region comprising a second extension of 2-8 base pairs in the stem-loop 2 corresponding to the nucleotides 48-61 of SEQ ID NO: 1; and
wherein said second extension is inserted 3′ to nucleotide 52 and 5′ to nucleotide 56 of SEQ ID NO: 1; and wherein said second extension consists of the following nucleotides 5′-UGCUG-3′ (SEQ ID NO: 62) inserted 3′ to nucleotide 52 of SEQ ID NO: 1 and 5′-CAGCA-3′ (SEQ ID NO: 63) inserted 5′ to nucleotide 56 of SEQ ID NO: 1.

8. The particle according to claim 1, wherein said non-targeting region comprises a nucleotide sequence of SEQ ID NO: 2 or 3 or sequences having at least 90% sequence identity to SEQ ID NO: 2 or 3.

9. The particle according to claim 1, wherein said Cas9-like protein is fused to the N-terminal of the GagPol polypeptide (Mat-Cas9).

10. The particle according to claim 1, comprising an A-U flip of the nucleotides corresponding to nucleotides 5 and 36 of SEQ ID NO: 1.

11. A method of producing a lentivirus-derived particle according to claim 1, wherein said method comprises the steps of
a) providing packaging plasmid(s) including a packaging plasmid comprising at least gagpol viral genes under the control of a promoter;
b) providing a nucleic acid capable of transcribing at least one sgRNA;
c) transfecting said packaging plasmid(s) and said nucleic acid constructs into a producer cell;
d) purifying lentivirus-derived particles from said producer cell.

12. The method according to claim 11, wherein at least two packaging plasmids being a first plasmid comprising gagpol viral genes under the control of a promoter and a second plasmid comprising a nucleic acid encoding for a Cas9-like protein, are provided.

13. The method according to claim 1, wherein at least two packaging plasmids being a first plasmid comprising gagpol viral genes under the control of a promoter and second plasmid comprising a nucleic acid encoding for a Cas9-like protein, are provided; and
wherein the ratio between said first plasmid and said second plasmid is 10:90 to 90:10.

14. A method of amending the genome of a cell by contacting a cell to be amended with a lentivirus-derived particle according to claim 1.

15. The method according to claim 14, being performed in vitro.

16. The method according to claim 14, wherein said lentivirus-derived particle has an indel formation of at least 50%.

17. The method according to claim 14, for the prevention, alleviation and/or treatment of diseases of the eye.

18. The method according to claim 14, for the prevention, alleviation and/or treatment of diseases of the eye selected from the group consisting of age-related macular degeneration, Leber's congenital amaurosis, and retinitis pigmentosa.
